(19) United States
(12) Patent Application Publication
(10) Pub. No.: US 2016/0074390 A1
(43) Pub. Date: Mar. 17, 2016

(54) HUMAN DOSING OF PHOSPHATASE INHIBITOR

(71) Applicant: JOHN S. KOVACH, EAST SETAUKET, NY (US)

(72) Inventor: JOHN S. KOVACH, EAST SETAUKET, NY (US)

(73) Assignee: Lixte Biotechnology, Inc., East Setauket, NY (US)

(21) Appl. No.: 14/852,219

(22) Filed: Sep. 11, 2015

Related U.S. Application Data
(60) Provisional application No. 62/209,078, filed on Aug. 24, 2015.
(60) Provisional application No. 62/118,265, filed on Feb. 19, 2015.
(60) Provisional application No. 62/049,608, filed on Sep. 12, 2014.

(51) Int. Cl. A61K 31/496; A61K 33/24; A61K 31/475; A61K 31/337; A61K 31/4188; A61K 31/44; A61K 45/06; A61K 31/704

(52) U.S. Cl. CPC A61K 31/496; A61K 45/06; A61K 33/24; A61K 31/704; A61K 31/337; A61K 31/4188; A61K 31/44; A61K 31/475

(57) ABSTRACT

The present invention provides a method of inhibiting protein phosphatase 2A (PP2A) in a human subject in need thereof comprising administering to the subject an amount of from 0.1 mg/m² to 5 mg/m² of a compound having the structure

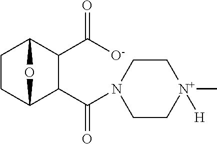 [SMILES: C[NH+]1CCN(C(=O)C2C(C(=O)[O-])[C@H]3CC[C@@H]2O3)CC1]

or a salt, zwitterion, or ester thereof, so as to thereby inhibit protein phosphatase 2A (PP2A) in the subject.

This application claims the benefit of U.S. Provisional Application Nos. 62/209,078, filed Aug. 24, 2015; 62/118,265, filed Feb. 19, 2015; and 62/049,608, filed Sep. 12, 2014, the contents of each of which are hereby incorporated by reference.

Throughout this application various publications are referenced. The disclosures of these documents in their entireties are hereby incorporated by reference into this application in order to more fully describe the state of the art to which this invention pertains.

### BACKGROUND OF THE INVENTION

Protein phosphatase 2A (PP2A), a family of the major serine/threonine phosphatases in cells, is widely considered a tumor suppressor (Van Hoof, C. et al. 2004; Westermarck, J. et al. 2008). Inhibition of PP2A is thought to be a precursor of malignant transformation of human cells and some PP2A inhibitors such as okadaic acid are associated with tumorigenesis and tumor progression (Junttila, M. R. et al. 2007; Suganuma, N. et al. 1988). Structurally, PP2A has three subunits and each subunit has alternative isoforms (Mumby, M. 2007), resulting in over 60 heterotrimeric holoenzymes (Gwinn, D. et al. 2013). Because of the complicated constitutive and various signaling pathways involving PP2A, this ubiquitous phosphatase may play distinct roles in different tissue and disease states. For instance, the B55α regulatory subunit of PP2A was shown to enhance the survival of human fibrosarcoma cells during glutamine deprivation (Reid, M. A. et al. 2013), while inhibition of the B56γ subunit induces tumorigenic transformation of human embryonic kidney cells (Chen, W. et al. 2004), thereby acting like 856a as a tumor suppressor (Arnold, H. K. et al. 2008). This diversity of PP2A function in tumorigenesis suggests in certain circumstances targeting PP2A may be an effective cancer strategy.

Cantharidin, a natural product isolated from Mylabris sidae, and several cantharidin derivatives have PP2A inhibitory activity, and have been used as anti-cancer agents for decades (Hart, N. E. et al. 2004; Li, W. et al. 2010; Liu, D. et al. 2009; McCluskey, A. et al. 2000). The mechanism by which PP2A exerts anti-cancer activity is believed be abrogation of cell cycle checkpoints and induction of mitotic catastrophe (Kalev, P. et al. 2011). Pharmacologic inhibition of PP2A has previously been shown to sensitize cancer cells to radiation-mediated DNA damage via constitutive phosphorylation of various signaling proteins, such as p53, γH2AX, PLK1 and Akt, resulting in cell cycle deregulation, inhibition of DNA repair, and apoptosis (Wei, D. et al. 2013). Although cantharidin has previously been used in the treatment of hepatomas and has shown efficacy against multidrug-resistant leukemia cell lines (Efferth, T. et al. 2002), its severe toxicity limits its clinical usefulness. LB100 is a small molecule derivative of cantharidin with significantly less toxicity. Previous pre-clinical studies have shown that LB100 can enhance the cytotoxic effects of temozolomide, doxorubicin, and radiation therapy against glioblastoma (GBM), metastatic pheochromocytoma, and pancreatic cancer (Wei, D. et al. 2013; Lu, J. et al. 2009; Zhang, C. et al. 2010; Martiniova, L. et al. 2011). LB100 is also undergoing a phase 1 study in combination with docetaxel for the treatment of solid tumors (Chung, V. 2013).

### Mad2 Overexpressing Cancers

Mitotic arrest deficiency 2 (Mad2), an essential component of the spindle checkpoint, is overexpressed in many cancers, such as hepatocellular carcinoma (Zhang, S. H. et al. 2008), human osteosarcoma (Yu, L. et al. 2010), primary liver cancer (Yi, C. et al. 2000), gastric cancer (Tanaka, K. et al. 2001; Wang, L. et al. 2009), ovarian cancer (Nakano, Y. et al. 2012), endometrial cancer (Li, L. et al. 2013), colorectal cancer (Wassman, K. et al. 2003), lung cancer (Kato, T. et al. 2011), soft-tissue sarcoma (Hisaoka, N. et al. 2008), seminomas (Fung, N. K. et al. 2007), and breast cancer (Agoston, A. T. et al. 2007). The spindle checkpoint is a surveillance mechanism that ensures faithful chromosome segregation during mitosis. During the process of cell division, the spindle checkpoint prevents separation of duplicated chromosomes until the chromosome are properly attached to the spindle apparatus. When the spindle checkpoint malfunctions, it can lead to chromosome missegregation, aneuploidy and tumorigenesis (Musacchio, A. et al. 2007).

Mice genetically engineered to overexpress Mad2 developed chromosome instability (CIN) and aneuploidy (Sotillo et al. 2007). High levels of Mad2 also caused the formation of aggressive tumors in multiple organs (Schartzman, J. M. et al. 2010). Other studies show that overexpression of Mad2 is caused by loss of the tumor suppressor Rb or p53 (Schartzman, J. M. et al. 2011; van Deursen, J. M. 2007). It has also been proposed that inhibition of PP2A increases Mad2 phosphorylation and inhibits the growth of certain Mad2-overexpressing cancer cells (Bian et al. 2014).

Additional cancers that overexpress Mad2 include mucinous ovarian cancer (Nakano et al. 2012), urothelial bladder cancer (Choi et al. 2013), squamous cell carcinoma of the uterine cervix (Kim et al. 2014), diffuse large cell lymphoma and others (Sotillo et al. 2007).

### Triple Negative Breast Cancer

Triple-negative breast cancers (TNBCs) comprise 15% to 20% of breast cancers in Western countries. TNBC is defined as the absence of staining for estrogen receptor, progesterone receptor, and HER2/neu (Silver, D. P. et al. 2010). TNBC is insensitive to some of the most effective therapies available for breast cancer treatment including HER2-directed therapy such as trastuzumab and endocrine therapies such as tamoxifen or the aromatase inhibitors. Combination cytotoxic chemotherapy administered in a dose-dense or metronomic schedule remains the standard therapy for early-stage TNBC (Mehta, R. S. et al. 2008). Approximately 70% of breast cancers in individuals carrying a germline BRCA1 mutation are triple negative.

### Diseases Characterized by Loss of Protein Function

Various genetic disorders are caused by mutations in DNA that produce proteins with abnormal amino acid sequences, which cause the proteins to misfold. Such misfolding, often caused by genetic mutations, does not allow the protein to assume its correct functional shape or conformation. In disorders that result in loss-of-function, the misfolded proteins are unable to function properly and targeted for early destruction. This scenario causes a disruption of proteostasis. In gain-of-function diseases, the misfolded proteins are not destroyed. Instead, the misfolded proteins are broken down by the cell and reassembled into aggregates which cause cellular damage. Gain-of-function diseases, e.g. Gaucher's disease and Von Hippel-Lindau Disease, are often associated with aging but are also caused by genetic mutations.

### Type-2 Diabetes

Diabetes mellitus (diabetes) is a complex chronic disease characterized by high levels of blood glucose resulting from defects in insulin secretion and/or insulin action. In order to function properly, the human body must have a balanced production of insulin from the pancreas to transport glucose efficiently to other organs and tissues for storage. Any insulin imbalance or loss of sensitivity can cause a chronic overabundance of glucose leading to diabetes.

Diabetes is associated with various, and often serious complications that may lead premature death. Diabetics are more likely to suffer from heart disease, kidney disease, eye disease including blindness, peripheral vascular disease at times requiring amputation of the leg, stroke, and are more likely to die of complications of flu and pneumonia than non-diabetics. Other conditions related to diabetes include nervous system diseases, which often includes impaired sensation or pain in the feet or hands, slowed digestion of food in the stomach, carpal tunnel syndrome, periodontal disease, and complications of pregnancy, diabetic ketoacidosis and hyperosmolar nonketotic coma.

Type 2 diabetes, in particular, is one of the major medical problems facing populations throughout the world. In the United States, approximately 15% of the adult population is believed to have type 2 diabetes. This incidence is steadily increasing. It has recently been reported that even children are now being diagnosed with type 2 diabetes, a phenomenon that has almost been unheard of in the past. In type 2 diabetes, the ability of insulin to decrease blood glucose levels is impaired and overcoming this insulin resistance is a major goal in type 2 diabetes.

### Reperfusion Injury

Reperfusion is a re-establishment of blood flow and re-oxygenation of an affected area following an ischemic event and is critical to limit irreversible damage. However, the absence of oxygen and nutrients from the blood creates a condition in which reperfusion injury may occur. The restoration of blood flow after an ischemic event results in inflammation and oxidative damage. Upon restoration of blood flow, white blood cells release inflammatory factors such as interleukins as well as free radicals. The restored blood flow reintroduces oxygen within cells that damages cellular proteins, DNA, and the plasma membrane.

As acute myocardial infarction (MI) remains the leading cause of death worldwide, the possibility that a pharmacologic intervention applied promptly but after the onset of the heart attack would minimize damage to heart tissue caused by reperfusion and therefore be expected to save many lives and reduce the number of individuals with heart failure following excessive cardiac muscle damage after an MI (Yellon and Hausenloy, 2005; Longacre et al, 2011). It has been suggested that a lack of commercial interest in a developing a drug that would likely be used only once in an individual has limited progress in this field (Cohen and Downey, 2011).

At present, the only established intervention that consistently reduces the size of myocardial infarcts in humans is by improving coronary artery flow as soon as possible after an MI either by drugs, which dissolve fresh clots and/or cardiac catheterization with balloon angioplasty with or without placement of a fixed conduit, a stent. These methods of improving coronary artery blood flow (reperfusion) have improved patient care and decreased hospital mortality. However, delay in initiating reperfusion because of travel time to a cardiac center is a serious limitation to applying these treatments to patients with acute cardiac injury. It has also been discovered the reperfusion treatment in and of itself may cause myocardial cell death, a phenomenon called reperfusion injury. Reducing injury caused by reperfusion by pharmacologic means should improve the success of current interventions for acute heart attacks. A drug minimizing tissue damage that could be administered at the time of a MI by emergency personnel prior to arrival at a cardiac center could be a major advance in the care of heart attack victims. Acute injury due to oxygen deprivation leading to myocardial damage is also a significant problem in heart surgery. The incidence of infarction after coronary artery bypass graft surgery has been estimated to be as high as 191 with attendant cardiac morbidity (Longacre et al, 2011).

### SUMMARY OF THE INVENTION

The present invention provides a method of inhibiting protein phosphatase 2A (PP2A) in a human subject in need thereof comprising administering to the subject an amount of from 0.1 mg/m² to 5 mg/m² of a compound having the structure

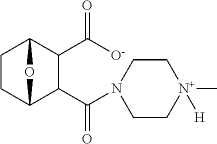 [SMILES: C[NH+]1CCN(C(=O)C2C(C(=O)[O-])[C@H]3CC[C@@H]2O3)CC1]

or a salt, zwitterion, or ester thereof, so as to thereby inhibit protein phosphatase 2A (PP2A) in the subject.

### BRIEF DESCRIPTION OF THE FIGURES

FIG. 1A: MAD2 immunohistochemical stains of the pancreatic cancer (of Patient 10) at 100× magnification. Formalin Fixed Paraffin Embedded (FFPE) tissue was sectioned at 5 microns. Sections were dewaxed and after treatment with Epitope Retrieval 2 (EDTA; Leica, Buffalo, Ill.), stained with antibody to MAD2L1.

FIG. 1B: MAD2 immunohistochemical stains of the pancreatic cancer cancer (of Patient 10) at 400× magnification. Formalin Fixed Paraffin Embedded (FFPE) tissue was sectioned at 5 microns. Sections were dewaxed and after treatment with Epitope Retrieval 2 (EDTA; Leica, Buffalo, Ill.), stained with antibody to MAD2L1.

### DETAILED DESCRIPTION OF THE INVENTION

The present invention provides a method of inhibiting protein phosphatase 2A (PP2A) in a human subject in need thereof comprising administering to the subject an amount of from 0.1 mg/m² to 5 mg/m² of a compound having the structure

or a salt, zwitterion, or ester thereof, so as to thereby inhibit protein phosphatase 2A (PP2A) in the subject.

In one embodiment of the above method, the compound

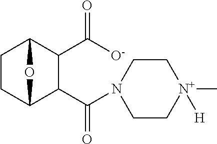 [SMILES: C[NH+]1CCN(C(=O)C2C(C(=O)[O-])[C@H]3CC[C@@H]2O3)CC1]

is replaced with any of the compounds disclosed in the present application.

In one embodiment, the subject in need thereof is afflicted with a disease or condition mediated by normal expression, overexpression, or under expression of protein phosphatase 2A (PP2A).

In another embodiment, the amount of the compound treats the disease or condition mediated by the normal expression, overexpression, or under expression of protein phosphatase 2A (PP2A).

In one other embodiment, the inhibition of protein phosphatase 2A (PP2A) in the subject treats the disease or condition mediated by the normal expression, overexpression, or under expression of protein phosphatase 2A (PP2A).

In yet another embodiment, the disease or condition mediated by the normal expression, overexpression, or under expression of protein phosphatase 2A (PP2A) is cancer, a reperfusion injury, a disease characterized by loss of protein function or type-2 diabetes.

In some embodiments, the disease or condition mediated by the normal expression or under expression of protein phosphatase 2A (PP2A) is a cancer.

In some embodiments, the disease or condition mediated by overexpression of protein phosphatase 2A (PP2A) is a reperfusion injury, a disease characterized by loss of protein function or type-2 diabetes.

The present invention also provides a method of treating cancer, reperfusion injury, a disease characterized by loss of protein function, a neurodegenerative disease or type-2 diabetes in a human subject comprising administering to the subject an amount of from 0.1 mg/m² to 5 mg/m² of a compound having the structure

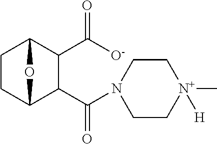 [SMILES: C[NH+]1CCN(C(=O)C2C(C(=O)[O-])[C@H]3CC[C@@H]2O3)CC1]

or a salt, zwitterion, or ester thereof, so as to thereby treat the cancer, reperfusion injury, a disease characterized by loss of protein function, a neurodegenerative disease or type-2 diabetes.

In some embodiments, the amount of the compound administered is 0.1 mg/m² to 5 mg/m².

In some embodiments, the amount of the compound administered is 0.25 mg/m² to 2.5 mg/m².

In some embodiments, the amount of the compound administered is 2.5 mg/m² to 5 mg/m².

In some embodiments, the amount of the compound administered is 3 mg/m² to 4.5 mg/m²

In some embodiments, the amount of the compound administered is 0.25 mg/m², 0.5 mg/m², 0.83 mg/m², 1.25 mg/m², 1.75 mg/m² or 2.33 mg/m².

In some embodiments, the amount of the compound administered is about 0.25 mg/m², 0.5 mg/m², 0.75 mg/m², 1.0 mg/m², 1.25 mg/m², 1.5 mg/m², 1.75 mg/m², 2.0 mg/m², 2.25 mg/m², 2.5 mg/m² or 2.75 mg/m².

In some embodiments, the amount of the compound administered is about 3 mg/m², 3.25 mg/m², 3.5 mg/m², 3.75 mg/m², 4 mg/m², 4.25 mg/m² or 4.5 mg/m².

In some embodiments, the amount of the compound is administered once daily, weekly or monthly.

In some embodiments, the amount of the compound is administered once daily for a three day period.

In some embodiments, the amount of the compound is administered three times per week.

In some embodiments, the amount of the compound is administered on three separate days during a seven day period.

In some embodiments, the amount of the compound is administered on three separate days during a twenty-one day treatment cycle.

In some embodiments, the amount of the compound is administered on three separate days during week 1 of a twenty-one day treatment cycle. 21 In some embodiments, the amount of the compound is administered on days 1, 2 and 3 of a twenty-one day treatment cycle.

In some embodiments, the amount of the compound is administered on days 1, 2 and 3 of a twenty-one day treatment cycle and the cycle is repeated one or more times.

In some embodiments, the amount of the compound is administered on days 1, 2 and 3 of a twenty-one day treatment cycle and the cycle is repeated one or more times.

In some embodiments, the amount of the compound is administered on days 1, 2 and 3 of a twenty-one day treatment cycle and the cycle is repeated two or more times.

In some embodiments, the amount of the compound is administered on days 1, 2 and 3 of a twenty-one day treatment cycle and the cycle is repeated three or more times.

In some embodiments, the amount of the compound is administered on days 1, 2 and 3 of a twenty-one day treatment cycle and the cycle is repeated four or more times.

In some embodiments, the amount of the compound is administered on days 1, 2 and 3 of a twenty-one day treatment cycle and the cycle is repeated five or more times.

In some embodiments, the amount of the compound is administered on days 1, 2 and 3 of a twenty-one day treatment cycle and the cycle is repeated six or more times.

In some embodiments, the amount of the compound is administered on days 1, 2 and 3 of a twenty-one day treatment cycle and the cycle is repeated between 1 to 10 times.

In some embodiments, the compound is added to an amount of normal saline (0.9%) prior to administration to the subject.

In some embodiments, the compound is added to 500 mL of normal saline (0.9%) prior to administration to the subject.

In some embodiments, the compound is administered to the subject by intravenous infusion over 1 to 3 hours.

In some embodiments, the compound is administered to the subject by intravenous infusion over 2 hours.

In some embodiments, the subject is afflicted with a cancer.

In some embodiments, the cancer is triple negative breast cancer, bladder cancer, cervical cancer, malignant mesothelioma, non-small cell lung cancer, stomach cancer, ovarian cancer, hepatocellular carcinoma, human osteosarcoma, primary liver cancer, gastric cancer, ovarian cancer, endometrial cancer, colorectal cancer, soft-tissue sarcoma, seminoma, lymphoma, fibrosarcoma, mucinous ovarian cancer, urothelial bladder cancer, squamous cell carcinoma of the uterine cervix, diffuse large cell lymphoma, lung adenoma, hepatoma, intestinal cancer, prostate cancer, angiomyolipoma, mammary adenocarcinoma, acute myelogenous leukemia, colon cancer, large cell lung cancer, adenocarcinoma of the lung, small cell lung cancer, ovary adenocarcinoma, pancreas carcinoma, prostate carcinoma, promyelocytic leukemia, chronic myelocytic leukemia, acute lymphocytic leukemia, colorectal cancer, non-Hodgkin's lymphoma, Hodgkin's lymphoma, adrenocortical cancer, osteosarcoma, esophageal, gallbladder, head and neck cancer, renal cancer, melanoma, pancreatic cancer, rectal cancer, thyroid cancer, throat cancer, glioblastoma multiforme, neuroblastoma, medulloblastoma or testicular cancer. In some embodiments, the cancer is triple negative breast cancer.

In some embodiments, the cancer is chronic myelocytic leukemia (CML).

In some embodiments, the cancer is chronic lymphocytic leukemia (CLL).

In some embodiments, the cancer is meningioma, malignant (anaplastic) meningioma, an atypical teratoid rhabdoid tumor (ATRT), a malignant rhabdoid tumor (MRT) or a diffuse intrinsic pontine glioma (DIPD).

In some embodiments, the cancer is breast, ovarian, carcinoid or testicular cancer.

In some embodiments, the cancer is pancreatic cancer.

In some embodiments, the cancer is pancreatic cancer and the cancer cells of the pancreatic cancer overexpress Mad2.

In some embodiments, wherein cells of the cancer do not overexpress N-CoR. In some embodiments, wherein cells of the cancer overexpress N-CoR. In some embodiments, wherein cells of the cancer overexpress TCTP. In some embodiments, wherein cells of the cancer overexpress Mad2.

In some embodiments, the method further comprising the administration of a chemotherapeutic agent to the human subject.

In some embodiments, the chemotherapeutic agent is a platinum-based agent or an anthracycline agent. In some embodiments, the chemotherapeutic agent is cisplatin, carboplatin, oxaliplatin, satraplatin, picoplatin, nedaplatin, triplatin, lipoplatin, doxorubicin, daunorubicin, epirubicin, idarubicin or valrubicin. In some embodiments, wherein the chemotherapeutic agent is sorafenib.

In some embodiments, the chemotherapeutic agent is x-radiation, ionizing radiation, a DNA damaging agent, a DNA intercalating agent, a microtubule stabilizing agent, a microtubule destabilizing agent, a spindle toxin, abarelix, aldesleukin, alemtuzumab, alitertinoin, allopurinol, altretamine, amifostin, anakinra, anastrozole, arsenic trioxide, asparaginase, azacitidine, bevacizumab, bexarotene, bleomycin, bortezomib, busulfan, caluaterone, capecitabine, carboplatin, carmustine, celecoxib, cetuximab, chlorambucil, cisplatin, cladribine, clofarabine, cyclophosphamide, cytarabine, dacarbazine, dactinomycin, actinomycin D, dalteparin sodium, darbepoetin alfa, dasatinib, daunorubicin, daunomycin, decitabine, denileukin, dexrazoxane, docetaxel, doxorubicin, dromostanolone propionate, exulizumab, epirubicin, epoetin alfa, erlotinib, estramustine, etoposide phosphate, etoposide, VP-16, exemestane, fentanyl citrate, filgrastim, floxuridine, fludarabine, fluorouracil, fulvestrant, gefitinib, gemcitabine, gosereline acetate, histrelin acetate, hydroxyurea, ibritumomab tiuxetan, idarubicin, ifosfamide, imatinib mesylate, interferon alfa 2a, interferon alfa 2b, irinotecan, lapatinib ditosylate, lenalidomide, letrozole, leucovrin, leuprolide acetate, levamisole, lomustine, meclorethamine, megestrol acetate, melphalan, mercaptopurine, mesna, methotrexate, methoxsalen, mitomycin C, mitotane, mitoxantrone, nandrolone phenpropionate, nelarabine, nofetumomab, nitrosoureas, oprelvekin, oxaliplatin, paclitaxel, palifermin, pamidronate, panitumumab, pegademase, pegaspargase, pegfilgrastim, peginterferon alfa 2b, pemetrexed disodium, pentostatin, pipobroman, plicamycin, mithramycin, porfimer sodium, procarbazine, quinacrine, rasburicase, rituximab, sargrmostim, sorafenib, streptozocin, sunitinib, sunitinib maleate, talc, tamoxifen, temozolomide, teniposide, VM-26, testolactone, thalidomide, thioguanine, G-TG, thiotepa, topotecan, toremifene, tositumomab, trastuzumab, tretinoin ATRA, ruacil mustard, valrunicin, vinblastine, vincristine, vinorelbine, vorinostat, zoledronate, or zoledronic acid.

In some embodiments, the chemotherapeutic agent is x-radiation or ionizing radiation. In some embodiments, the chemotherapeutic agent is docetaxel.

In some embodiments, the docetaxel Is administered to the subject in an amount of 55 mg/m² to 80 mg/m².

In some embodiments, the docetaxel is administered to the subject in an amount of about 55 mg/m², 60 mg/m², 65 mg/m², 70 mg/m² or 75 mg/m².

In some embodiments, the amount of the chemotherapeutic agent is administered once daily.

In some embodiments, the amount of the chemotherapeutic agent is administered once during a three day period.

In some embodiments, the amount of the chemotherapeutic agent is administered one time per week.

In some embodiments, the amount of the chemotherapeutic agent is administered on a single day during a seven day period.

In some embodiments, the amount of the chemotherapeutic agent is administered on a single day of a twenty-one day treatment cycle.

In some embodiments, the amount of the chemotherapeutic agent is administered on a single day during week 1 of a twenty-one day treatment cycle.

In some embodiments, the amount of the chemotherapeutic agent is administered on day 2 of a twenty-one day treatment cycle.

In some embodiments, the amount of the compound is administered on days 1, 2 and 3 and the amount of the chemotherapeutic agent is administered on day 2 of a twenty-one day treatment cycle.

In some embodiments, the amount of the compound is administered on days 1, 2 and 3 and the amount of the chemotherapeutic agent is administered on day 2 of a twenty-one day treatment cycle and the cycle is repeated one or more times, two or more times, three or more times, four or more time, five or more times, or 6 or more times.

In some embodiments, the amount of the compound is administered on days 1, 2 and 3 and the amount of the chemotherapeutic agent is administered on day 2 of a twenty-one day treatment cycle and the cycle is repeated from 1 to 10 times.

In some embodiments, the amount of the compound and the amount of the chemotherapeutic agent are administered simultaneously, separately or sequentially.

In some embodiments, the amount of the compound and the amount of the chemotherapeutic agent when taken together is more effective to treat the subject than when the chemotherapeutic agent is administered alone.

In some embodiments, the amount of the compound and the amount of the chemotherapeutic agent when taken together is effective to reduce a clinical symptom of the cancer in the subject.

In some embodiments, the treating comprises inhibiting proliferation of or inducing apoptosis of cancer cells in the subject.

In some embodiments, the compound enhances the chemotherapeutic effect of the chemotherapeutic agent.

In some embodiments, the compound chemosensitizes the cancer to the chemotherapeutic agent.

In some embodiments, the compound increases chemosensitization of the cancer to the chemotherapeutic agent.

In some embodiments, the compound is administered 5 times per week and the radiation is administered 5 times per week for 1 or more weeks.

In some embodiments, the amount of the compound administered is 1.5 mg/m² to 3.0 mg/m².

In some embodiments, the amount of the compound and the radiation are administered on the same 5 days.

In some embodiments, the amount of the compound and the radiation are administered on days 1-5 of each week.

In some embodiments, the amount of the compound is administered 3 times per week and the radiation is administered 5 times per week for 1 or more weeks.

In some embodiments, the amount of the compound administered is 1.5 mg/m² to 4.5 mg/m².

In some embodiments, the amount of the compound is administered on days 1, 3 and 5 of each week and the radiation is administered on days 1-5 of each week.

In some embodiments, the amount of the compound is administered 2 hours after the administration of the radiation.

In some embodiments, the amount of the compound administered is 1.5 mg/m² to 4.5 mg/m².

In some embodiments, the amount of the compound is administered on days 1 and 2 of a two day cycle and the radiation is administered on day 2 of the two day cycle and the cycle is repeated 1 or more times.

In some embodiments, the amount of the compound administered is 1.5 mg/m² to 4.5 mg/m².

In some embodiments, the chemotherapeutic agent is doxorubicin and is administered to the subject in an amount from 40 mg/m² to 60 mg/m² once every three weeks or every four weeks or an amount from 60 mg/m² to 75 mg/m² once every three weeks.

In some embodiments, the chemotherapeutic agent is cisplatin and is administered to the subject in an amount from 60 mg/m² to 100 mg/m² once every three weeks or every four weeks.

In some embodiments, the chemotherapeutic agent is temozolomide and is administered to the subject in an amount from 150 mg/m² (initial dose) to 200 mg/m² (maintenance dose) once daily orally.

In some embodiments, the chemotherapeutic agent is temozolomide and is administered to the subject in an amount from 150 mg/m² (initial dose) to 200 mg/m² (maintenance dose) once daily for the first 5 days of a 28 day cycle.

In some embodiments, the chemotherapeutic agent is temozolomide and is administered to the subject in an amount from 150 mg/m² (initial dose) to 200 mg/m² (maintenance dose) once daily over ninety minutes by IV.

In some embodiments, the chemotherapeutic agent is temozolomide and is administered to the subject in an amount from 150 mg/m² (initial dose) to 200 mg/m² (maintenance dose) once daily over ninety minutes by IV for the first 5 days of a 28 day cycle.

In some embodiments, the subject is afflicted with a reperfusion injury.

In some embodiments, the treating of the reperfusion injury comprises reducing reperfusion injury in tissue in the subject.

In some embodiments, the tissue in the subject is myocardial tissue, brain tissue or endothelial tissue

In some embodiments, the reperfusion injury is caused by an ischemia.

In some embodiments, the ischemia is caused by myocardial infarction, stroke or sepsis.

In some embodiments, the treating of the reperfusion injury comprises reducing tissue damage associated with reperfusion injury in the heart of the subject following a myocardial infarction.

In some embodiments, the treating of the reperfusion injury comprises reducing vascular leakage associated with reperfusion injury in the subject suffering from sepsis.

In some embodiments, the treating of the reperfusion injury comprises reducing tissue damage due to an acute trauma in the subject or reducing vascular leakage due to an acute trauma in the subject.

In some embodiments, the subject is afflicted with a disease characterized by a loss of protein function caused by a genetic abnormality associated with the disease.

In some embodiments, the disease characterized by a loss of protein function is Gaucher's disease, von Hippel-Lindau disease, cystic fibrosis, Phenylketonuria, Fabry disease, Tay-Sachs disease, Pompe disease, Neimann-Pick disease (Type A, B and C), Marfan syndrome, Hemophilia A & B, retinitis pigmentosa, Neurofibromatosis Type 2, pheochromocytoma, paraganglioma, Multiple Endocrine Neoplasia Type 1, Familial Hypercholesterolemia, Hurler's disease, Hunter syndrome, Sanfilippo syndrome, Morquio syndrome, Maroteaux-Lamy syndrome, Sly syndrome, Sandhoff's disease, Fucosidosis, alpha-mannosidosis, beta-mannosidosis, aspartylglucosaminuria, Sialidosis, Inclusion-cell (I-cell) disease, Pseudo-Hurler polydystrophy, Krabbe's disease, Metachromatic leukodystrophy, multiple sulfatase deficiency, Wolmen's disease, Cholesteryl ester storage disease, Late onset GAA deficiency, Danon's disease, Neutropenia, X-linked hyper IgM syndrome, X-linked agammaglobulinemia, X-linked lymphoproliferative disease, Severe Combined Immunodeficiency, Noonan syndrome, juvenile myelomonocytic leukemia, Basal cell carcinoma, STAT1 deficiency, Alzheimer's disease, Parkinson's disease, Huntington's disease, TTR Amyloid Polyneuropathy, Ataxia Telangiectasia, Creutzfeldt-Jakob disease, Type II diabetes, Hereditary Transthyretin (TTR) amyloidosis, pheochromocytomas (PHEO) or paragangliomas (PGL).

In some embodiments, the disease characterized by a loss of protein function is Gaucher's disease.

In some embodiments, the subject is afflicted with Type 2 Diabetes.

In some embodiments, the treating of the Type 2 Diabetes comprises increasing the insulin sensitivity of cells in the subject.

In some embodiments, the cells are liver, fat, muscle or endothelial cells.

In some embodiments, the treating comprises reducing complications associated with Type 2 Diabetes in the subject.

In some embodiments, the disease or condition mediated by the normal expression, overexpression, or under expression of protein phosphatase 2A (PP2A) is a neurodegenerative disease.

In some embodiments, the disease or condition mediated by the normal expression, overexpression, or under expression of protein phosphatase 2A (PP2A) is a neurodegenerative disease mediated by under expression of protein phosphatase 2A (PP2A).

In some embodiments, the neurodegenerative disease is diabetic neuropathy, senile dementias, Alzheimer's disease, Mild Cognitive Impairment (MCI), dementia, Lewy Body Dementia, Frontal Temporal Lobe dementia (Pick's disease), Parkinson's Disease, facial nerve (Bell's) palsy, glaucoma, Huntington's chorea, amyotrophic lateral sclerosis (ALS), status epilepticus, non-arteritic optic neuropathy, intervertebral disc herniation, vitamin deficiency, Creutzfeldt-lakob disease, carpal tunnel syndrome, peripheral neuropathies, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, Charcot-Marie-Tooth disease, ataxia telangectasia, Friedreich's ataxia, amyloid polyneuropathies, adrenomyeloneuropathy, Giant axonal neuropathy, Refsum's disease, Fabry's disease and lipoproteinemia, Progressive Supranuclear Palsy or Corticobasal degeneration.

In some embodiments, the method comprising administering to the subject an amount of 0.25 mg to 7.5 mg of LB-100.

In some embodiments, the method comprising administering to the subject an amount of 0.25 mg to 4 mg of LB-100.

In some embodiments, the method comprising administering to the subject an amount of 0.25 mg to 3 mg of LB-100.

In some embodiments, the method comprising administering to the subject an amount of 0.25 mg to 2 mg of LB-100.

In some embodiments, the method comprising administering to the subject an amount of 0.25 mg to 1 mg of LB-100.

In some embodiments, the method comprising administering to the subject an amount of 1 mg to 5 mg of LB-100.

In some embodiments, the method comprising administering to the subject an amount of 1 mg to 4 mg of LB-100.

In some embodiments, the method comprising administering to the subject an amount of 1 mg to 3 mg of LB-100.

In some embodiments, the method comprising administering to the subject an amount of 1 mg to 2 mg of LB-100.

In some embodiments, the method comprising administering to the subject an amount of 4 mg to 7.5 mg of LB-100.

In some embodiments, the method comprising administering to the subject an amount of 4 mg to 5 mg of LB-100.

In some embodiments, the method comprising administering to the subject an amount of 5 mg to 6 mg of LB-100.

In some embodiments, the method comprising administering to the subject an amount of 6 mg to 7 mg of LB-100.

In some embodiments, the method comprising administering to the subject an amount of 7 mg to 7.5 mg of LB-100.

In some embodiments, the method comprising administering to the subject an amount of 4.8 mg to 7.2 mg of LB-100.

In some embodiments, the method comprising administering to the subject an amount of about 0.4 mg, 0.8 mg, 1.3 mg, 2 mg, 2.8 mg or 3.7 mg of LB-100.

In some embodiments, the method comprising administering to the subject an amount of about 0.25 mg, 0.5 mg, 0.75 mg, 1 mg, 1.25 mg, 1.5 mg, 1.75 mg, 2 mg, 2.25 mg, 2.5 mg, 2.75 mg, 3 mg, 3.25 mg, 3.5 mg, 3.75 mg, 4 mg, 4.25 mg, 4.5 mg, 4.75 mg, 5 mg, 5.25 m, 5.5 mg, 5.75 mg, 6 mg, 6.25 mg, 6.5 mg, 6.75 mg, 7 mg, 7.25 mg or 7.5 mg of LB-100.

In some embodiments, the method further comprising administering to the subject docetaxel in an amount of 75 mg to 150 mg.

In some embodiments, the method further comprising administering to the subject docetaxel in an amount of 90 mg to 125 mg.

In some embodiments, the method further comprising administering to the subject docetaxel in amount of 90 mg to 100 mg.

In some embodiments, the method further comprising administering to the subject docetaxel in amount of 115 mg to 125 mg.

In some embodiments, the method further comprising administering to the subject docetaxel in amount of about 90 mg, 95 mg, 100 mg, 115 mg, 120 mg or 125 mg.

The present invention provides a method of treating a human subject afflicted with chronic myelogenous leukemia (CML) comprising administering to the subject an amount of from 0.25 mg to 7.5 mg or an amount from 0.1 mg/m² to 5.0 mg/m² of LB100 so as to thereby treat the CML in the subject.

In some embodiments of the above method of treating CML, further comprising administering to the subject an amount of dasatinib, imatinib or imatinib mesylate.

The present invention provides a method of treating triple-negative breast cancer in a human subject afflicted therewith comprising administering to the subject an amount of from 0.25 mg to 7.5 mg or an amount from 0.1 mg/m² to 5.0 mg/m² of LB100 so as to thereby treat the triple-negative breast cancer in the subject.

The present invention provides a method of treating bladder cancer, cervical cancer, malignant mesothelioma, non-small cell lung cancer, stomach cancer or ovarian cancer in a human subject afflicted therewith comprising administering to the subject an amount of from 0.25 mg to 7.5 mg or an amount from 0.1 mg/m² to 5.0 mg/m² of LB100 so as to thereby treat the bladder cancer, cervical cancer, malignant mesothelioma, non-small cell lung cancer, stomach cancer or ovarian cancer in the subject.

The present invention provides a method of treating cancer in a human subject afflicted therewith which comprises the following:

(a) determining the levels of Mad2 in the cancers cells isolated from the subject;
(b) comparing the levels of Mad2 in the cancers cells relative to a predetermined reference level of Mad2; and
(c) administering an amount of from 0.25 mg to 7.5 g or an amount from 0.1 mg/m² to 5.0 mg/m² of LB100 to the subject if there are higher levels of Mad2 in the cancer cells as compared with the predetermined reference level of Mad2.

In some embodiments, the above method further comprising

(d) administering an effective amount of a chemotherapeutic agent to the subject if there are higher levels of Mad2 in the cancer cells as compared with the predetermined reference level of Mad2.

In some embodiments, the chemotherapeutic agent is an HDAC inhibitor.

In some embodiments, the chemotherapeutic agent is an HDAC inhibitor selected from 2-amino-8-oxo-9,10-epoxy-decanoyl, 3-(4-aroyl-1H-pyrrol-2-yl)-N-hydroxy-2-propenamide, APHA Compound 8, apicidin, arginine butyrate, butyric acid, depsipeptide, depudecin, HDAC-3 inhibitor, m-carboxycinnamic acid bis-hydroxamide, N-(2-aminophenyl)-4-[N-(pyridin-3-ylmethoxycarbonyl)aminomethyl)benzamide, MS 275, oxamfiatin, phenylbutyrate, pyroxamide, scriptaid, sirtinol, sodium butyrate, suberic bishydroxamic acid, suberoylanilide hydroxamic acid, trichostatinA, trapoxinA, trapoxin B, and valproic acid.

In some embodiments, the chemotherapeutic agent is a retinoid receptor ligand. In some embodiments, the chemotherapeutic agent is a retinoid receptor ligand selected from b,g selective 6-(5,6,7,S-tetrahydro-5,5,S,S-tetramethyl-2-naphthalenyl)-2-naph-thalenecarboxylic acid (TTNN), Z-oxime of 6-(5,6,7,S-tetrahydro-5,5,S,S-tetramethyl-2-naphthalenylcarbonyl)-2-naphthalenecarboxylic acid (SR11254), 4-(5,6,7, S-tetrahydro-5,5,S,S-tetramethyl-2-anthracenyl)benzoic, acid (TTAB), 4-[1-(5,6,7,8-tetrahydro-5,5,8,8-tetramethyl-2-naphthalenyl)-cyclopropyl]benzoic acid (SR11246), 4-[1-(5,6,7,8-tetrahydro-3,5,5,8,8-pentamethyl-2-naphthalenyl)-2-methylpropenyl]benzoic acid (SR11345), and 2-(6-carboxy-2-naphthalenyl)-2-(5,6,7,8-tetrahydro-5,5,8,8-tetramethyl-2-naphthalenyl)-1,3-dithiolane (SRI1253), ATRA, vitamin A (retinol) and all its natural and synthetic derivatives (retinoids).

The present invention also provides a method of treating cancer in a subject afflicted therewith which comprises the following:

(a) determining the levels of Mad2 in the cancers cells isolated from the subject;
(b) comparing the levels of Mad2 in the cancers cells relative to a predetermined reference level of Mad2; and
(c) administering an effective amount of a compound to the subject if there are higher levels of Mad2 in the cancer cells as compared with the predetermined reference level of Mad2.

In some embodiments, the method further comprising

(d) administering an effective amount of an anti-cancer agent to the subject if there are higher levels of Mad2 in the cancer cells as compared with the predetermined reference level of Mad2.

In some embodiments, the method wherein the compound is a PP2A inhibitor.

In some embodiments, the method wherein the compound is a PP2A inhibitor having the structure:

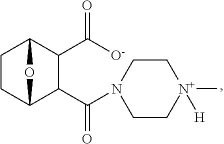 [SMILES: C[NH+]1CCN(C(=O)C2C(C(=O)[O-])[C@H]3CC[C@@H]2O3)CC1]

or a salt or ester thereof.

The present invention also provides a method of predicting whether a subject afflicted with cancer is likely to exhibit a positive clinical response to treatment with a PP2A inhibitor which comprises the following:

(a) determining the levels of Mad2 in cancers cells isolated from the subject; and
(b) comparing the levels of Mad2 in the cancers cells relative to a predetermined reference level of Mad2,
wherein higher levels of Mad2 in the cancer cells as compared with the predetermined reference level of Mad2 indicates that the subject is likely to exhibit a positive clinical response to treatment with the PP2A inhibitor.

In some embodiments, a method of treating a Mad2-overexpressing cancer in a subject afflicted therewith comprising administering to the subject an effective amount of a PP2A inhibitor of the present invention.

In some embodiments, a method of treating a Mad2-overexpressing cancer in a subject afflicted therewith comprising administering to the subject an effective amount of an anti-cancer agent and an effective amount of a PP2A inhibitor of the present invention.

In some embodiments, the amount of the compound is effective to reduce a clinical symptom of the cancer in the subject.

In some embodiments, the treating comprises inhibiting proliferation of cancer cells in the subject, inducing apoptosis of cancer cells in the subject, or reducing the size of a tumor in the subject.

In some embodiments, the compound reduces the levels of Mad2 in the cancer cells in the subject.

In some embodiments, the compound inhibits the growth of Mad2-overexpressing cancer cells in the subject.

In some embodiments, the compound induces mitotic delay or mitotic arrest of Mad2-overexpressing cancer cells in the subject.

In some embodiments, the amount of the compound and the amount of the anti-cancer agent are each periodically administered to the subject

In some embodiments, the amount of the compound and the amount of the anti-cancer agent are administered simultaneously, separately or sequentially.

In some embodiments, the amount of the compound and the amount of the anti-cancer agent when taken together is more effective to treat the subject than when the anti-cancer agent is administered alone.

In some embodiments, the amount of the compound and the amount of the anti-cancer agent when taken together is effective to reduce a clinical symptom of the cancer in the subject.

In some embodiments, the treating comprises inhibiting proliferation of or inducing apoptosis of the cancer cells in the subject.

In some embodiments, the anti-cancer agent is a chemotherapeutic agent.

In some embodiments, the anti-cancer agent is x-radiation or ionizing radiation.

In some embodiments, the anti-cancer agent is selected from x-radiation, ionizing radiation, a DNA damaging agent, a DNA intercalating agent, a microtubule stabilizing agent, a microtubule destabilizing agent, a spindle toxin, abarelix, aldesleukin, alemtuzumab, alitertinoin, allopurinol, altretamine, amifostin, anakinra, anastrozole, arsenic trioxide, asparaginase, azacitidine, bevacizumab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, dacarbazine, dactinomycin, actinomycin D, dalteparin sodium, darbepoetin alfa, dasatinib, daunorubicin, daunomycin, decitabine, denileukin, dexrazoxane, docetaxel, doxorubicin, dromostanolone propionate, exulizumab, epirubicin, epoetin alfa, erlotinib, estramustine, etoposide phosphate, etoposide, VP-16, exemestane, fentanyl citrate, filgrastim, floxuridine, fludarabine, fluorouracil, fulvestrant, gefitinib, gemcitabine, gosereline acetate, histrelin acetate, hydroxyurea, ibritumomab tiuxetan, idarubicin, ifosfamide, imatinib mesylate, interferon alfa 2a, interferon alfa 2b, irinotecan, lapatinib ditosylate, lenalidomide, letrozole, leucovrin, leuprolide acetate, levamisole, lomustine, meclorethamine, megestrol acetate, melphalan, mercaptopurine, mesna, methotrexate, methoxsalen, mitomycin C, mitotane, mitoxantrone, nandrolone phenpropionate, nelarabine, nofetumomab, oprelvekin, oxaliplatin, paclitaxel, palifermin, pamidronate, panitumumab, pegademase, pegaspargase, pegfilgrastim, peginterferon alfa 2b, pemetrexed disodium, pentostatin, pipobroman, plicamycin, mithramycin, porfimer sodium, procarbazine, quinacrine, rasburicase, rituximab, sargrmostim, sorafenib, streptozocin, sunitinib, sunitinib maleate, talc, tamoxifen, temozolomide, teniposide, VM-26, testolactone, thalidomide, thioguanine, G-TG, thiotepa, topotecan, toremifene, tositumomab, trastuzumab, tretinoin ATRA, ruacil mustard, valrunicin, vinblastine, vincristine, vinorelbine, vorinostat, zoledronate, and zoledronic acid.

In some embodiments, the anti-cancer agent is a platinum-based anti-cancer agent or an anthracycline anti-cancer agent.

In some embodiments, the anti-cancer agent is docetaxel, cisplatin, carboplatin, oxaliplatin, satraplatin, picoplatin, nedaplatin, triplatin, lipoplatin, doxorubicin, daunorubicin, epirubicin, idarubicin, or valrubicin.

In some embodiments, the cancer overexpressing Mad-2 is breast cancer, colon cancer, large cell lung cancer, adenocarcinoma of the lung, small cell lung cancer, stomach cancer, liver cancer, ovary adenocarcinoma, pancreas carcinoma, prostate carcinoma, promylocytic leukemia, chronic myelocytic leukemia, acute lymphocytic leukemia, colorectal cancer, ovarian cancer, lymphoma, non-Hodgkin's lymphoma or Hodgkin's lymphoma.

In some embodiments, the cancer overexpressing Mad-2 is hepatocellular carcinoma, human osteosarcoma, primary liver cancer, gastric cancer, ovarian cancer, endometrial cancer, colorectal cancer, non-small cell lung cancer, soft-tissue sarcoma, seminoma, breast cancer, lymphoma, fibrosarcoma, neuroblastoma, mucinous ovarian cancer, urothelial bladder cancer, squamous cell carcinoma of the uterine cervix, diffuse large cell lymphoma, lung adenoma, hepatoma, intestinal cancer, fibrosarcoma, prostate cancer, angiomyolipoma, mammary adenocarcinoma or acute myelogenous leukemia.

In some embodiments, the cancer overexpressing Mad-2 is hepatocellular carcinoma, human osteosarcoma, primary liver cancer, gastric cancer, ovarian cancer, endometrial cancer, colorectal cancer, non-small cell lung cancer, soft-tissue sarcoma, seminoma, breast cancer, lymphoma, fibrosarcoma, or neuroblastoma.

In some embodiments, the cancer overexpressing Mad-2 is mucinous ovarian cancer, urothelial bladder cancer, squamous cell carcinoma of the uterine cervix, or diffuse large cell lymphoma.

In some embodiments, the cancer overexpressing Mad-2 is lung adenoma, hepatoma, hepatocellular carcinoma, intestinal cancer, lymphoma, fibrosarcoma, prostate cancer, angiomyolipoma, or mammary adenocarcinoma.

In some embodiments, the cancer is acute myelogenous leukemia.

In some embodiments, the cancer transiently overexpresses Mad2.

In some embodiments, the cancer permanently overexpresses Mad2.

In some embodiments, the cancer is advanced or has metastasized or has not gotten better with other types of treatment or chemotherapy.

In some embodiments, the cancer is advanced and/or cannot be treated with surgery or radiation therapy.

In some embodiments, the subject afflicted with cancer has already had surgery or radiation therapy.

In some embodiments, the cancer is refractory.

In some embodiments, the “predetermined reference level of Mad2” refers to an average level of Mad2 expression in non-cancer cells isolated from the subject.

In some embodiments, the “predetermined reference level of Mad2” refers to an average level of Mad2 expression in adjacent non-tumorous cells isolated from the subject.

In some embodiments, the “predetermined reference level of Mad2” refers to an average level of Mad2 expression in non-cancer cells isolated from a control group of subjects not afflicted with cancer.

In some embodiments, the “predetermined reference level of Mad2” value is determined by analyzing the expression levels of Mad2 in cancer cells isolated from a control group of subjects afflicted with cancer.

In some embodiments, the “predetermined reference level of Mad2” value is determined by analyzing the expression levels of Mad2 in adjacent non-tumorous cells isolated from a control group of subjects afflicted with cancer.

In some embodiments, the levels of Mad2 in the cancer cells in the subject are greater than or equal to 1.1 times the predetermined reference level of Mad2.

In some embodiments, the levels of Mad2 in the cancer cells in the subject are greater than or equal to 1.5 times the predetermined reference level of Mad2.

In some embodiments, the levels of Mad2 in the cancer cells in the subject are greater than or equal to 5.0 times the predetermined reference level of Mad2.

In some embodiments, the levels of Mad2 in the cancer cells in the subject are greater than or equal to 10.0 times the predetermined reference level of Mad2.

The present invention provides a method of inhibiting protein phosphatase 2A (PP2A) in a human subject in need thereof comprising administering to the subject an amount of from 0.1 mg/m² to 5 mg/m² of any of the below compounds, or a salt, zwitterion, or ester thereof, so as to thereby inhibit protein phosphatase 2A (PP2A) in the subject.

The present invention provides a method of inhibiting protein phosphatase 2A (PP2A) in a human subject in need thereof comprising administering to the subject an amount of from 0.1 mg/m² to 5 mg/m² of any of the compounds disclosed herein, or a salt, zwitterion, or ester thereof, so as to thereby inhibit protein phosphatase 2A (PP2A) in the subject.

In some embodiments of any of the above methods, the compound has the structure:

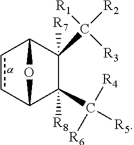

In some embodiments of the method, bond α in the compound is present.

In some embodiments of the method, bond α in the compound is absent.

In some embodiments of the method, the compound wherein
R₃ is OH, O⁻, or OR₉,
   wherein R, is alkyl, alkenyl, alkynyl or aryl;
R₄ is

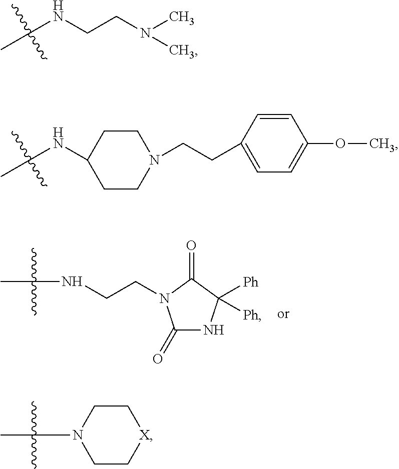

where X is O, S, NR₁₀, N′HR₁₀ or N′R₁₀R₁₀,
   where each R₁₀ is independently H, alkyl, alkenyl, alkynyl, aryl,

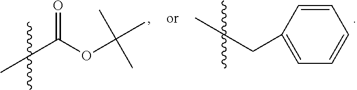

In some embodiments of the method, the compound wherein
R₃ is OH, O⁻ or OR₉,
   where R₉ is H, methyl, ethyl or phenyl.

In some embodiments of the method, the compound wherein
R₃ is OH, O⁻ or OR₉,
   wherein R₉ is methyl.

In some embodiments of the method, the compound wherein
R₄ is

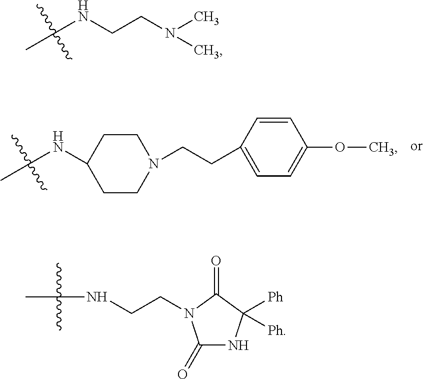

In some embodiments of the method, the compound wherein
R₄ is

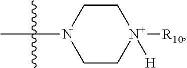

wherein R₁₀ is H, alkyl, alkenyl, alkynyl, aryl, or

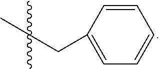 [SMILES: *Cc1ccccc1]

In some embodiments of the method, the compound wherein
R₄ is

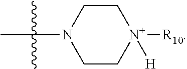

wherein R₁₀ is —H, —CH₃, —CH₂CH₃, or

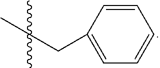 [SMILES: *Cc1ccccc1]

In some embodiments of the method, the compound wherein
R₄ is

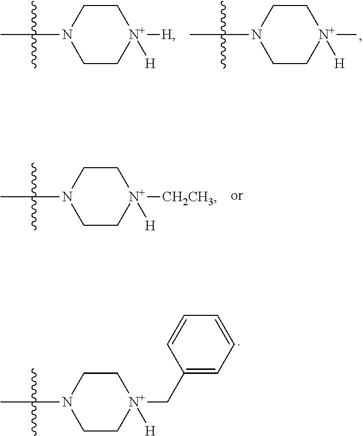

In some embodiments of the method, the compound wherein
R₄ is

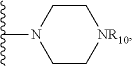

wherein R₁₀ is H, alkyl, alkenyl, alkynyl, aryl,

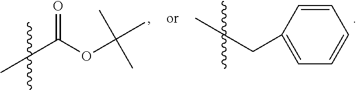

In some embodiments of the method, the compound wherein
R₄ is

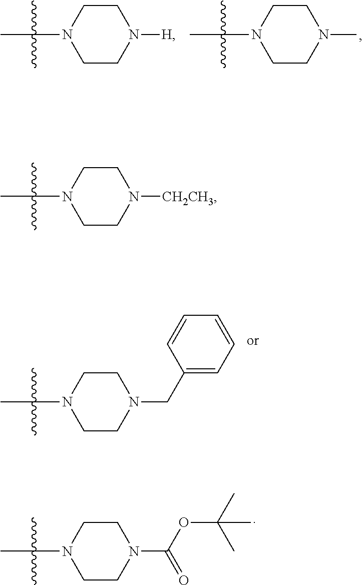

In some embodiments of the method, the compound wherein
R₄ is

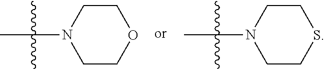 [SMILES: *N1CCOCC1 ; "or" ; *N1CCSCC1]

In some embodiments of the method, the compound has the structure:

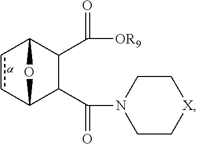

wherein
bond α is present or absent;
R₉ is present or absent and when present is H, alkyl, alkenyl, alkynyl or phenyl; and
X is O, NR₁₀, NH⁺R₁₀ or N′R₁₀R₁₀,
   where each R₁₀ is independently H, alkyl, substituted alkyl, alkenyl, substituted alkenyl, alkynyl, substituted alkynyl, aryl,

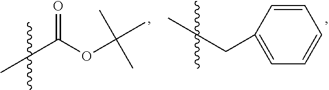

—CH₂CN, —CH₂CO₂R₁₂, or —CH₂COR₁₂.
   where R₁₂ is H or alkyl,
      or a salt, zwitterion or ester thereof.

In some embodiments of the method, the compound has the structure:

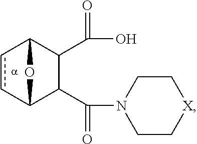

wherein
bond α is present or absent;
X is O or NR₁₀,
   where each R₁₀ is independently H, alkyl, substituted alkyl, alkenyl, substituted alkenyl, alkynyl, substituted alkynyl, aryl,

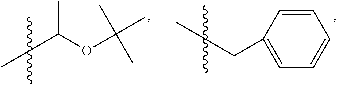

—CH₂CN, —CH₂CO₂R₁₂, or —CH₂COR₁₂.
   where R₁₂ is H or alkyl,
      or a salt, zwitterion or ester thereof.

In some embodiments of the method, the compound has the structure:

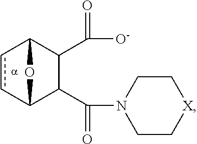

wherein
bond α is present or absent;
X is O or NH⁺R₁₀,
   where R₁₀ is H, alkyl, substituted alkyl, alkenyl, substituted alkenyl, alkynyl, substituted alkynyl, aryl,

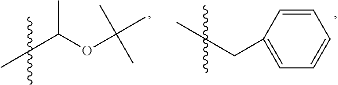

—CH₂CN, —CH₂CO₂R₁₂, or —CH₂COR₁₂.
   where R₁₂ is H or alkyl,
      or a salt, zwitterion or ester thereof.

In some embodiments of the method, the compound has the structure:

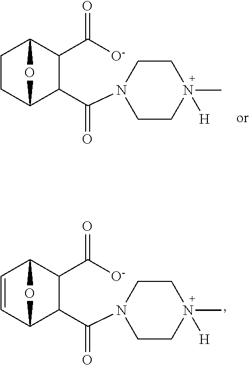

or a salt or ester thereof.

In one embodiment, the compound of the method has structure:

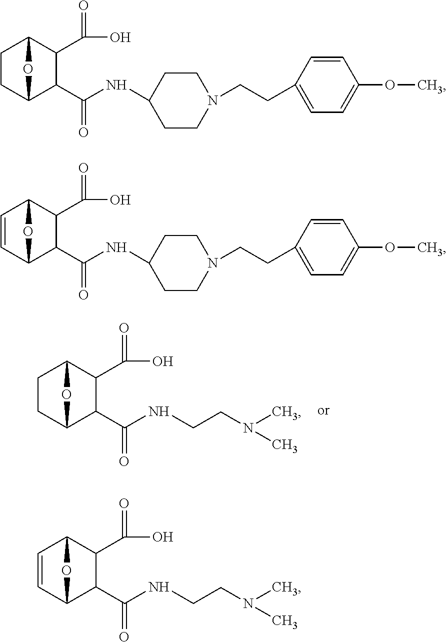

or a salt, zwitterion, or ester thereof.

In one embodiment, the compound of the method has the structure:

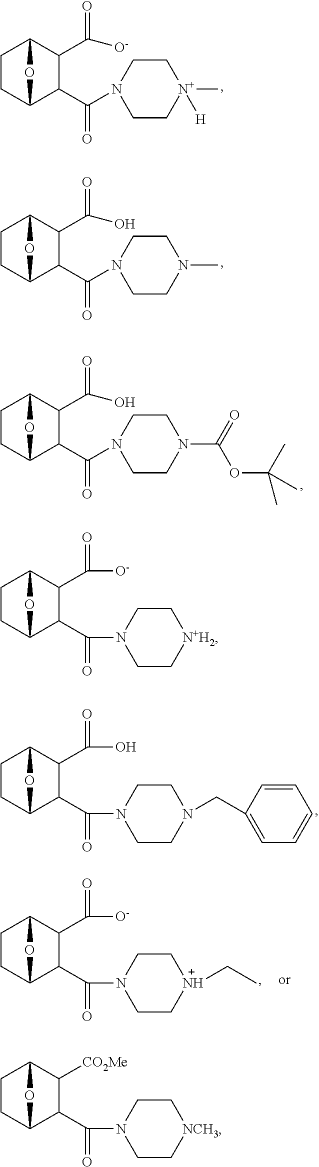

or a salt, zwitterion, or eater thereof.

In one embodiment the compound of the method has the structure:

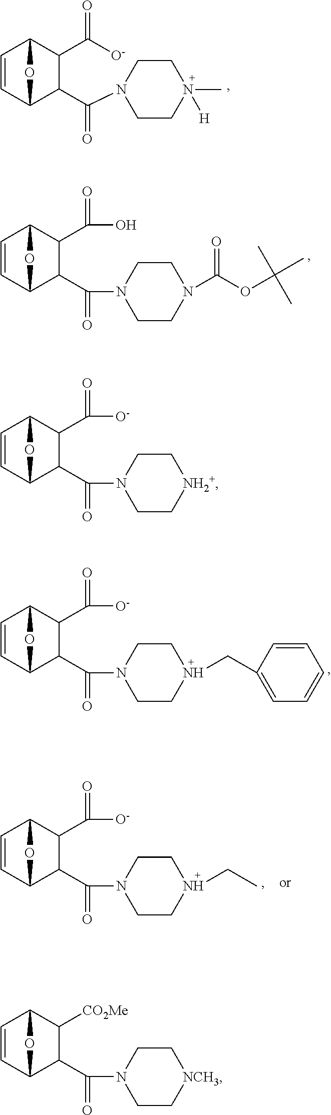

or a salt, zwitterion, or ester thereof.

In one embodiment, the compound of the method has the structure:

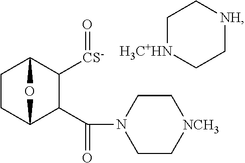

or a salt, zwitterion, or ester thereof.

In some embodiments of the method, the compound having the structure:

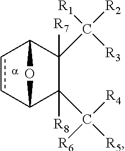

wherein
bond α is present or absent;
R₁ and R₂ together are ═O;
R₃ and R₄ are each different, and each is O(CH₂)₁₋₆R₉ or OR₉, or

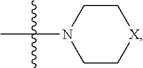

where X is O, S, NR₁₀, N⁺HR₁₀ or N⁺R₁₀R₁₀,
   where each R₉ is H, alkyl, C₂-C₁₂ alkyl substituted alkyl, alkenyl, alkynyl, aryl, (C₆H₅)(CH₂)₁₋₆(CHNHBOC)CO₂H, (C₆H₅)(CH₂)₁₋₆(CHNH₂) CO₂H, (CH₂)₁₋₆(CHNHBOC)CO₂H, (CH₂)₁₋₄(CHNH₂)CO₂H or (CH₂)₁₋₆CCl₃,
   where each R₁₀ is independently H, alkyl, hydroxyalkyl, C₂-C₁₂ alkyl, alkenyl, C₄-C₁₂ alkenyl, alkynyl, aryl,

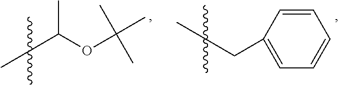

—CH₂CN, —CH₂CO₂R₁₁, or —CH₂COR₁₁.
   where each R₁₁ is independently alkyl, alkenyl or alkynyl, each of which is substituted or unsubstituted, or H;
or R₃ and R₄ are each different and each is OH or

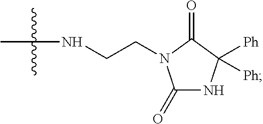 [SMILES: *NCCN1C(=O)NC(c2ccccc2)(c2ccccc2)C1=O]

R₅ and R₄ taken together are —O;
R₇ and R₈ are each H; and
each occurrence of alkyl, alkenyl, or alkynyl is branched or unbranched, unsubstituted or substituted,
   or a salt, zwitterion, or ester thereof.

In one embodiment, the compound of the method has the structure:

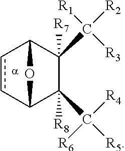

In one embodiment of the method, the bond α is present.

In one embodiment of the method, the bond α is absent.

In one embodiment of the method,

R₃ is OR₉ or O(CH₂)₁₋₆R₉,
where R₉ is aryl, substituted ethyl or substituted phenyl,
wherein the substituent is in the pare position of the phenyl;

### R₄ is

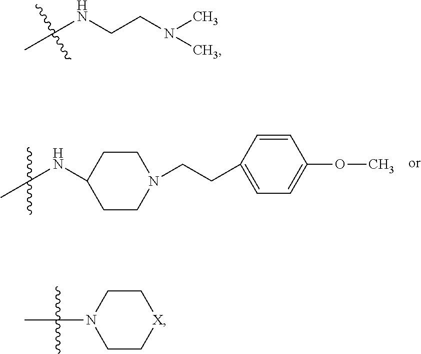 [0277] where X is O, S, NR₁₀, or N⁺R₁₀R₁₀,
where each R₁₀ is independently H, alkyl, hydroxyalkyl, substituted C₂-C₁₂ alkyl, alkenyl, substituted C₄-C₁₂ alkenyl, alkynyl, substituted alkynyl, aryl,

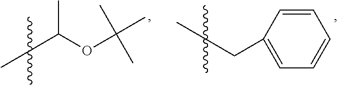

—CH₂CN, —CH₂CO₂R₁₁, —CH₂COR₁₁,
   where R₁₁ is alkyl, alkenyl or alkynyl, each of which is substituted or unsubstituted, or H;
      or where R₃ is OH and R₄ is

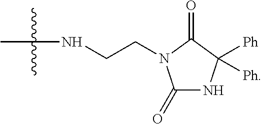 [SMILES: *NCCN1C(=O)NC(c2ccccc2)(c2ccccc2)C1=O]

In one embodiment of the method,

### R₄ is

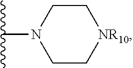 [0283] where R₁₀ is alkyl or hydroxylalkyl.

In one embodiment of the method,
R₁ and R₂ together are ═O;
R₃ is OR₉ or O(CH₂)₁₋₂R₉,
   where R₉ is aryl, substituted ethyl, or substituted phenyl,
   wherein the substituent is in the para position of the phenyl;
R₄ is

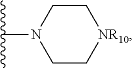

where R₁₀ is alkyl or hydroxyl alkyl;
R₅ and R₆ together are ═O; and
R₇ and R₈ are each independently H.

In one embodiment of the method,
R₁ and R₂ together are ═O;
R₃ is O(CH₂)R₉, or OR₉,
   where R₉ is phenyl or CH₂CCl₃,

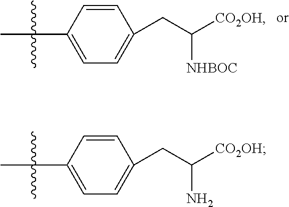

R₄ is

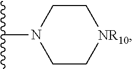

where R₁₀ is CH₃ or CH₃CH₂OH;
R₅ and R₆ together are ═O; and
R₇ and R₈ are each independently H.

In one embodiment of the method,
R₃ is OR₉,
   where R₉ is (CH₂)₁₋₆(CHNHBOC)CO₂H, (CH₂)₁₋₆(CHNH₂)CO₂H, or (CH₂)₁₋₆CCl₃.

In one embodiment of the method,
R₉ is CH₂(CHNHBOC)CO₂H, CH₂(CHNH₂)CO₂H, or CH₂CCl₃.

In one embodiment of the method,
R₉ is (C₆H₅)(CH₂)₁₋₆(CHNHBOC)CO₂H or (C₄H)(CH₂)₁₋₆(CHNH₂)CO₂H.

In one embodiment of the method,
R₉ is (C₆H₅)(CH₂)(CHNHBOC)CO₂H or (C₆H₅)(CH₂)(CHNH₂)CO₂H.

In one embodiment of the method,
R₃ is O(CH₂)₁₋₆R₉ or O(CH₂)R₉,
   where R₉ is phenyl.

In one embodiment of the method,
R₃ is OH and R₄ is

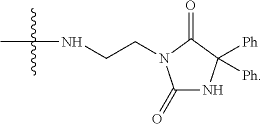 [SMILES: *NCCN1C(=O)NC(c2ccccc2)(c2ccccc2)C1=O]

In one embodiment of the method,
R₄ is

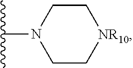

wherein R₁₀ is alkyl or hydroxyalkyl.

In one embodiment of the method, R₁₁ is —CH₂CH₂OH or —CH₃.

In one embodiment of the method, the compound has the structure:

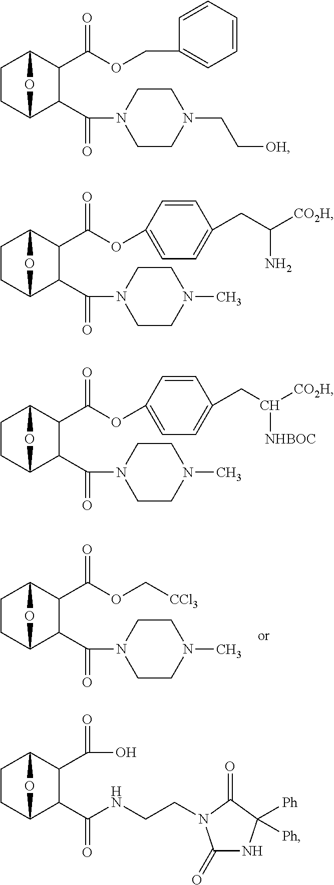

or a salt, zwitterion, or ester thereof.

In one embodiment of the method, the compound has the structure:

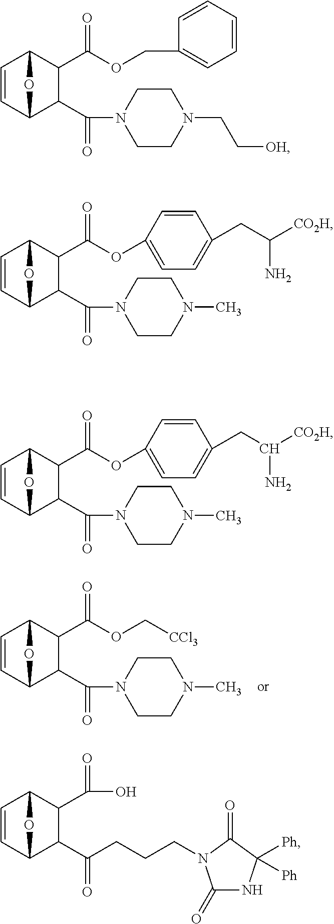

or a salt, zwitterion, or ester thereof.

In some embodiments, the compound having the structure:

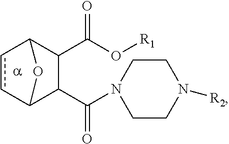

wherein
bond α is absent or present;
R₁ is C₂-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a salt, zwitterion, or eater thereof.

In some embodiments, the compound has the structure:

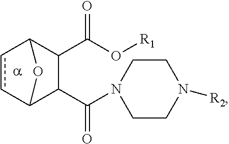

wherein
bond α is absent or present;
R₁ is C₃-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a salt, zwitterion, or ester thereof.

In some embodiments, the compound has the structure:

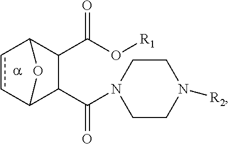

wherein
bond α is absent or present;
R₁ is C₄-C₂₀ alkyl, C₂-C₂₀ alkenyl, or C₂-C₂₀ alkynyl;
R₂ is H, C₁-C₁₂ alkyl, C₁-C₁₂ alkenyl, C₁-C₁₂ alkynyl, C₁-C₁₂ alkyl-(phenyl), C₁-C₁₂ alkyl-(OH), or C(O)C(CH₃)₃,
   or a salt, zwitterion, or ester thereof.

In some embodiments, the above compound having the structure:

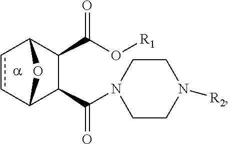

or a salt, zwitterion, or ester thereof.

In some embodiments, the above compound wherein
R₁ is —CH₂CH₃,
—CH₂CH₂CH₃,
—CH₂CH₂CH₂CH₃,
—CH₂CH₂CH₂CH₂CH₃,
—CH₂CH₂CH₂CH₂CH₂CH₃,
—CH₂CH₂CH₂CH₂CH₂CH₂CH₃,
—CH₂CH₂CH₂CH₂CH₂CH₂CH₂CH₂CH₃,
—CH₂CH₂CH₂CH₂CH₂CH₂CH₂CH₂CH₃, or
—CH₂CH₂CH₂CH₂CH₂CH₂CH₂CH₂CH₂CH₃.

In some embodiments, the above PP2A inhibitor wherein R, is —CH₂CH₂CH₂CH₂CH₂CH₂CH₂CH₂CH₂CH₂CH₂CH₂CH₂CH₂CH₂CH₂CH₃, or —CH₂CH₂CH₂CH₂CH₂CH₂CH₂CH₂CH₂CH₂CH₂CH₂CH₂CH₂CH₂CH₂CH₂CH₂CH₃.

In some embodiments, the above compound wherein
R₂ is —H, —CH₃, —CH₂CH₃, —CH₂-phenyl, —CH₂CH₂—OH, or —C(O)C(CH₃)₃.

In some embodiments, the compound having the structure:

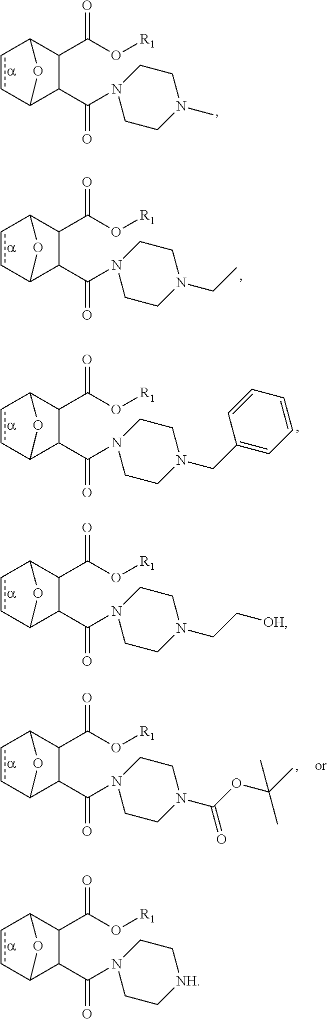

In some embodiments, the above compound wherein α is absent.

In some embodiments, the above compound wherein α is present.

In some embodiments, the compound having the structure:

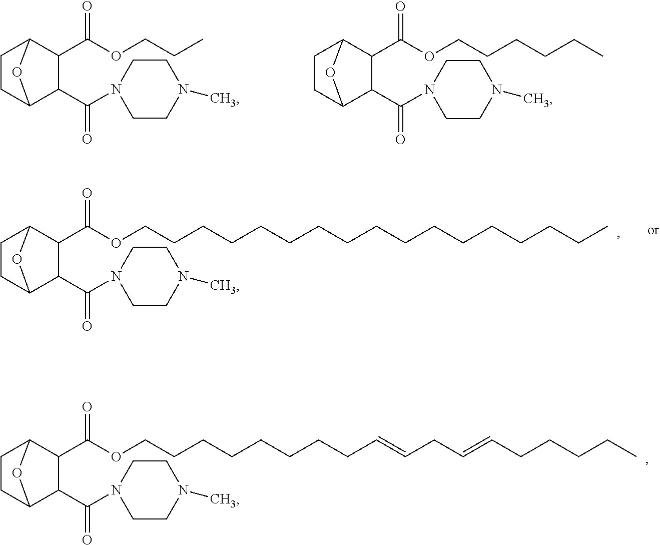 [SMILES: CCCOC(=O)C1C2CCC(O2)C1C(=O)N1CCN(C)CC1 ; CCCCCCOC(=O)C1C2CCC(O2)C1C(=O)N1CCN(C)CC1 ; CCCCCCCCCCCCCCCCCOC(=O)C1C2CCC(O2)C1C(=O)N1CCN(C)CC1 ; CCCCC/C=C/C/C=C/CCCCCCCCOC(=O)C1C2CCC(O2)C1C(=O)N1CCN(C)CC1]

or a salt, zwitterion, or ester thereof.

The present invention provides an amount of from 0.1 mg/m² to 5 mg/m² of any of the compounds disclosed herein for use in inhibiting protein phosphatase 2A (PP2A) in a subject.

The present invention provides an amount of from 0.1 mg/m² to 5 mg/m² of LB100 for use in inhibiting protein phosphatase 2A (PP2A) in a subject.

As used herein, “Type 2 Diabetes” is a disease consisting of an array of dysfunctions including, but not limited to, high blood glucose levels, insulin resistance, inadequate insulin secretion, and excessive or inappropriate glucagon secretion. Type 2 diabetes is associated with an array of complications, including microvascular, macrovascular, and neuropathic complications. Microvascular complications of diabetes include retinal, renal, and possibly neuropathic disease. Macrovascular complications include coronary artery and peripheral vascular disease. Diabetic neuropathy affects autonomic and peripheral nerves. Type 2 Diabetes is also associated with atherosclerosis, low glucose tolerance, dyslipidemia, hyperlipidemia, hypertriglyceridemia, and hypercholesterolemia.

As used herein, “Insulin Resistance” is a physiological condition where the natural hormone insulin becomes less effective at lowering blood sugar levels.

As used herein, “Insulin Sensitivity” is a measure of the tissue response to insulin and refers to insulin's ability to cause tissues to absorb glucose from the blood. A loss of insulin sensitivity may also be called insulin resistance.

As used herein, “reperfusion injury” is tissue damage, tissue death, cell damage, cell death, vascular leakage or endothelial dysfunction caused when blood supply returns to tissue, cells or blood vessels after a period of ischemia or lack of oxygen.

As used herein, “myocardial infarction” (MI), also known as a heart attack, is an infarction of the heart, causing cardiac tissue damage.

This is most commonly due to occlusion (blockage) of a coronary artery following the rupture of a vulnerable atherosclerotic plaque, which is an unstable collection of lipids (fatty acids) and white blood cells (especially macrophages) in the wall of an artery. The resulting ischemia (restriction in blood supply) and oxygen shortage, if left untreated for a sufficient period of time, can cause damage or death of heart muscle tissue (myocardium) due to reperfusion injury.

Examples of conditions caused by ischemia and that result in reperfusion injury include, but are not limited to, myocardial infarction; cerebral infarction (stroke) due to a disturbance in the blood vessels supplying blood to the brain; pulmonary infarction or lung infarction; Splenic infarction occurs when the splenic artery or one of its branches are occluded, for example by a blood clot; Limb infarction caused by arterial embolisms; skeletal muscle infarction caused by diabetes mellitus; bone infarction; testicle infarction; and sepsis.

As used herein, “disease characterized by a loss of protein function” is any disease wherein loss of protein function is a factor in the cause and/or progression of the disease.

As used herein, a “loss of protein function disease” or a “loss of function disease” is a “disease characterized by a loss of protein function” as defined above.

This invention is directed to loss of function diseases in which the treatment stabilizes a mutant protein and increases function.

In some embodiments, the compound is administered intravenously.

The present invention provides a pharmaceutical composition comprising LB-100 and at least one pharmaceutically acceptable carrier for use in treating cancer, a reperfusion injury, a disease characterized by loss of protein function, type-2 diabetes or a neurodegenerative disease.

The present invention also provides a package comprising:

1) a first pharmaceutical composition comprising an amount of a chemotherapeutic agent and a pharmaceutically acceptable carrier;
2) a second pharmaceutical composition comprising an amount of LB100 and a pharmaceutically acceptable carrier; and
3) instructions for use of the first and second pharmaceutical compositions together to treat cancer.

The present invention provides a pharmaceutical composition comprising LB-100 and a chemotherapeutic agent, and at least one pharmaceutically acceptable carrier for use in treating cancer.

In some embodiments, the pharmaceutical composition wherein the pharmaceutically acceptable carrier comprises a liposome.

In some embodiments, the pharmaceutical composition wherein the compound is contained in a liposome or microsphere, or the compound and the chemotherapeutic agent are contained in a liposome or microsphere.

The present invention provides a pharmaceutical composition comprising an amount of LB-100 for use in treating cancer simultaneously, contemporaneously or concomitantly with a chemotherapeutic agent.

In some embodiments, LB-100 for use as an add-on therapy or in combination with a chemotherapeutic agent for use in treating a subject afflicted with cancer.

In some embodiments, LB-100 in combination with a chemotherapeutic agent for use in treating cancer.

In some embodiments, a product containing an amount of LB 100 and an amount of a chemotherapeutic agent for simultaneous, separate or sequential use in treating a subject afflicted with cancer.

LB-100 has the structure:

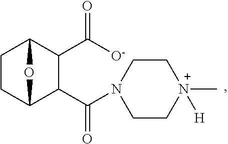 [SMILES: C[NH+]1CCN(C(=O)C2C(C(=O)[O-])[C@H]3CC[C@@H]2O3)CC1]

which may also be represented by the structure:

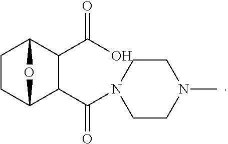 [SMILES: CN1CCN(C(=O)C2C(C(=O)O)[C@H]3CC[C@@H]2O3)CC1]

In some embodiments, the cancer is advanced and/or cannot be treated with surgery or radiation therapy.

In some embodiments, the non-small cell lung cancer is locally advanced, advanced, or has metastasized (has spread to other parts of the body) and cannot be treated with surgery.

In some embodiments, the ovarian cancer is ovarian cancer that is advanced or has metastasized in patients whose disease has not gotten better with other types of treatment or chemotherapy.

In some embodiments, the squamous cell carcinoma is of the head and neck that is locally advanced and cannot be treated with surgery.

In some embodiments, the testicular cancer is in patients who have already had surgery or radiation therapy.

In some embodiments of any of the above cancer, the cancer is in patients who have already had surgery or radiation therapy.

For the foregoing embodiments, each embodiment disclosed herein is contemplated as being applicable to each of the other disclosed embodiments. Thus, all combinations of the various elements described herein are within the scope of the invention.

The compound used in the method of the present invention is a protein phosphatase 2A (PP2A) inhibitor. Methods of preparation may be found in Lu et al., 2009; U.S. Pat. No. 7,998,957 B2; and U.S. Pat. No. 8,426,444 B2. Compound LB-100 is an inhibitor of PP2A in vitro in human cancer cells and in xenografts of human tumor cells in mice when given parenterally in mice. LB-100 inhibits the growth of cancer cells in mouse model systems.

In some embodiments, the anti-cancer agent is sorafenib, which is approved for the treatment of hepatocellular carcinoma (Llovet, J. M. et al. 2008; Kim, H. Y. et al. 2011).

In some embodiments, the anti-cancer agent is cisplatin, which is approved for the treatment of Bladder cancer (that cannot be treated with surgery or radiation therapy), Cervical cancer (that is advanced and cannot be treated with surgery or radiation therapy), Malignant mesothelioma (that cannot be treated with surgery), Non-small cell lung cancer (that is locally advanced, advanced, or has metastasized (has spread to other parts of the body) and cannot be treated with surgery), Ovarian cancer (that is advanced or has metastasized in patients whose disease has not gotten better with other types of treatment or chemotherapy), Squamous cell carcinoma (of the head and neck that is locally advanced and cannot be treated with surgery), and Testicular cancer (in patients who have already had surgery or radiation therapy).

As used herein, a “symptom” associated with disease or condition includes any clinical or laboratory manifestation associated with reperfusion injury and is not limited to what the subject can feel or observe.

As used herein, “treatment of the diseases” or “treating”, encompasses inducing prevention, inhibition, regression, or stasis of the disease or a symptom or condition associated with the disease.

As used herein, “inhibition” of disease progression or disease complication in a subject means preventing or reducing the disease progression and/or disease complication in the subject.

As used herein, “administering” an agent may be performed using any of the various methods or delivery systems well known to those skilled in the art. The administering can be performed, for example, orally, parenterally, intraperitoneally, intravenously, intraarterially, transdermally, sublingually, intramuscularly, rectally, transbuccally, intranasally, liposomally, via inhalation, vaginally, intraoccularly, via local delivery, subcutaneously, intraadiposally, intraarticularly, intrathecally, into a cerebral ventricle, intraventicularly, intratumorally, into cerebral parenchyma or intraparenchchymally.

The following delivery systems, which employ a number of routinely used pharmaceutical carriers, may be used but are only representative of the many possible systems envisioned for administering compositions in accordance with the invention.

Injectable drug delivery systems include solutions, suspensions, gels, microspheres and polymeric injectables, and can comprise excipients such as solubility-altering agents (e.g., ethanol, propylene glycol and sucrose) and polymers (e.g., polycaprylactones and PLGA's).

Other injectable drug delivery systems include solutions, suspensions, gels. Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating agents (e.g., starch polymers and cellulosic materials) and lubricating agents (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending agents (e.g., gums, zanthans, cellulosics and sugars), humectants (e.g., sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking agents, coating agents, and chelating agents (e.g., EDTA).

As used herein, “pharmaceutically acceptable carrier” refers to a carrier or excipient that is suitable for use with humans and/or animals without undue adverse side effects (such as toxicity, irritation, and allergic response) commensurate with a reasonable benefit/risk ratio. It can be a pharmaceutically acceptable solvent, suspending agent or vehicle, for delivering the instant compounds to the subject.

The compounds used in the method of the present invention may be in a salt form. As used herein, a “salt” is a salt of the instant compounds which has been modified by making acid or base salts of the compounds.

In the case of compounds used to treat an infection or disease, the salt is pharmaceutically acceptable. Examples of pharmaceutically acceptable salts include, but are not limited to, mineral or organic acid salts of basic residues such as amines; alkali or organic salts of acidic residues such as phenols. The salts can be made using an organic or inorganic acid. Such acid salts are chlorides, bromides, sulfates, nitrates, phosphates, sulfonates, formates, tartrates, maleates, malates, citrates, benzoates, salicylates, ascorbates, and the like. Phenolate salts are the alkaline earth metal salts, sodium, potassium or lithium. The term “pharmaceutically acceptable salt” in this respect, refers to the relatively non-toxic, inorganic and organic acid or base addition salts of compounds of the present invention. These salts can be prepared in situ during the final isolation and purification of the compounds of the invention, or by separately reacting a purified compound of the invention in its free base or free acid form with a suitable organic or inorganic acid or base, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, e.g., Berge et al. (1977) “Pharmaceutical Salts”, J. Pharm. Sci. 66:1-19).

The present invention includes esters or pharmaceutically acceptable esters of the compounds of the present method. The term “ester” includes, but is not limited to, a compound containing the R—CO—OR′ group. The “R—CO—O” portion may be derived from the parent compound of the present invention. The “R′” portion includes, but is not limited to, alkyl, alkenyl, alkynyl, heteroalkyl, aryl, and carboxy alkyl groups.

The present invention includes pharmaceutically acceptable prodrug esters of the compounds of the present method. Pharmaceutically acceptable prodrug esters of the compounds of the present invention are ester derivatives which are convertible by solvolysis or under physiological conditions to the free carboxylic acids of the parent compound. An example of a pro-drug is an alkyl ester which is cleaved in vivo to yield the compound of interest.

As used herein, an “amount” or “dose” of an agent measured in milligrams refers to the milligrams of agent present in a drug product, regardless of the form of the drug product.

The National Institutes of Health (NIH) provides a table of Equivalent Surface Area Dosage Conversion Factors below (Table A) which provides conversion factors that account for surface area to weight ratios between species.

TABLE A
Equivalent Surface Area Dosage Conversion Factors

| | To | | | | |
|---|---|---|---|---|---|
| From | Mouse 20 g | Rat 150 g | Monkey 3 kg | Dog 8 kg | Man 60 kg |
| Mouse | 1 | ½ | ¼ | ⅙ | 1/12 |
| Rat | 2 | 1 | ½ | ¼ | 1/7 |
| Monkey | 4 | 2 | 1 | ⅗ | ⅓ |
| Dog | 6 | 4 | 1⅔ | 1 | ½ |
| Man | 12 | 7 | 3 | 2 | 1 |

As used herein, the term “therapeutically effective amount” or “effective amount” refers to the quantity of a component that is sufficient to yield a desired therapeutic response without undue adverse side effects (such as toxicity, irritation, or allergic response) commensurate with a reasonable benefit/risk ratio when used in the manner of this invention. The specific effective amount will vary with such factors as the particular condition being treated, the physical condition of the patient, the type of mammal being treated, the duration of the treatment, the nature of concurrent therapy (if any), and the specific formulations employed and the structure of the compounds or its derivatives.

Where a range is given in the specification it is understood that the range includes all integers and 0.1 units within that range, and any sub-range thereof. For example, a range of 77 to 90% is a disclosure of 77, 78, 79, 80, and 81% etc.

As used herein, “about” with regard to a stated number encompasses a range of +one percent to −one percent of the stated value. By way of example, about 100 mg/m² therefore includes 99, 99.1, 99.2, 99.3, 99.4, 99.5, 99.6, 99.7, 99.8, 99.9, 100, 100.1, 100.2, 100.3, 100.4, 100.5, 100.6, 100.7, 100.8, 100.9 and 101 mg/m². Accordingly, about 100 mg/m² includes, in an embodiment, 100 mg/m².

It is understood that where a parameter range is provided, all integers within that range, and tenths thereof, are also provided by the invention. For example, “0.2-5 mg/m²” is a disclosure of 0.2 mg/m², 0.3 mg/m², 0.4 mg/m², 0.5 mg/m², 0.6 mg/m² etc. up to 5.0 mg/m².

All combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Abbreviations

AE Adverse Event
ALT/SGPT Alanine Aminotransferase/Serum Glutamic-Pyruvic Transaminase
AST/SGOT Aspartate Transaminase/Serum Glutamic-Oxaloacetic Transaminase
BID Bis in Die (twice a day)
BNP Brain Natriuretic Peptide
BUN Blood Urea Nitrogen
CFR Code of Federal Regulations
CR Complete Response
CRF Case Report Form
CTCAE Common Terminology Criteria for Adverse Events
DLT Dose Limiting Toxicity
ECG Electrocardiogram
ECOG Eastern Cooperative Oncology Group
FDA Food and Drug Administration
GCP Good Clinical Practice
GLP Good Laboratory Practice
HBV Hepatitis B Virus
HCV Hepatitis C Virus
HIV Human Immunodeficiency Virus
HNSTD Highest Non-Severely Toxic Dose
IRB/IEC Institutional Review Board/Independent Ethics Committee
ITT Intent-to-Treat
IV Intravenous
PD Progressive Disease
MAD Maximum Administered Dose
MHRA Medicine and Healthcare Products Regulatory Agency
MTD Maximum Tolerated Dose
NCI National Cancer Institute
NIH National Institutes of Health
NOAEL No Observed Adverse Effect Level
NOEL No Observed Effect Level
PBS Phosphate Buffered Saline
PK Pharmacokinetics
PR Partial Response
RD Recommended Dose
RECIST Response Evaluation Criteria in Solid Tumors
SAE Serious Adverse Event
SD Stable Disease
ULN Upper Limit of Normal
WBC White Blood Cell

### ECOG Performance Status

### Grade 0

Fully active, able to carry on all pre-disease performance without restriction.

### Grade 1

Restricted in physically strenuous activity but ambulatory and able to carry out work of a light or sedentary nature, e.g., light house work, office work.

### Grade 2

Ambulatory and capable of all self-care but unable to carry out any work activities. Up and about more than 50% of waking hours.

### Grade 3

Capable of only limited self-care, confined to bed or chair more than 50% of waking hours.

### Grade 4

Completely disabled. Cannot carry on any self-care. Totally confined to bed or chair.

### Grade 5

Dead.

### Cockcroft-Gault Formula

Creatinine clearance (CL_{cr}) in mL/min may be estimated using the following formula (for males and females):

$$Males \text{:}$$

$${CL}_{cr} = \frac{[ 140 - age ( yr ) ] \times [ weight ( kg ) ]}{72 \times serum creatinine ( mg / dL )}$$

$$Females \text{:}$$

$${CL}_{cr} = \frac{[ 140 - age ( yr ) ] \times [ weight ( kg ) ] \times 0.85}{72 \times serum creatinine ( mg / dL )}$$

### LB-100

LB-100 (3-(4methylpiperazine-carbonyl)-7-oxalobicyclo[2.2.1]heptane-2-carboxylic acid; NSC D753810) is a small molecule (MW 268), which inhibits protein phosphatase 2A (PP2A) about 80 fold more efficiently than protein phosphatase 1 (PP1). The compound has single agent activity in vitro and in vivo and potentiates the activity of cytotoxic agents including temozolomide, doxorubicin, docetaxel, and ionizing radiation in vivo. The mechanism of potentiation appears to be inhibition of cell cycle and mitotic checkpoints induced by non-specific DNA damaging agents, allowing dormant cancer cells to enter S phase and continue in mitosis despite acute DNA damage (Zhuang et al, 2009). LB-100 also appears to affect the vasculature inducing transient reversible vessel “leakiness” at high doses. Because of its unique mechanism of action and ability to enhance the activity of a broad spectrum of anti-cancer agents including ionizing radiation, LB-100 is useful for the treatment of many types of cancers

LB-100 is a water soluble enantiomeric zwitterion provided as a sterile solution for intravenous administration. As formulated in monosodium glutamate, pH 10.5, it is stable for months at −20° C. and for at least 8 hours at refrigerated temperatures. Phosphate buffered saline (PBS) was used as the vehicle for LB-100 administration in preclinical efficacy studies and sodium bicarbonate 4.2% for injection was used as the vehicle for GLP toxicity studies. Only the racemate has been studied as it has been shown that the separated enantiomers racemize rapidly in solution.

LB-100 has shown in vitro and in vivo activity as a single agent as well as potentiating the activity of cytotoxic agents including temozolomide, doxorubicin, docetaxel and ionizing radiation in vivo. LB-100 is active in combination with temozolomide or doxorubicin against xenografts of glioblastoma multiforme (GBM) and neuroblastoma (Lu et al 2009a, 2009b), pheochromocytoma (Martiniova et al, 2010), breast cancer (mouse and human, unpublished), fibrosarcoma (rat, Zhang at al, 2010), and melanoma. Racemic LB-100 used alone has modest single agent antitumor activity in vivo against diverse cell types of human cancer. Combined with temozolomide, doxorubicin or docetaxel, LB-100 potentiates their single agent activity, leading to regression of human cancer xenografts for periods of time greater than achieved with the standard cytotoxic drugs alone. Thus, the clinical potential of LB-100 lies in using it in combination with chemotherapy.

LB-100 has demonstrated in vivo activity both as a single agent and when combined with temozolomide, doxorubicin or docetaxel. LB-100 blocks several important DNA repair mechanisms rendering cancer cells more susceptible to damage by standard chemotherapeutic drugs. Normal cells appear to be less susceptible to this enhancing effect than cancer cells, potentially providing a greater therapeutic index for standard anti-cancer drugs.

### Toxicology

In a non-GLP dose ranging study in male Fischer rats conducted by the NCI, LB-100 was administered by daily intravenous (IV) infusion at 0.5, 0.75 and 1.5 mg/kg/day for 4 consecutive days. There were no unscheduled deaths in any of the treatment groups. A no-observed-adverse-effect-level (NOAEL) was not established in this study. The MTD was 0.75 mg/kg/day (about 4.5 mg/m²) when administered IV daily for 4 days. At 1.5 mg/kg/day, clinical observations included blood in urine (Day 4), lethargy (Days 3 and 4), and hind limb paresis (Day 4). At 1.5 mg/kg/day, adverse effects in kidney (nephrosis) in the distal convoluted tubules were seen in 3 of 3 rats; in the 0.75 mg/kg/day group, nephrosis was mild, and in the 0.5 mg/kg/day group, nephrosis was minimal. Primary clinical signs of blood in the urine and clinical chemistry findings of increased blood urea nitrogen and creatinine supported kidney and urinary bladder as target organs of toxicity. The transient hind limb paresis observed at the 1.5 mg/kg/day dose level had no histopathology correlates that would explain the paresis. Heart toxicity (epicardial hyperplasia with inflammation primarily on the epicardium of the atria) was observed in the 0.75 and 1.5 mg/kg/day groups. The hyperplasia was accompanied by subepicardial accumulation of mononuclear cells and eosinophils. One rat in the 1.5 mg/kg/day group had a large focus of inflammation with eosinophils associated with the aorta. Kidney, heart, femoral bone, liver and urinary bladder toxicity appeared to be dose-limiting toxicities in rats treated with LB-100 when administered IV once per day for 4 consecutive days.

In the GLP repeat-dose study in rats, LB-100 administered via daily intravenous (slow bolus) injection for 5 consecutive days to male and female Sprague Dawley rats at dose levels of 0.5, 0.75, and 1.25 mg/kg/day resulted in adverse, test article-related nephrosis of the kidneys in the 0.75 and 1.25 mg/kg/day group males and females, which persisted or progressed in the 0.75 and 1.25 mg/kg/day group males at the recovery necropsy. Test article-related effects on urinalysis parameters were observed in all treatment groups and included an increase in incidence and severity of urine occult blood in 0.5 mg/kg/day group males and 0.75 and 1.25 mg/kg/day group males and females, urine protein in 1.25 mg/kg/day females, and increase in microscopic observations of leukocytes in males and females of the 1.25 mg/kg/day group, and in one female in both the 0.5 and 0.75 mg/kg/day group on Day 5. These changes were reversible. LB-100 administration resulted in subacute subepicardial inflammation and/or mesothelial hypertrophy in the atria of males at ≧0.5 mg/kg/day and at 1.25 mg/kg/day in the females at the primary necropsy and was considered adverse in one 1.25 mg/kg/day group male. Minimal to mild subacute inflammation was observed in the epicardium and subepicardium of the left and/or right atrium of the heart in the 0.5, 0.75, and 1.25 mg/kg/day group males and the 1.25 mg/kg/day group females. One male in the 1.25 mg/kg/day group had mild subacute inflammation that was accompanied by minimal fibroplasia (plump fibroblasts) in the right atrium. Inflammation was often accompanied by mesothelial hypertrophy. There was a higher incidence of mesothelial hypertrophy in the 1.25 mg/kg/day group females when compared to the control group. Based on these findings, the severely toxic dose in 10% of the animals (STD 10) for this study was determined as 0.75 mg/kg/day. This dose corresponded to AUCₗₐₛₜ values of 596 and 691 ng·h/mL and C₀ values of 1804 and 2347 ng/mL for males and females, respectively, on study Day 4.

### Toxicity in Dogs

In a non-GLP dose ranging study, LB-100 administered intravenously (slow bolus push) to beagle dogs at dose levels of 0.1, 0.25, 0.5, and 1.0 mg/kg given every 4 days×4 doses (on study days 0, 4, 8, and 12) resulted in a no-observed-effect level (NOEL) of 0.25 mg/kg. There were no LB-100-related effects on survival. A possible test article-related clinical observation of intermittent tremors was noted in one female on study Day 13 following administration of LB-100 at 1.0 mg/kg. At dose levels of 0.5 and 1.0 mg/kg, lower body weight gains and food consumption were noted in females.

In the GLP repeat dose dog study, LB-100 was administered by intravenous injection (slow bolus push) at dose levels of 0.15, 0.30, and 0.75 mg/kg daily for 5 consecutive days. Test article-related lethality was observed in 2 of 10 animals in the 0.75 mg/kg/day group, a male and a female were found dead prior to administration of the fourth scheduled dose. The dosage level was reduced to 0.50 mg/kg/day for the 4^{th} and 5^{th} doses (study Days 3 and 4). Both animals dying after the 3^{rd} dose at 0.75 mg/kg/day had similar test article-related macroscopic and microscopic findings affecting the gastrointestinal tract, kidneys, injection sites (hemorrhage), spleen, larynx, lungs (including acute inflammation) and/or liver. Both animals had experienced emesis, decreased defecation, yellow and red mucoid feces, and red diarrhea; these changes were also observed in animals treated at the 0.3 mg/kg/day dose level.

Although the most noteworthy findings (mitotic figures and single cell necrosis of the renal tubular epithelial cells from the outer medulla and cortex) could be associated with altered renal function, these findings were not considered fatal lesions; therefore, the cause of death for each animal was considered undetermined but directly attributed to test article administration. Note: a dose of 0.75 mg/kg in the dog (average weight of 9 kg and BSA of 0.5 m²) is about 13.8 mg/m² or more than twice the MTD in the rat. This highest dose was selected because the dose range study in the dog revealed almost no signs of toxicity following a single dose of 1.0 mg/kg (approximately 18 mg/m²) in the dose ranging study. All other animals survived to the scheduled primary (study Day 5) and recovery (study Day 29) necropsies including the dogs receiving 0.75 mg/kg daily for 3 days and 0.5 mg/kg for doses 4 and 5. Test article-related histological changes at the Day 5 necropsy included erosion and focal hemorrhage within the gastrointestinal tract in the 0.75/0.5 mg/kg/day dose group. Single cell necrosis was observed throughout the gastrointestinal tract. These changes were reported as resolved in the recovery period. There were no ophthalmic findings or changes in electrocardiography parameters and blood pressures associated with test article administration in any treatment group.

During the recovery period, all surviving animals had body weight gains indicative of recovery, and the majority of the other observed clinical signs resolved within the first few days of the recovery period. At the primary necropsy (Day 5), test article-related macroscopic findings consisted of dark red discoloration of the kidneys, small spleens, and red discoloration (reddened mucosa or dark red areas) of various segments of the gastrointestinal tract in the 0.75/0.50 mg/kg/day group males and females. At the recovery necropsy (Day 29), no test article-related macroscopic finding were observed. The primary cause of small spleen size appeared to be due to less blood in the red pulp. Mild or moderate single cell (lymphoid) necrosis was seen in spleens microscopically. Test article-related effects on hematology and coagulation parameters at the Day 5 evaluation included higher red blood cell mass (red blood cell count, hemoglobin, and hematocrit), lower platelet counts, and prolonged activated partial thromboplastin time values in the animals of the 0.75/0.50 mg/kg/day group. In this group, lower platelet counts were statistically significantly lower only in the males, with the group mean level lower than the historical control group mean level. Lower platelet count in a female was not statistically significant but was considered test-article related. At the Day 29 evaluation, there were no residual effects of test article administration on hematology or coagulation parameters. Test article-related changes in urinalysis parameters observed at the Day 5 evaluation included lower specific gravity, higher urine volume, and increased presence of blood in the 0.75/0.50 mg/kg/day groups. At Day 29, no test article-related changes in urinalysis parameters were present.

Multilead (I, II, III, aVR, aVL, aVF, and V2) ECGs were recorded for all animals prior to randomization (Day −8) and for all surviving animals on Day 4 (recorded approximately 2 to 4 hours following dose administration) and Day 27. All the ECGs were qualitatively and quantitatively interpreted and within normal limits. No test article-related effects attributable to the test article administration were found at any dose level based on comparison of pretest and post-dosing group mean values and control values. No abnormalities in rhythm were found.

Blood pressure (systolic, diastolic, and mean arterial pressure) data were recorded for all animals once during the pretest period (Day −8) and for all surviving animals on study Day 4 (recorded approximately 2 to 4 hours following dose administration) and Day 27. Blood pressure was unaffected by test article administration. There were no statistically significant differences at the Days 4 and 27 evaluations when the control and test article-treated groups were compared.

In conclusion, administration of LB-100 via daily intravenous (slow bolus) injection for 5 consecutive days to male and female beagle dogs was well tolerated at the dosage level of 0.15 mg/kg/day. At dosage levels of 0.30 and 0.75/0.50 mg/kg/day, administration of LB-100 resulted in adverse clinical observations, lower body weights, and histological findings (congestion and nephrosis in kidneys, increased mitoses and single cell necrosis in liver, lymphoid depletion and single cell necrosis in thymus, and/or erosion and/or hemorrhage in stomach or intestines) correlating with effects on clinical pathology, organ weight, and/or macroscopic findings during the dosing period. Persistent adverse test article-related histological changes in the kidneys were observed in the 0.30 and 0.75/0.50 mg/kg/day group males and females at the Day 29 recovery necropsy. These changes were more indicative of a progression towards chronicity rather than recovery. In addition, lethality was observed at 0.75 mg/kg/day. Therefore, the Highest Non-Severely Toxic Dose (HNSTD) was 0.15 mg/kg, which corresponded to an AUCₗₐₛₜ for LB-100 of 267 and 335 ng·h/mL on study day 4 for males and females, respectively.

### Dose Rationale

In preclinical studies, schedules of daily LB-100 dose administration for 3, 4, or 5 days were studied. In the daily×5 day regimen, rat toxicity data indicated that some renal toxicity was evident following Day 4 administration of LB-100. It is suspected that this toxicity is accentuated by a fifth dose. Furthermore, LB-100 is expected to be administered to enhance activity in combination with a second agent.

Various schedules of LB-100 given once weekly or three times weekly, for example, potentiated the activity of several standard cytotoxic drugs. Thus, a daily×3 dose of LB-100 is believed to be sufficient for potentiation of cytotoxic drugs such as docetaxel and should be less renal-toxic than a daily×5 dose.

### Patient Population

Approximately 42 patients are enrolled in the study. Of these, approximately 18 patients are enrolled in Part 1 (single agent LB-100), followed by approximately 18 patients in Part 2 (LB-100 in combination with docetaxel). An additional 6 patients (3 in each Part) are enrolled in the MTD confirmation cohorts.

### Trial Objectives and Purpose

### Primary Objectives

### Part 1/Single Agent LB-100

To determine the safety and tolerability of single agent LB-100 administered intravenously to patients with advanced solid tumors
To determine the maximum tolerated dose (MTD) of single agent LB-100 in this population

### Part 2/Combination Therapy LB-100 and Docetaxel

To determine the safety and tolerability of a combination therapy of intravenous LB-100 and docetaxel in patients with advanced solid tumors
To determine the maximum tolerated dose (MTD) and recommended Phase II dose (RD) of LB-100 when administered in combination with docetaxel in this population

### Secondary Objectives

### Part 1/Single Agent L-100

To determine tumor response of single agent LB-100 in patients with advanced solid tumors
To determine the pharmacokinetics (PK) of single agent LB-100

### Part 2/Combination Therapy LB-100 and Docetaxel

To determine tumor response of a combination therapy of intravenous LB-100 and docetaxel in patients with advanced solid tumors
To determine the PK of LB-100 when given in combination with docetaxel

### Overview of Trial Design

This is a Phase I, open label, multicenter, two-part, dose-escalation study of LB-100 administered intravenously in patients with advanced solid tumors. Two dose regimens are explored. In the first regimen (Part 1), single agent LB-100 is given daily on Days 1 through 3 of every 21 day cycle. In the second regimen (Part 2), docetaxel-naïve patients are treated with LB-100 given daily on Days 1 through 3 in combination with a fixed dose of docetaxel (either 60 mg/m² or 75 mg/m²) given on Day 2 of every 21 day cycle. Once the MTD of single agent LB-100 is reached, 3 additional patients are enrolled in an MTD confirmation cohort. Part 2 enrolls patients once the MTD of single agent LB-100 is determined in Part 1.

The starting dose of L8-100 for Part 1 is 0.25 mg/m². The starting dose of LB-100 for Part 2 is 2 dose levels below the MTD determined in Part 1, administered in combination with docetaxel 60 mg/m². Once the MTD of LB-100+docetaxel 60 mg/m² is determined, the dose of LB-100 is reduced 2 dose levels and administered in combination with docetaxel 75 mg/m². The dose of LB-100 is then be escalated to the full LB-100 dose (of the LB-100+docetaxel 60 mg/m² MTD) and administered in combination with docetaxel 75 mg/m². Once the MTD of the combination of LB-100+docetaxel (either 60 mg/m² or 75 mg/m²) is reached, 3 additional patients are enrolled in an MTD/RD confirmation cohort. The overall study design is presented in Scheme 1.

LB-100 is administered in escalating dose levels. Enrollment in each dose cohort proceeds in the standard “3+3” schema. In each cohort, the first patient enrolled completes Cycle 1 treatment and safety monitoring prior to the enrollment of the second and third patients; the second and third patients may be enrolled simultaneously. DLTs are evaluated and recorded during the first 2 cycles of treatment.

Initially, the decision to escalate to the next dose level is be based on the observation of DLT during Cycle 1. If no DLT is observed, escalation proceeds to the next cohort. If a DLT is observed in Cycle 2 in the study, the escalation strategy is evaluated moving forward. Escalation continues until the maximum administered dose (MAD), maximum tolerated dose (MTD) and recommended dose (RD) are determined.

In both regimens, once the MTD is determined, intra-patient dose escalation is allowed at the discretion of the Investigator, to patients currently receiving a lower dose of LB-100 for two or more cycles with no unacceptable toxicities or disease progression.

### End of Study

The end of the study is defined as the date of the last visit of the last patient undergoing treatment.

### Drug Product

Each vial of LB-100 sterile injection contains 10 mL of a 1.0 mg/mL solution of LB-100 in monosodium glutamate, pH 10.5. The appropriate dose is to be taken from the vial and added to 50 mL of normal saline (0.9%) and infused over 15 minutes. Dilution in saline is done to reduce the pH so that the infusate is non-irritating, but extravasation is to be avoided.

### Duration of Therapy

Patients may receive up to 6 cycles of study therapy, unless unacceptable toxicity, disease progression or intercurrent illness requires discontinuation. Patients may continue treatment beyond 6 cycles if the Investigator determines that additional treatment would provide further benefit for the patient as long as toxicity remains acceptable.

### Trial Discontinuation

For reasonable cause, either the Investigator or the Sponsor may terminate this study prematurely. Written notification of the termination is required. Conditions that may warrant termination include, but are not limited to:
The discovery of an unexpected, significant, or unacceptable risk to the patients enrolled in the study.
Failure of the Investigator to enter patients at an acceptable rate.
Insufficient adherence to protocol requirements (non-compliance).
Lack of evaluable and/or complete data.
Decision to modify the developmental plan of the drug.
A decision on the part of the Sponsor to suspend or discontinue development of the drug.

In the case that the trial is discontinued due to reasons other than unforeseen risk, patients who are currently receiving drug and are deriving benefit from the treatment may be allowed to continue receiving treatment.

### Selection and Withdrawal of Subjects

### Inclusion Criteria:

1. Part 1 only: Patients with histologically or cytologically proven progressive or metastatic solid tumors who have failed standard treatment and have no other effective treatment available.
   Part 2 only: Patients with histologically or cytologically proven progressive or metastatic solid tumors who have failed standard treatment and have no other effective treatment available, or docetaxel-naïve patients who have failed standard treatment and have tumors for which a docetaxel-based regimen would be appropriate.
2. Part 2 only: Patients must be docetaxel-naïve.
3. Patients must have a life expectancy of at least 12 weeks.
4. Patients must have an ECOG performance status of 0 or 1 (see Appendix I).
5. Patients must be men and women ≧18 years of age.
6. Patients must have recovered from all acute adverse effects (excluding alopecia) of prior therapies to baseline or 5 grade 1 prior to study entry.
7. Patients must have adequate bone marrow function, defined as an absolute neutrophil count ≧1.5×10⁹/L and a platelet count ≧100×10⁹/L.
8. Patients must have adequate renal function, defined as serum creatinine ≦1.2 mg/dL (if >1.2 mg/dL, a calculated creatinine clearance [Cockcroft-Gault method] must be ≧60 mL/min/1.73 m²; see Appendix II).
9. Patients must have adequate hepatic function, defined as:
   Part 1 only: plasma total bilirubin ≦1.5 mg/dL, alanine transaminase (ALT) and aspartate transaminase (AST)≦2.5×ULN (upper limit of normal).
   Part 2 only: plasma total bilirubin ≦ULN; ALT and/or AST≦1.5×ULN concomitant with alkaline phosphatase ≦2.5×ULN.
10. Female patients of childbearing potential must have a negative serum or urine pregnancy test result at time of pre-treatment screening.
11. Patients with reproductive potential must agree to use at least one form of barrier contraception prior to study entry and for up to 30 days beyond the last administration of study drug.
12. Patients must be capable of providing informed consent and must be willing to provide written informed consent prior to the start of any study-specific procedures.

### Exclusion Criteria:

1. Patients may not have had prior chemotherapy, radiotherapy, hormonal therapy, or biologic therapy in the 4 weeks prior to study entry with the exception of mitomycin C or nitrosoureas, for which patients must be 6 weeks from prior treatment. For patients who have been treated with targeted therapy, 5 half-lives of that therapy (or 28 days, whichever is shorter) must have passed prior to enrollment in the study.
2. Part 2 only: Patients may not have had prior treatment with docetaxel.
3. Part 2 only: Patients with plasma total bilirubin >ULN; ALT and/or AST>1.5×ULN concomitant with alkaline phosphatase >2.5×ULN.
4. Patients may not have any concomitant condition that could compromise the objectives of this study and the patients' compliance and ability to tolerate this therapy and complete at least 2 cycles of therapy, including, but not limited to the following:
   Congestive heart failure or uncontrolled angina pectoris, previous history of myocardial infarction within 1 year from study entry, uncontrolled hypertension, or dysrhythmias.
   Active infection.
   Unstable diabetes mellitus.
   Psychiatric disorder that may interfere with consent and/or protocol compliance.
   Uncontrolled seizure activity.
   Prior history of inflammatory bowel disease.
   Prior history of pulmonary fibrosis.
   Prior history of cardiomyopathy.
5. Patients with known brain metastases (due to their poor prognosis and because they often develop progressive neurologic dysfunction that would confound the evaluation of neurologic and other adverse events).
6. Pregnant or breastfeeding women.
7. Patients with another malignancy in the past 3 years except: curatively treated non-melanoma skin cancer, or carcinoma in situ (either cervix or breast) that does not require further treatment.
8. Patients with known active HIV, HBV, or HCV infection.
9. Part 2 only: Patients with a history of severe hypersensitivity reaction to drugs formulated with polysorbate 80 (for example, drugs formulated with polysorbate 80 include, but are not limited to: Aranesp®, Eprex®, Cordarone®, some vaccines).
10. Part 2 only: Patients with ≧grade 2 peripheral neuropathy.
11. Patients with an underlying diagnosis or disease state associated with an increased risk of bleeding.

### Withdrawal Criteria:

Protocol therapy will be discontinued and patients withdrawn from the study for any of the following reasons:
Progressive disease.
Development of DLT through the end of Cycle 2.
Development of toxicity which, in the Investigator's judgment, precludes further treatment.
Patient refusal.
Lost to follow-up/noncompliance.
Intercurrent illness.
At the discretion of the Investigator (e.g., rapid clinical deterioration).
Pregnancy.
Study termination.

### Treatment of Subjects: Drug Preparation and Administration

### LB-100:

LB-100 was supplied as a sterile solution for intravenous administration. LB-100 is to be stored at −20° C. (allowable range: −25° C. to −10° C.). Each vial contains LB-100 at a concentration of 1 mg/mL.

The proper dose is drawn up in a sterile syringe and added to 50 mL of normal saline (0.9%). Following dilution in normal saline, LB-100 should be administered within 8 hours.

LB-100 Administration, Part 1 and Part 2:

On Days 1, 2 and 3 of each cycle, LB-100 was infused over 15 minutes.

A revised protocol comprises adding LB100 to 500 mL of normal saline (0.9%) and infusing over two hours instead of placing the LB-100 in 50 ml of saline and infusing over 15 minutes.

### Docetaxel:

Docetaxel is commercially available in a two-vial formulation (injection concentrate and diluent) in two strengths (Taxotere® 80 mg/2 mL and Taxotere® 20 mg/0.5 mL); see package insert (Appendix III) for preparation instructions. The appropriate dose is added to either 0.9% sodium chloride solution or 5% dextrose solution and administered intravenously as a 1-hour infusion. Following dilution, docetaxel should be used within 4 hours, including the 1-hour intravenous administration.

Premedication for docetaxel: Patients should be premedicated with oral corticosteroids [e.g., dexamethasone 16 mg per day (8 mg BID)] for 3 days starting 1 day prior to docetaxel administration.

### Administration Sequence, Part 2 Only:

On Days 1, 2 and 3 of each cycle, patients take the first of 2 daily doses of oral corticosteroids prior to study drug administration and the second dose of oral corticosteroid approximately 12 hours after the first dose.
On Days 1, 2 and 3 of each cycle, LB-100 are infused over 15 minutes.
On Day 2 of each cycle, 1 hour after the end of the LB-100 injection, the 1-hour docetaxel infusion is administered.

### Dose Escalation Scheme

### Part 1—Single Agent LB-100

LB-100 was administered daily on Days 1 through 3 of every 21 day cycle to patients at the following dose levels:

TABLE 1
Dose Levels for Treatment Part 1: LB-100 Single Agent

| Dose Level | LB-100 (mg/m²) |
|---|---|
| −1^{(a)} | 0.12 |
| 1 (Starting | 0.25 |
| dose) | |
| 2 | 0.50 |
| 3 | 0.83 |
| 4 | 1.25 |
| 5 | 1.75 |
| 6 | 2.33 |
| 7+ | increments not to |
| | exceed 33% of |
| | previous dose |

^{(a)}In the event that DLT is observed at Dose Level 1, subsequent patients will be enrolled in Dose Level −1.

LB-100 dose levels will increase as shown in Table 1. Enrollment in each dose cohort will proceed in the standard “3+3” schema where 3 patients are initially enrolled per cohort; Scheme 2. In each cohort, the first patient enrolled will complete Cycle 1 treatment and safety monitoring prior to the enrollment of the second and third patients; the second and third patients may be enrolled simultaneously. Dose limiting toxicities (DLTs) will be evaluated and recorded during the first 2 cycles of treatment. Initially, the decision to escalate to the next dose level will be based on the observation of DLT during Cycle 1. If no DLT is observed, escalation will proceed to the next cohort. Note: if a DLT is observed in Cycle 2 in the study, the escalation strategy will be evaluated moving forward. If, however, DLT is observed in 1 of the 3 patients, an additional 3 patients must be enrolled at that dose. In a cohort of 6, if one patient has DLT, then dose escalation may proceed; if 2 or more patients have DLT, then that dose will be considered the maximum administered dose (MAD) and dose escalation will cease. The MTD is defined as one dose level below the MAD. Once the MTD of single agent LB-100 is reached, 3 additional patients will be enrolled in an MTD confirmation cohort. At least 6 patients must be treated at the MTD to determine the safety of that dose as a basis for the starting dose of LB-100 in Part 2 of this study.

### Part 2—Combination Therapy LB-100 and Docetaxel

Once the MTD of single agent LB-100 is determined (Part 1), enrollment in Part 2 is initiated. In Part 2, docetaxel-naïve patients are treated with LB-100 given daily on Days 1 through 3 in combination with a fixed dose of docetaxel (either 60 mg/m² or 75 mg/m²) given on Day 2 of every 21 day cycle. Patients should be premedicated with oral corticosteroids starting 1 day prior to docetaxel administration, see Section 0. The starting dose of LB-100 is 2 dose levels below the MTD determined in Part 1. The fixed dose of docetaxel is initially set at 60 mg/m².

LB-100+Docetaxel (60 mg/m²)

LB-100 is administered in escalating dose levels. Cohorts of 3 patients are treated with the combination therapy of LB-100 and docetaxel (60 mg/m²); see Scheme 2. In each cohort, the first patient enrolled completes Cycle 1 treatment and safety monitoring prior to the enrollment of the second and third patients; the second and third patients may be enrolled simultaneously. Dose escalation proceeds as described above for Part 1 until the MTD of the LB-100+docetaxel 60 mg/m² combination is determined.

LB-100+Docetaxel (75 mg/m²)

After determination of the MTD of the LB-100+docetaxel 60 mg/m² combination, patients are treated with LB-100+docetaxel 75 mg/m². LB-100 is initially be administered to a cohort of 3 patients at 2 dose levels below the LB-100 dose of the LB-100+docetaxel 60 mg/m² MTD. The first patient enrolled completes Cycle 1 treatment and safety monitoring prior to the enrollment of the second and third patients; the second and third patients may be enrolled simultaneously.
If 0 of the 3 patients have DLT, the dose of LB-100 is escalated for the next cohort of 3 patients toward the full LB-100 dose (of the LB-100+docetaxel 60 mg/m² MTD), administered in combination with docetaxel 75 mg/m².
If 1 of the 3 patients has DLT, then an additional 3 patients (total of 6 patients) are enrolled to the cohort; if only ⅙ patients has DLT, then the dose of LB-100 is escalated for the next cohort of 3 patients toward the full LB-100 dose in combination with docetaxel 75 mg/m².
If more than 1 of 6 patients has DLT, the dose of LB-100 is not escalated.

The dose of LB-100 will not be escalated beyond the LB-100 dose of the LB-100+docetaxel 60 mg/m² MTD. Once the MTD of the combination of LB-100+docetaxel (either 60 mg/m² or 75 mg/m²) is reached, 3 additional patients will be enrolled in an MTD/RD confirmation cohort. At least 6 patients must be treated at the MTD/RD to determine the appropriateness of those doses for further study.

### Dose-Limiting Toxicity

The NCI Common Terminology Criteria for Adverse Events (CTCAE) Version 4.0 will be used to grade toxicity. DLT is defined as any of the following adverse events occurring through the end of Cycle 2 of treatment and considered to be possibly, probably, or definitely related to study treatment:
Nausea/vomiting of Grade 3 or greater despite maximal antiemetic therapy.
Diarrhea of Grade 3 or greater despite maximal antidiarrheal therapy.
Any other Grade 3 or 4 non-hematologic toxicity.
Grade 4 neutropenia observed for greater than 5 days duration or Grade 3 neutropenia associated with fever of any duration or where sepsis results.
Grade 4 thrombocytopenia.

### Maximum Administered Dose (MAD)

If 2 or more patients have DLT at a dose level, it will be considered the MAD and dose escalation will cease.

### Maximum Tolerated Dose-Recommended Dose (MTD/RD)

The MTD is defined as the dose level below the MAD at which no more than one out of 6 evaluable patients experiences DLT. The RD is the dose recommended for Phase II study.

### Dose Modifications: Dose Reduction/Treatment Delay

### LB-100 Modifications

### Part 1 and Part 2:

If a patient experiences an adverse event that meets DLT criteria (see Section 0), then the patient will be taken off study and receive no further treatment, unless the Investigator feels the patient will benefit from continuing therapy.

Patients who experience grade 2 toxicity (i.e., a non-DLT event which is considered to be possibly, probably or definitely related to the investigational agent) which resolves to ≦grade 1 or to baseline levels in 14 days or less can be retreated with a dose reduction, with the next cycle given at the subsequent lower dose level of LB-100; otherwise, they should be taken off study.

In the event that a patient must have treatment interrupted because the Investigator judges that the patient's current condition would be exacerbated by continuing treatment, then the next cycle will be given at the subsequent lower dose level of LB-100.

### Cycle 1 Doses:

If the Day 2 or Day 3 dose of LB-100 cannot be administered, then the patient should be taken off study.

### Cycle 2 Doses:

If the Day 2 dose of LB-100 cannot be administered, then the patient should be taken off study. If the Day 3 dose of LB-100 cannot be administered, then the dose will be skipped and administration of the investigational agent will resume in Cycle 3 as long as the criteria above are met.

### Cycle 3 and Later Doses:

If the Day 2 or Day 3 dose of LB-100 cannot be administered, then the dose will be skipped and administration of the investigational agent will resume in the subsequent cycle as long as the criteria above are met.

If more than one dose reduction of LB-100 is required, the patient will be removed from the study. Any patient experiencing a dose interruption or delay for treatment-related toxicity lasting longer than 14 days will be removed from the study.

### Docetaxel Modifications

Part 2 only:

Docetaxel administration should be delayed for the following docetaxel-related events:

Grade 2 or greater non-skin adverse event with the following exceptions:
Grade 2 fatigue or laboratory abnormalities do not require a treatment delay
Grade 3 skin adverse event

Grade 3 laboratory abnormality that is clinically significant with the following exceptions:
Grade 3 lymphopenia does not require dose delay
Docetaxel treatment should be held for the following:
   ANC<1500 cells/mm3
   Total bilirubin >ULN or if AST and/or ALT>1.5×ULN with alkaline phosphatase >2.5×ULN

Any adverse event, laboratory abnormality or intercurrent illness which in the judgment of the Investigator warrants delaying the dose Any grade 4 toxicity.

After Cycle 2, patients may receive growth factors at the discretion of the Investigator.

Docetaxel dose reduction is allowed as follows:
For patients treated with docetaxel 60 mg/m², the dose may be reduced to 50 mg/m² for subsequent cycles for docetaxel-related toxicity; no further reduction is allowed.
For patients treated with docetaxel 75 mg/m², the dose may be reduced to 60 mg/m² for subsequent cycles for docetaxel-related toxicity. If a second dose reduction is necessary, the dose may be reduced to 50 mg/m² for subsequent cycles; no further reduction is allowed.

### Intra-Patient Dose Escalation

Part 1 and Part 2: Once the MTD is determined, intra-patient dose escalation is allowed at the discretion of the Investigator, to patients currently receiving a lower dose of LB-100 for two or more cycles with no unacceptable toxicities or disease progression.

### Concomitant Treatment

Patients may not receive any other anticancer agent while on-study.
Patients may continue their baseline medication(s) as long as they are not prohibited (e.g., anticancer agents).
Palliative and supportive care (i.e., anti-emetics, bisphosphonates) for disease related symptoms will be offered to all patients in the study per institutional prescribing practices.
All concomitant medications that are currently in use or that become necessary during the study should be recorded in the CRF.

TABLE 2

| | Pre- | Cycle 1 and Cycle 2 (exceptions noted) | | | | | | Cycle 3 Subsequent Cycles | | | | | After | |
|---|---|---|---|---|---|---|---|---|---|---|---|---|---|---|
| | treat- | Day | Day | Day | Day | Day | Day | Prior to/ | Days | Day | Day | Day | every 2 | Off- |
| Evaluation | ment | 1 | 2 | 3 | 8^{e} | 15^{e} | 22^{f} | Day 1^{f} | 2 & 3 | 8^{e} | 15^{e} | 22^{f} | cycles | Study |
| Informed consent | X^{a} | | | | | | | | | | | | | |
| Medical history | X^{c} | | | | | | | | | | | | | |
| Physical exam | X^{c} | X | | | X | | | X | | | | | | X |
| Height | X^{c} | | | | | | | | | | | | | |
| Weight | X^{c} | X | | | X | X | X | X | | | | | | X |
| Vital signs^{g} | X^{c} | X | X | X | X | X | X | X | X | X | X | X | | X |
| ECG^{h} | X^{a} | X | | X | | | X | X | | | | | | X |
| Cardiac troponins | X^{a} | X | | | | | | X | | | | | | |
| & BNP | | | | | | | | | | | | | | |
| MUGA/Echo- | X^{a} | X | | | | | | X | | | | | | |
| cardiogram | | | | | | | | | | | | | | |
| ECOG performance | X^{c} | X | | | X | | | X | | | | | | X |
| status | | | | | | | | | | | | | | |
| Tumor measurementⁱ | X^{a} | | | | | | | | | | | | X | X |
| (tumor markers, | | | | | | | | | | | | | | |
| if applicable) | | | | | | | | | | | | | | |
| Hematology^{j} | X^{b} | X | | X | X | X | X | X | | X | X | X | | X |
| Blood chemistry^{k} | X^{b} | X | | X | X | X | X | X | | X | X | X | | X |
| Urinalysis | X^{b} | X | | X | X | X | X | X | | X | X | X | | X |
| Pregnancy test^{l} | X^{c} | | | | | | | | | | | | | |
| PK blood sampling | | X^{d} | | X^{d} | | | | | | | | | | |

| | | | |
|---|---|---|---|
| Concomitant | X^{c} | <------------------------------------- throughout study -------------------------------------> | X |
| medications | | | |
| Adverse events | | <------------------------------------- throughout study -------------------------------------> | X |

^{a}within 28 days prior to treatment.
^{b}within 14 days prior to treatment.
^{c}within 7 days prior to treatment.
^{d}Cycle 1 only, in selected patients only, see Section 8.3 for pharmacokinetic (PK) blood sampling collection times.
^{e}in case of scheduling conflicts on the specified Days 8 and 15, ±3 day windows can apply.
^{f}“Day 22” = Day 1 of next cycle for patients continuing treatment. Day 1 evaluations for Cycle 2 and subsequent cycles are to be done within 3 days prior to next cycle drug administration. These tests do not need to be repeated if done on Day 22 of prior cycle.
^{g}vital signs including blood pressure, heart rate, respiration rate, and temperature. Part 1, on Days 1-3: before LB-100 infusion, within 15 minutes after end of infusion, and at 2 hours after end of infusion. Part 2, on Days 1 and 3: before LB-100 infusion, within 15 minutes after end of infusion, and at 2 hours after end of infusion. Part 2, on Day 2: before LB-100 infusion and within 15 minutes after end of the LB-100 infusion; before the docetaxel infusion, within 15 minutes after the end of the docetaxel infusion and at 2 hours after the end of the docetaxel infusion.
^{h}ECG at screening and Cycle 1 only: on Cycle 1 Day 1 and Day 3 (before infusion, within 15 minutes after end of infusion, and at 2 hours after end of infusion) and on Cycle 1 Day 22 (i.e., Day 1 of Cycle 2 for patients who begin Cycle 2 treatment); prior to each treament cycle, at off-study and as clinically indicated.
Note:
just for patients providing PK blood samples on Days 1 and 3, an additional ECG will be done at 4 hours after the end of the LB-100 infusion.
ⁱtumor measurement by RECIST version 1.1 and tumor markers, if applicable: the same method used at baseline for a patient should be used consistently for all evaluations throughout the study.
^{j}hematology including hemoglobin, WBC with differential, and platelet count.
^{k}blood chemistry including sodium, potassium, BUN, glucose, SGOT/SGPT (ALT/AST), alkaline phosphatase, total protein, total bilirubin, albumin, creatinine, and calcium.
^{l}pregnancy test; for women of childbearing potential, a negative pregnancy test (urine or serum) must be documented.

The below evaluation protocols are described in Table 2.

### Pre-Treatment

### Within 28 Days Prior to Treatment:

Informed consent form signed
ECG
Cardiac troponins and BNP
MUGA or Echocardiogram; same method used at baseline for a patient should be used consistently throughout the study
Tumor measurements (RECIST version 1.1), tumor markers, if applicable; the same method used at baseline for a patient should be used consistently for all evaluations throughout the study

### Within 14 Days Prior to Treatment:

Hematology including hemoglobin, WBC with differential, and platelet count
Blood chemistry including sodium, potassium, BUN, glucose, SGOT/SGPT (ALT/AST), alkaline phosphatase, total protein, total bilirubin, albumin, creatinine, and calcium
Urinalysis

### Within 7 Days Prior to Treatment:

Physical exam
Medical history
Height
Weight
Vital signs including blood pressure, heart rate, respiration rate, and temperature
ECOG performance status
Concomitant medications
Pregnancy test; for women of childbearing potential, a negative pregnancy test (urine or serum) must be documented

### During Treatment

Adverse events and concomitant medications will be monitored and recorded throughout the study. To monitor for cardiac toxicity, ECG, cardiac troponins and BNP, and MUGA or echocardiogram assessments will be done as described in Sections 0 through 0, and as clinically indicated.

### Cycle 1

### Day 1

Tests done within 3 days prior to treatment do not need to be repeated.
Cardiac troponins and BNP
MUGA or Echocardiogram
Physical exam
Weight
ECOG performance status
Vital signs: before LB-100 infusion, within 15 minutes after the end of infusion, and at 2 hours after the end of the infusion.
ECG: before LB-100 infusion, within 15 minutes after the end of infusion, and at 2 hours after the end of the infusion. Note: just for patients providing PK blood samples, an additional ECG will be done at 4 hours after the end of the infusion.
Hematology
Blood chemistry
Urinalysis
Selected patients only: Blood samples collected for PK analysis; see Section 0 for patients and specific timepoints.

### Day 2

### During Part 1:

Vital signs: before LB-100 infusion, within 15 minutes after the end of infusion, and at 2 hours after the end of the infusion.

### During Part 2:

Vital signs: before LB-100 infusion and within 15 minutes after the end of the LB-100 infusion; before the docetaxel infusion, within 15 minutes after the end of the docetaxel infusion and at 2 hours after the end of the docetaxel infusion.

### Day 3

Vital signs: before LB-100 infusion, within 15 minutes after the end of infusion, and at 2 hours after the end of the infusion.
ECG: before LB-100 infusion, within 15 minutes after the end of infusion, and at 2 hours after the end of the infusion. Note: just for patients providing PK blood samples, an additional ECG will be done at 4 hours after the end of the infusion.
Hematology
Blood chemistry
Urinalysis
Selected patients only: Blood samples collected for PK analysis; see Section 0 for patients and specific timepoints.

### Days 8 and 15±3 Days

Physical exam (Day 8 only)
Weight
ECOG performance status (Day 8 only)
Vital signs
Hematology
Blood chemistry
Urinalysis

### Day 22 (Before Next Cycle)

Weight
Vital signs
Hematology
Blood chemistry
Urinalysis
ECG

### Cycle 2

Day 1 (within 3 Days Prior to Study Treatment)

Tests done on Day 22 of prior cycle do not need to be repeated.
Cardiac troponins and BNP
MUGA or Echocardiogram
ECG
Physical exam
Weight
ECOG performance status
Vital signs: before LB-100 infusion, within 15 minutes after the end of infusion, and at 2 hours after the end of the infusion.
Hematology
Blood chemistry
Urinalysis

### Day 2

### During Part 1:

Vital signs: before LB-100 infusion, within 15 minutes after the end of infusion, and at 2 hours after the end of the infusion.

### During Part 2:

Vital signs: before LB-100 infusion and within 15 minutes after the end of the LB-100 infusion; before the docetaxel infusion, within 15 minutes after the end of the docetaxel infusion and at 2 hours after the end of the docetaxel infusion.

### Day 3

Vital signs: before LB-100 infusion, within 15 minutes after the end of infusion, and at 2 hours after the end of the infusion.
Hematology
Blood chemistry
Urinalysis

### Days 8 and 15±3 Days

Physical exam (Day 8 only)
Weight
ECOG performance status (Day 8 only)
Vital signs
Hematology
Blood chemistry
Urinalysis

### Day 22 (Before Next Cycle)

Weight
Vital signs
Hematology
Blood chemistry
Urinalysis

### Cycle 3 and Subsequent Cycles

Day 1 (within 3 Days Prior to Study Treatment)

Tests done on Day 22 of prior cycle do not need to be repeated.
Cardiac troponins and BNP
MUGA or Echocardiogram
ECG
Physical exam
Weight
ECOG performance status
Vital signs: before LB-100 infusion, within 15 minutes after the end of infusion, and at 2 hours after the end of the infusion.
Hematology
Blood chemistry
Urinalysis

### Day 2

### During Part 1:

Vital signs: before LB-100 infusion, within 15 minutes after the end of infusion, and at 2 hours after the end of the infusion.

During Part 2:
Vital signs: before LB-100 infusion and within 15 minutes after the end of the LB-100 infusion; before the docetaxel infusion, within 15 minutes after the end of the docetaxel infusion and at 2 hours after the end of the docetaxel infusion.

### Day 3

Vital signs: before LB-100 infusion, within 15 minutes after the end of infusion, and at 2 hours after the end of the infusion.

### Days 8 and 15±3 Days

Vital signs
Hematology
Blood chemistry
Urinalysis

### Day 22 (Before Next Cycle)

Vital signs
Hematology
Blood chemistry
Urinalysis

### After Every 2 Cycles

Tumor measurements (RECIST version 1.1), tumor markers, if applicable; the same method used at baseline for a patient should be used consistently for all evaluations throughout the study. In the event PR or CR is noted, changes in tumor measurements must be confirmed by repeat assessments that should be performed at least 4 weeks after the criteria for response are first met. For SD, follow-up measurements must meet the SD criteria at least 5 weeks after study entry.

### Off-Study

ECG
Physical exam
Weight
Vital signs
ECOG performance status
Tumor measurements (RECIST version 1.1), tumor markers, if applicable
Hematology
Blood chemistry
Urinalysis
Adverse event assessment
Concomitant medications

Patients will be followed for a minimum of 30 days after last dose of study drug and followed for any unresolved adverse events considered to be related to study therapy.

### Adverse Events

An adverse event (AE) is any untoward medical occurrence in a patient or clinical investigation subject administered a pharmaceutical product that does not necessarily have a causal relationship with the treatment. An adverse event can be any unfavorable and unintended sign (including a laboratory finding), symptom or disease temporally associated with the use of a medicinal (investigational) product, whether or not related to the medicinal (investigational) product.

The AE reporting period starts on Cycle 1 Day 1 and continues through the last study visit. If an AE occurs before the first dose of study drug it will be considered a non-treatment emergent AE. At each evaluation patients should be interviewed in a non-directed manner to elicit potential adverse reactions from the patient. The occurrence of an adverse event will be based on changes in the patient's physical examination, laboratory results, and/or signs and symptoms.

All adverse events (except grade 1 and 2 laboratory abnormalities that do not require an intervention), regardless of causal relationship, are to be recorded in the case report form and source documentation. The Investigator must determine the intensity of any adverse events according to the NCI Common Terminology Criteria for Adverse Events (CTCAE) Version 4.0 (see http://ctep.info.nih.gov) and their causal relationship. Those AEs not covered by these criteria will be graded as follows:
1. Mild: Discomfort noticed, but no disruption of normal daily activity. Prescription drug not ordinarily needed for relief of symptom but may be given because of personality of patient.
2. Moderate: Discomfort sufficient to reduce or affect normal daily activity. Patient is able to continue in study; treatment for symptom may be needed.
3. Severe: Incapacitating, severe discomfort with inability to work or to perform normal daily activity. Severity may cause cessation of treatment with test drug; treatment for symptom may be given and/or patient hospitalized.
4. Life-Threatening: Symptom(s) place the patient at immediate risk of death from the reaction as it occurred; it does not include a reaction that, had it occurred in a more serious from, might have caused death.
5. Fatal: Event caused the death of the patient.

Adverse events will be followed until resolution or stabilization while the patient remains on-study. Once the patient is removed from study, events thought to be related to the study medication will be followed until resolution or stabilization, unless, in the Investigator's opinion the event is unlikely to resolve due to the patient's underlying disease, or until the patient starts a new treatment regimen or the patient is lost to follow-up.

### Attribution Definitions

An adverse event is considered to be associated with the use of the Investigational agent if the attribution is determined as possible, probable or definite. Attribution of AEs will be recorded in the CRF as:
Unrelated: The AE is clearly NOT related to the study treatment.
Unlikely: The AE is doubtfully related to the study treatment.
Possible: The AE may be related to the study treatment.
Probable: The AE is likely related to the study treatment.
Definite: The AE is clearly related to the study treatment.

### Definition of an Unexpected Adverse Event

An unexpected adverse event is defined as any adverse drug experience, the specificity or severity of which is not consistent with the current Investigator Brochure; or, if an Investigator Brochure is not required or available, the specificity or severity of which is not consistent with the risk information described in this protocol or in the regulatory agency study authorization application.

Unexpected, as used in this definition, refers to an adverse drug experience that has not been previously observed (e.g., included in the Investigator Brochure) rather than from the perspective of such experience not being anticipated from the pharmacological properties of the pharmaceutical product.

### Serious Adverse Event (SAE)

A serious adverse event is defined as any untoward medical occurrence that at any dose:
1. Results in death,
2. Is life-threatening (i.e., the patient was at risk of death at the time of the event. It does not refer to an event which hypothetically might have caused death if it was more severe),
3. Requires in-patient hospitalization or prolongation of existing hospitalization excluding that for pain management, disease staging/re-staging procedures, or catheter placement unless associated with other serious events,
4. Results in persistent or significant disability/incapacity, or
5. Is a congenital anomaly/birth defect.

Important medical events that may not result in death, be life-threatening, or require hospitalization may be considered serious adverse events when, based on appropriate medical judgment, they may jeopardize the patient or subject and may require medical or surgical intervention to prevent one of the outcomes listed above.

### Pregnancy

Any pregnancy diagnosed during the study, or that occurs within 30 days after stopping study medication, must be reported immediately to the Investigator. Pregnancy, in and of itself, is not regarded as an adverse event, unless there is suspicion that study medication may have interfered with the effectiveness of a contraceptive medication. If the patient becomes pregnant while on-study, the study drug should be immediately discontinued. Pregnancy information about a female patient or a female partner of a male patient should be reported immediately from the time the Investigator first becomes aware of a pregnancy or its outcome.

Any pregnancy complication, spontaneous abortion, elective termination of a pregnancy for medical reasons, outcome of stillbirth, congenital anomaly/birth defect, or serious adverse event in the mother will be recorded as an SAE and will be reported.

### Efficacy Assessments

Although response is not the primary endpoint of this study, patients with measurable disease will be assessed at baseline and during the study by standard criteria. Patients should be reevaluated after receiving 2 cycles of study therapy and then after every 2 cycles thereafter. In the event objective response (PR or CR) is noted, changes in tumor measurements must be confirmed by repeat assessments that should be performed at least 4 weeks after the criteria for response are first met. For stable disease (SD), follow-up measurements must meet the SD criteria at least 5 weeks after study entry.

### DEFINITIONS

Response and progression will be evaluated in this study using the international criteria (version 1.1) proposed by the Response Evaluation Criteria in Solid Tumors (RECIST) Committee [Eur J Cancer. 45 (2009) 228-247]. Changes in only the largest diameter (unidimensional measurement) of the tumor lesions are used in the RECIST 1.1 guidelines. Note: Lesions are either measurable or non-measurable using the criteria provided below. The term “evaluable” in reference to measurability will not be used because it does not provide additional meaning or accuracy.

### Measurable Disease

Measurable disease is defined by the presence of at least one measurable lesion. Measurable lesions are defined as those that can be accurately measured in at least one dimension [longest diameter (LD) in the plane of measurement to be recorded] with a minimum size of:
10 mm by CT scan (CT scan slice thickness no greater than 5 mm)
10 mm caliper measurement by clinical exam (lesions which cannot be accurately measured with calipers should be recorded as non-measurable)
20 mm by chest x-ray

Malignant lymph nodes: To be considered pathologically enlarged and measurable, a lymph node must be ≧15 mm in short axis when assessed by CT scan (CT scan slice thickness no greater than 5 mm).

### Non-measurable Disease

All other lesions (or sites of disease), including small lesions (longest diameter <10 mm or pathological lymph nodes with ≧10 to <15 mm short axis) are considered non-measurable disease. Lesions considered truly non-measurable include: leptomeningeal disease, ascites, pleural/pericardial effusions, lymphangitis cutis/pulmonis, inflammatory breast disease, abdominal masses/abdominal organomegaly identified by physical exam and not followed by CT or MRI.

Bone lesions, cystic lesions and lesions previously treated with local therapy must be considered as follows:

Bone lesions:
Bone scan, PET scan or plain films are not considered adequate imaging techniques to measure bone lesions. However, these techniques can be used to confirm the presence or disappearance of bone lesions.
Lytic bone lesions or mixed lytic-blastic lesions, with identifiable soft tissue components, that can be evaluated by cross sectional imaging techniques (i.e., CT or MRI) can be considered as measurable lesions if the soft tissue component meets the definition of measurability described above.
Blastic bone lesions are non-measurable.

Cystic lesions:
Lesions that meet the criteria for radiographically defined simple cysts should not be considered malignant lesions (neither measurable or non-measurable) since they are, by definition, simple cysts.
‘Cystic lesions’ thought to represent cystic metastases can be considered measurable lesions, if they meet the definition of measurability described above. However, if non-cystic lesions are present in the same patient, these are preferred for selection as target lesions.

Lesions with prior local treatment:
Tumor lesions situated in a previously irradiated area, or in an area subjected to other loco-regional therapy, are usually not considered measurable unless there has been demonstrated progression in the lesion.

### Target Lesions

All measurable lesions up to a maximum of two lesions per organ and five lesions in total, representative of all involved organs, should be identified as target lesions and recorded and measured at baseline. Target lesions should be selected on the basis of their size (lesions with the longest diameter) and their suitability for accurate repeated measurements (either by imaging techniques or clinically). A sum of the diameters (longest for non-nodal lesions, short axis for nodal lesions) for all target lesions will be calculated and reported as the baseline sum diameters. The baseline sum diameters will be used as reference by which to characterize the objective tumor response.

### Lymph Node Assessment

For lymph nodes, measurements should be made of the short axis, which is defined as perpendicular to the LD of node assessed in the plane of measurement:
Target lesion if short axis ≧15 mm
Non-target lesion if short axis is ≧10 but <15 mm
Normal if short axis <10 mm

For baseline, add the actual short axis measurement to the sum of LD of non-nodal lesions.

### Non-Target Lesions

All other lesions (or sites of disease) including pathological lymph nodes should be identified as non-target lesions and should also be recorded at baseline. Measurements of these lesions are not required and these lesions should be followed as “present,” “absent,” or in rare cases “unequivocal progression.” In addition, it is possible to record multiple non-target lesions involving the same organ as a single item on the case report form (e.g., ‘multiple enlarged pelvic lymph nodes’ or ‘multiple liver metastases’).

Guidelines for Evaluation of Measurable Disease

All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination when both methods have been used to assess the antitumor effect of a treatment.

### Clinical Lesions.

Clinical lesions will only be considered measurable when they are superficial and ≧10 mm diameter as assessed using calipers (e.g., skin nodules). In the case of skin lesions, documentation by color photography, including a ruler to estimate the size of the lesion, is recommended. When lesions can be evaluated by both clinical exam and imaging, imaging evaluation should be undertaken since it is more objective and may be reviewed at the end of the study.

### Chest X-Ray.

Chest CT is preferred over chest x-ray, particularly when progression is an important endpoint. Lesions on chest x-ray may be considered measurable if they are clearly defined and surrounded by aerated lung.

### Conventional CT and MRI.

This guideline has defined measurability of lesions on CT scan based on the assumption that CT slice thickness is 5 mm or less. When CT scans have slice thickness >5 mm, the minimum size for a measurable lesion should be twice the slice thickness. MRI is acceptable in certain situations (e.g., for body scans).

### Ultrasound (US).

US should not be used to measure tumor lesions. US examinations cannot be reproduced in their entirety for independent review at a later date because they are operator dependent. If new lesions are identified by US, confirmation by CT or MRI is advised. If there is concern about radiation exposure at CT, MRI may be used instead of CT.

### Endoscopy, Laparoscopy.

The utilization of these techniques for objective tumor evaluation is not advised. However, such techniques can be useful to confirm complete pathological response when biopsies are obtained or to determine relapse in trials where recurrence following complete response or surgical resection is an endpoint.

### Tumor Markers.

Tumor markers alone cannot be used to assess objective tumor response. If markers are initially above the upper normal limit, they must normalize for a patient to be considered in complete clinical response.

### Cytology, Histology.

These techniques can be used to differentiate between partial responses (PR) and complete responses (CR) in rare cases (e.g., residual lesions in tumor types such as germ cell tumors, where known residual benign tumors can remain).

### Response Criteria

Complete Response (CR): Disappearance of all target lesions. Any pathological lymph nodes (whether target or non-target) must have reduction in short axis to <10 mm.
Partial Response (PR): At least a 30% decrease in the sum of diameters of target lesions, taking as reference the baseline sum diameters.
Progressive Disease (PD): At least a 20% increase in the sum of diameters of target lesions, taking as reference the smallest sum on study (this includes the baseline sum if that is the smallest). In addition to the relative increase of 20%, the sum must also demonstrate an absolute increase of at least 5 mm. The appearance of one or more new lesions is also considered progression.
Stable Disease (SD): Neither sufficient shrinkage to qualify for PR nor sufficient increase to qualify for PD, taking as reference the smallest sum diameters while on study.

### Assessment of Target Lymph Nodes

Lymph nodes identified as target lesions should always have the actual short axis measurement recorded (measured in the same anatomical plane as the baseline exam), even if the nodes regress to below 10 mm on study. In order to qualify for CR, each node must achieve a short axis <10 mm. For PR, SD and PD, the actual short axis measurement of the nodes is to be included in the sum of target lesions.

Target Lesions that Become “Too Small to Measure”

All lesions (nodal and non-nodal) recorded at baseline should have their actual measurements recorded at each subsequent evaluation, even when very small (e.g., 2 mm). If it is the opinion of the radiologist that the lesion has disappeared, the measurement should be recorded as 0 mm. If the lesion is believed to be present and is faintly seen but too small to measure, a default value of 5 me should be assigned.

Lesion that Split or Coalesce on Treatment

When non-nodal lesions fragment, the longest diameters of the fragmented portions should be added together to calculate the target lesion sum. Similarly, as lesions coalesce, a plane between them may be maintained that would aid in obtaining diameter measurements of each individual lesion. If the lesions have truly coalesced such that they are no longer separable, the vector of the longest diameter should be the maximal longest diameter for the ‘coalesced lesion.’

### Evaluation of Non-Target Lesions

Complete Response (CR): Disappearance of all non-target lesions and normalization of tumor marker level. All lymph nodes must be non pathological in size (<10 mm short axis).
Non-CR/Non-PD: Persistence of one or more non-target lesion(s) and/or maintenance of tumor marker level above the normal limits.
Progressive Disease (PD): Unequivocal progression of existing non-target lesions. (The appearance of one or more new lesions is also considered progression.) To achieve ‘unequivocal progression’ on the basis of the non-target disease, there must be an overall level of substantial worsening in non-target disease such that, even in the presence of SD or PR in target disease, the overall tumor burden has increased sufficiently to merit discontinuation.

### New Lesions

The finding of a new lesion should be unequivocal (i.e., not attributed to differences in scanning technique, change in imaging modality, or findings thought to represent something other than tumor, such as a ‘new’ healing bone lesion). A lesion identified on a follow-up study in an anatomical location that was not scanned at baseline is considered a new lesion and will indicate disease progression. If a new lesion is equivocal, continued therapy and follow-up evaluation will clarify if it represents truly new disease. If repeat scans confirm this is definitely a new lesion, then progression should be declared using the date of the initial scan.

### Evaluation of Best Overall Response

The best overall response is the best response recorded from the start of the treatment until disease progression/recurrence (taking as reference for progressive disease the smallest measurements recorded since the treatment started). The patient's best overall response assignment will depend on findings of both target and non-target disease and will also take into consideration the appearance of new lesions. It is assumed that at each protocol-specified time point, a response assessment occurs. Table provides a summary of the overall response status calculation at each time point for patients who have measurable disease at baseline.

TABLE 3
Time point response: Patients with target (+/−non-target)
disease

| Target Lesions | Non-target Lesions | New Lesions | Overall Response |
|---|---|---|---|
| CR | CR | No | CR |
| CR | Non-CR/non-PD | No | PR |
| CR | Not evaluated | No | PR |
| PR | Non-PD or not all | No | PR |
| | evaluated | | |
| SD | Non-PD or not all | No | SD |
| | evaluated | | |
| Not all evaluated | Non-PD | No | NE |
| PD | Any | Yes or No | PD |
| Any | PD | Yes or No | PD |
| Any | Any | Yes | PD |

CR = complete response,
PR = partial response,
SD = stable disease
PD = progressive disease,
NE = inevaluable

Complete or partial responses may be claimed only if the criteria for each are confirmed by a repeat assessment at least 4 weeks later. In this circumstance, the best overall response can be interpreted as in Table.

TABLE 4
Best overall response when confirmation of CR and PR
required

| Overall | | |
|---|---|---|
| response | Overall response | |
| First time | Subsequent time | |
| point | point | BEST overall response |
| CR | CR | CR |
| CR | PR | SD, PD or PR* |
| CR | SD | SD provided minimum criteria for SD |
| | | duration met, otherwise PD |
| CR | PD | SD provided minimum criteria for SD |
| | | duration met, otherwise PD |
| CR | NE | SD provided minimum criteria for SD |
| | | duration met, otherwise NE |
| PR | CR | PR |
| PR | PR | PR |
| PR | SD | SD |
| PR | PD | SD provided minimum criteria for SD |
| | | duration met, otherwise PD |
| PR | NE | SD provided minimum criteria for SD |
| | | duration met, otherwise NE |
| NE | NE | NE |

CR = complete response;
PR = partial response;
SD = stable disease;
PD = progressive disease;
NE = inevaluable
*If CR is truly met at first time point, then any disease seen at a subsequent time point, even disease meeting PR criteria relative to baseline, makes the disease PD at that point (since disease must have reappeared after CR). Best response would depend on whether minimum duration for SD was met. However, sometimes ‘CR’ may be claimed when subsequent scans suggest small lesions were likely still present and in the fact patient had PR, not CR, at the first time point. Under these circumstances, the original CR should be changed to PR and the best response is PR.

### Confirmation

To be assigned a status of PR or CR, changes in tumor measurements must be confirmed by repeat assessments that should be performed at least 4 weeks after the criteria for response are first met. In the case of SD, follow-up measurements must have met the SD criteria at least once after study entry at a minimum interval at 5 weeks.

### Duration of Overall Response

The duration of overall response is measured from the time measurement criteria are first met for CR or PR (whichever is first recorded) until the first date that recurrent or progressive disease is objectively documented (taking as reference for progressive disease the smallest measurements recorded since the treatment started) or death, whichever occurs first.

The duration of overall CR is measured from the time measurement criteria are first met for CR until the first date that recurrent disease is objectively documented.

### Duration of Stable Disease

Stable disease is measured from the start of the treatment until the criteria for progression are met, taking as reference the smallest measurements recorded since the treatment started. In the case of SD, follow-up measurements must have met the SD criteria at least once after study entry at a minimum interval at 5 weeks.

### Pharmacokinetics

### Pharmacokinetic Sample Collection—Part 1

Plasma sampling for pharmacokinetic (PK) measurements of LB-100 will be performed in Cycle 1 for the 3 patients in the MTD confirmation cohort. Blood samples will be collected on Cycle 1 Days 1 and 3 as per the schedule in Table. At each timepoint, 5 mL will be drawn into a chilled heparin collection tube and kept on ice until the plasma is separated and frozen at −70° C.

Procedures for the processing, storage and shipment of the samples are located in the Study Operations Manual.

TABLE 5
Pharmacokinetic Sampling for Part 1

| | | One (1) |
|---|---|---|
| Study | | 5 mL Heparin |
| Day | Draw Time | for LB-100 |
| Day 1 | pre-dose | X |
| | immediately at end of LB-100 infusion | X |
| | 15 minutes (±2 minutes) post LB-100 infusion | X |
| | 30 minutes (±2 minutes) post LB-100 infusion | X |
| | 1 hour (±5 minutes) post LB-100 infusion | X |
| | 2 hours (±5 minutes) post B-100 infusion | X |
| | 4 hours (±5 minutes) post LB-100 infusion | X |
| Day 3 | pre-dose | X |
| | immediately at end of LB-100 infusion | X |
| | 15 minutes (±2 minutes) post LB-100 infusion | X |
| | 30 minutes (±2 minutes) post LB-100 infusion | X |
| | 1 hour (±5 minutes) post LB-100 infusion | X |
| | 2 hours (±5 minutes) post LB-100 infusion | X |
| | 4 hours (±5 minutes) post LB-100 infusion | X |

### Pharmacokinetic Sample Collection—Part 2

Plasma sampling for PK measurements of LB-100 will be performed in Cycle 1 for the 3 patients in the MTD/RD confirmation cohort. Blood samples will be collected on Cycle 1 Days 1 and 3 as per the schedule in Table. At each timepoint, 5 mL will be drawn into a chilled heparin collection tube for LB-100 analysis. Collection tubes will be kept on ice until the plasma is separated and frozen at −70° C.

Procedures for the processing, storage and shipment of the samples are located in the Study Operations Manual.

TABLE 6
Pharmacokinetic Sampling for Part 2

| | | One (1) 5 mL |
|---|---|---|
| Study | | Heparin for |
| Day | Draw Time | LB-100 |
| Day 1 | pre-dose | X |
| | immediately at end of LB-100 infusion | X |
| | 15 minutes (±2 minutes) post LB-100 infusion | X |
| | 30 minutes (±2 minutes) post LB-100 infusion | X |
| | 1 hour (±5 minutes) post LB-100 infusion | X |
| | 2 hours (±5 minutes) post LB-100 infusion | X |
| | 4 hours (±5 minutes) post LB-100 infusion | X |
| Day 3 | pre-dose | X |
| | immediately at end of LB-100 infusion | X |
| | 15 minutes (±2 minutes) post LB-100 infusion | X |
| | 30 minutes (±2 minutes) post LB-100 infusion | X |
| | 1 hour (±5 minutes) post LB-100 infusion | X |
| | 2 hours (±5 minutes) post LB-100 infusion | X |
| | 4 hours (±5 minutes) post LB-100 infusion | X |

### Statistics

Demographic data will be displayed and summary statistics will be used to describe the study population (e.g., ranges, mean and medians for age, weight, and height; numbers of males and females; description of baseline performance status and disease characteristics). Safety and efficacy data will be tabulated. In general, statistical analyses will be descriptive.

### Primary Endpoints

For Parts 1 and 2 of the study, the primary endpoints are the number of patients with DLTs, toxicity, and the MTD and, in Part 2, the recommended Phase II dose (RD).

### Secondary Endpoint

For Parts 1 and 2 of the study, the secondary endpoints are best overall response, objective response rate (CR or PR), duration of response, and the determination of PK parameters for LB-100 when given alone and when given in combination with docetaxel.

### Analysis Populations

The following analysis populations will be defined:

Intent-to-Treat (ITT) Population: All patients registered in the study whether or not they met the eligibility criteria or received study drug will be included in the ITT population. This population will be summarized for demographics and other baseline characteristics, and serve as the secondary analysis population for efficacy.

Safety Evaluable Population: All patients who received any amount of s LB-100 will be considered evaluable for safety. This population will be summarized for all safety parameters.

Efficacy Evaluable Population: All patients who met the eligibility criteria with baseline measurable disease, completed 2 cycles of study therapy, and had at least one post-baseline tumor assessment will be considered evaluable for efficacy. For patients with less than 2 cycles of study therapy, there must be clear evidence of clinical progression to be considered evaluable for efficacy which includes symptomatic deterioration or death due to any cause. The efficacy-evaluable population will be the primary analysis population for efficacy.

### Safety Analysis

Safety data will be tabulated for the safety population. These data will include adverse events, vital signs, and laboratory parameters. Adverse events will be tabulated by body system, preferred term, severity, and relationship to treatment. For the purpose of presentation, groups will be arranged by decreasing frequency of adverse events. The tabulation of laboratory parameters will include the normal ranges for each parameter. Each value will be classified as falling above, below, or within the normal range. Laboratory parameters will also be tabulated by maximum NCI-CTCAE severity grade. Adverse event terms will be coded using the Medical Dictionary for Drug Regulatory Activities (MedDRA®).

### Efficacy Analysis

Best overall response will be assessed using RECIST version 1.1. Frequency counts and percentages will be presented for the tumor response categories defined by RECIST. The objective overall antitumor response rate (CR or PR) will be defined as the proportion of patients with confirmed response. The overall response rate and its 95% confidence interval will be calculated. For duration of response, life table estimates will be calculated using Kaplan-Meier methodology and the 95% confidence interval will be calculated for the median time For duration of response, a patient alive with no disease progression will be censored at the date of the last evaluable tumor assessment.

### Pharmacokinetic Analysis

Plasma concentrations and calculated pharmacokinetic parameters (maximum concentration, time to reach maximum concentration, area under the curve, half life, total body clearance, and volume of distribution) will be determined for LB-100 as a single agent (Part 1), and when given in combination with docetaxel (Part 2).

### Sample Size

Approximately 42 patients will be enrolled in the study. Of these, approximately 18 patients will be enrolled in Part 1 and 18 patients in Part 2. An additional 6 patients (3 in each Part) will be enrolled in the MTD confirmation cohorts. The number of patients is not based on statistical considerations.

### Example 1

### Clinical Dosing of LB-100 (Single Agent)

LB-100 was administered to patients 1-14 and 16-17 in doses ranging from 0.5 mg/m² to 1.75 mg/m² (see Table 7). There was no dose limiting toxicity in any of the patients and no progression of disease was observed in patients 9, 10, 12-14 and 16-17.

Patient 10 was diagnosed with pancreatic cancer and received 8 cycles of LB-100. At end of cycle 8, imaging studies showed a stable mass in the pancreas and small lung nodules. Cycle 9 of LB-100 treatment was commenced. Patient 10 has pancreatic cancer that was progressing despite several rounds of different chemotherapy regimens and since starting LB-100 has remained stable for 6 months.

Patient 12 was diagnosed with metastatic breast cancer and received 4 Cycles of LB-100. Grade 1 neuropathy, possibly related to LB-100, has been observed.

Patient 13 was diagnosed with metastatic testicular cancer, and has begun Cycle 4 of treatment. No adverse events attributable to LB-100 have been reported.

Patient 14 was diagnosed with ovarian cancer and has undergone one full cycle and one partial cycle. No new adverse events have been observed.

Patient 16 was diagnosed with colon cancer and has completed one cycle of treatment. The patient has developed grade 3 anemia and grade 2 weakness that is attributed to a bleeding rectal mass (grade 3). Patient will continue treatment following radiation therapy.

Patient 17 was diagnosed with metastatic thymoma and began treatment. The patient is tolerating treatment (in middle of cycle 1) without any symptoms attributable to LB-100.

TABLE 7
Analysis of Patient Cohorts.

| | Tumor | Date | Dose | Cycles | | Dose Limiting |
|---|---|---|---|---|---|---|
| Patient | Type | Enrolled | (Cohort) | Received | Patient Status | Toxicity? |
| 1 | Uterine | 19 Apr. 2013 | 0.25 mg/m² | 2 | Off-study 3 JUN. 2013 - | No |
| | | | | | Progressive disease | |
| 2 | Sarcoma | 16 May 2013 | 0.25 mg/m² | 2 | Off-study 1 JUL. 2013 - | No |
| | | | | | Progressive disease | |
| 3 | Duodenal | 17 Jun. 2013 | 0.25 mg/m² | 3 | Off-study 29 JUL. 2013 - | No |
| | | | | | Progressive disease | |
| 4 | Appendiceal | 7 Aug. 2013 | 0.5 mg/m² | 2 | Off-study 20 SEP. 2013 - | No |
| | | | | | Progressive disease | |
| 5 | NSCLC | 1 Nov. 2013 | 0.5 mg/m² | 2 | Off-study 19 JAN. 2014 - | No |
| | | | | | Progressive disease after | |
| | | | | | 3 cycles of treatment | |
| 6 | Colon | 24 Jan. 2014 | 0.5 mg/m² | 2 | On study 24 MAR. 2014 - | No |
| | | | | | clinical deterioration | |
| 7 | Atypical | 18 Feb. 2014 | 0.83 mg/m² | 0 | Off-study after 1 dose | N/A - not |
| | carcinoid | | | | of LB-100 (fever & | evaluable |
| | of the lung | | | | pneumonia unrelated | |
| | | | | | to LB-100) | |
| 8 | Colon | 24 Mar. 2014 | 0.83 mg/m² | 2 | Off-study after 2 cycles, | No |
| | | | | | progressive disease | |
| | | | | | 6 MAY 2014 | |
| 9 | Atypical | 16 Apr. 2014 | 0.83 mg/m² | 5 | Off treatment after | No |
| | carcinoid | | | | Cycle 5; 11 AUG. 2014 | |
| | of the lung | | | | | |
| 10 | Pancreas | 6 Aug. 2014 | 0.83 mg/m² | 8 | Begin Cycle 8, 7 JAN. 2015 | No |
| 11 | Colon | 20 Oct. 2014 | 1.25 mg/m² | 1 | Off treatment after | No |
| | | | | | cycle 1 on 11 NOV. 2014 | |
| 12 | Breast | 17 Nov. 2014 | 1.25 mg/m² | 4 | To begin cycle 4 | No |
| 13 | Testicular | 5 Dec. 2014 | 1.25 mg/m² | 3 | Tobegin Cycle 4 | No |
| 14 | Ovarian | 29 Dec. 2014 | 1.75 mg/m² | 1 | Cycle 2 in progress | No |
| 16 | Colon | 29 Dec. 2014 | 1.75 mg/m² | 1 | Cycle 2 delayed until | No |
| | | | | | palliative RT has been | |
| | | | | | administered | |
| 17 | Tymoma | 28 Jan. 2015 | 1.75 mg/m² | 0 | Cycle 1 in progress | No |

Doses of LB-100 in patients with pancreatic cancer, testicular cancer and breast cancer in an amount of 0.83 mg/m², 1.25 mg/m² or 1.75 mg/m² have shown no dose limiting toxicity and have stabilized the cancer.

Doses of LB-100 in amounts of from 3.0 mg/m² to 4.5 mg/m² are useful in treating cancer alone or in combination with docetaxel in amounts 10 of 60 mg/m² or 75 mg/m². Doses of LB-100 in amounts of from 3.0 mg/m² to 4.5 mg/m² show no dose limiting toxicity and stabilize the cancer when administered alone or in combination with docetaxel in amounts of 60 mg/m² or 75 mg/m².

TABLE 8
Updated Analysis of Patient Cohorts.

| | Tumor | Date | Dose | Cycles | | Dose Limiting |
|---|---|---|---|---|---|---|
| Patient | Type | Enrolled | (Cohort) | Received | Patient Status | Toxicity? |
| 1 | Uterine | 19 Apr. 2013 | 0.25 mg/m² | 2 | Off-study 3 JUN. 2013 - | No |
| | | | | | Progressive disease | |
| 2 | Sarcoma | 16 May 2013 | 0.25 mg/m² | 2 | Off-study 1 JUL. 2013 - | No |
| | | | | | Progressive disease | |
| 3 | Duodenal | 17 Jun. 2013 | 0.25 mg/m² | 3 | Off-study 29 JUL. 2013 - | No |
| | | | | | Progressive disease | |
| 4 | Appendiceal | 7 Aug. 2013 | 0.5 mg/m² | 2 | Off-study 20 SEP. 2013 - | No |
| | | | | | Progressive disease | |
| 5 | NSCLC | 1 Nov. 2013 | 0.5 mg/m² | 2 | Off-study 19 JAN. 2014 - | No |
| | | | | | Progressive disease after | |
| | | | | | 3 cycles of treatment | |
| 6 | Colon | 24 Jan. 2014 | 0.5 mg/m² | 2 | On study 24 MAR. 2014 - | No |
| | | | | | clinical deterioration | |
| 7 | Atypical | 18 Feb. 2014 | 0.83 mg/m² | 0 | Off-study after 1 dose | N/A - not |
| | carcinoid | | | | of LB-100 (fever & | evaluable |
| | of the lung | | | | pneumonia unrelated | |
| | | | | | to LB-100) | |
| 8 | Colon | 24 Mar. 2014 | 0.83 mg/m² | 2 | Off-study after 2 cycles, | No |
| | | | | | progressive disease | |
| | | | | | 6 MAY 2014 | |
| 9 | Atypical | 16 Apr. 2014 | 0.83 mg/m² | 5 | Off treatment 11 AUG. | No |
| | carcinoid | | | | 2014. Best response: | |
| | of the lung | | | | stable disease | |
| 10 | Pancreas | 6 Aug. 2014 | 0.83 mg/m² | 16 | Off-study 17 JUN. | No |
| | | | | | 2015; Best response: | |
| | | | | | stable disease, 39 | |
| | | | | | weeks duration. | |
| 11 | Colon | 20 Oct. 2014 | 1.25 mg/m² | 2 | Off treatment 11 NOV. | No |
| | | | | | 2014. Progressive | |
| | | | | | disease. | |
| 12 | Breast | 17 Nov. 2014 | 1.25 mg/m² | 4 | Off treatment 17 FEB. | No |
| | | | | | 2015; Best response: | |
| | | | | | stable disease | |
| 12 | Breast | 17 Nov. 2014 | 1.25 mg/m² | 4 | Off treatment 17 FEB. | No |
| | | | | | 2015; Best response: | |
| | | | | | stable disease | |
| 13 | Testicular | 5 Dec. 2014 | 1.25 mg/m² | 5 | Off treatment 2 MAR. | No |
| | | | | | 2015; Best response: | |
| | | | | | stable disease | |
| 14 | Ovarian | 29 Dec. 2014 | 1.75 mg/m² | 6 | Off treatment 6 MAY | No |
| | | | | | 2015; Best response: | |
| | | | | | stable disease | |
| 16 | Colon | 29 Dec. 2014 | 1.75 mg/m² | 2 | Off treatment, | No |
| | | | | | progressive disease | |
| 17 | Thymoma | 28 Jan. 2015 | 1.75 mg/m² | 8 | treatment, stable disease | No |
| 18 | Colon | 2 Mar. 2015 | 2.33 mg/m² | 0 | Off treatment after 2 | N/A-not |
| | | | | | doses due to elevated | evaluable |
| | | | | | serum creatinine | |
| 19 | Vulvar | 19 May 2015 | 2.33 mg/m² | 1 | Off treatment, | No |
| | | | | | progressive disease | |
| 10 | NSCLC | 12 Jun. 2015 | 2.33 mg/m² | 0 | Off study due to pulmonary | N/A-Not |
| | | | | | embolism before completing | evaluable |
| | | | | | cycle 1. | |
| 21 | Ovarian | 22 Jun. 2015 | 2.33 mg/m² | 0 | On treatment, too early to | No |
| | | | | | assess response. | |
| 22 | NSCLC | 7 Jul. 2015 | 2.33 mg/m² | 0 | Started treatment 8 JUL. | N/A-Not |
| | | | | | 2015, too early to | evaluable |
| | | | | | assess DLT or response | |

LB-100 was also administered to patients 18-22 in doses of 2.33 mg/m² (see Table 8).

The starting dose of LB-100 was 0.25 mg/m² and twenty one patients have enrolled in part 1 of the study through six dose levels. Six patients received 2.33 mg/m² and no DLTs have been observed. One patient with metastatic colon cancer at DL6 (2.33 mgm²) had a grade 2 creatinine after 2 doses that resolved with hydration. This was related to LB-100 and the study was amended to increase the volume and infusion time.

The amended protocol includes adding LB100 to 500 mL of normal saline (0.9%) and infusing over two hours instead of placing the LB-100 in 50 ml of saline and infusing over 15 minutes.

One patient on DL3 (0.83 mgm²) with stage 4 pancreas cancer received at least 15 cycles of treatment with stabilization of disease. Another patient on DL5 (1.75 mgm²) with metastatic thymoma remains on treatment through 7 cycles with stable disease. Stable disease was observed in breast, ovarian, carcinoid and testicular cancer patients.

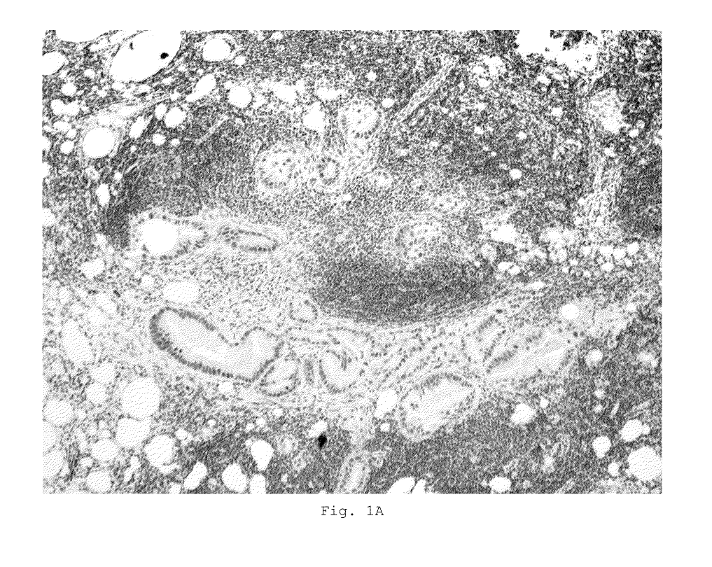

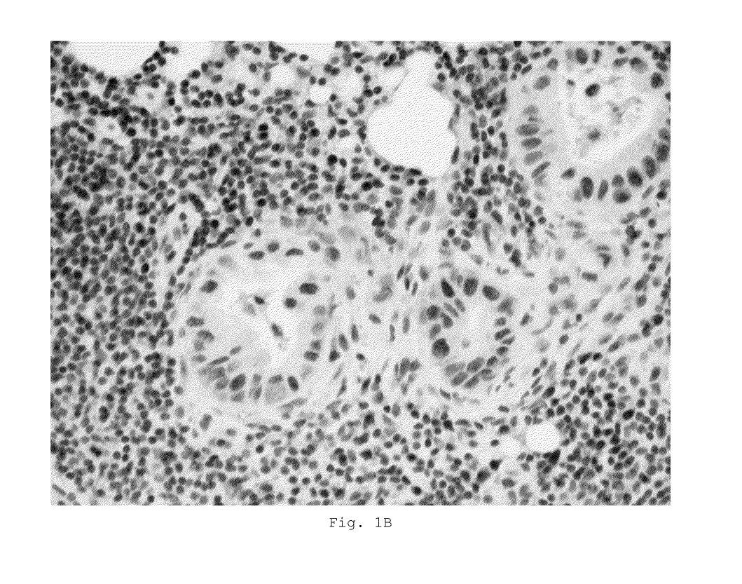

Patient 10 was diagnosed with pancreatic cancer. The pancreatic cancer was stable for 15 cycles of LB-100 (45 weeks) was shown to markedly overexpress MAD2 in most of the cancer cells (FIGS. 1A-B). The patient was treated with LB-100 at 0.83 mg/m² daily for three days every 3 weeks time 15 three week cycles and had no toxicity.

Patient 17 was diagnosed with metastatic thymoma and began treatment. The patient is tolerating treatment (in middle of cycle 8) with only mild (grade 1) dyspnea. Patient has stable disease.

Patient 20 was diagnosed with Non-squamous NSCLC and received less than 1 cycle of LB-100. The patient began cycle 1 of LB-100 as the 2nd patient enrolled to Cohort 6. The patient is off-study due to complications associated with a pulmonary embolism (PE). The investigator determined the PE was not related to treatment with LB-100

Patient 21 was diagnosed with ovarian carcinoma and has received less than 1 cycle of LB-100. During her first cycle of LB-100 treatment the patient development grade 3 vomiting on day 3 of treatment with LB-100, assessed by the treating physician as related to LB-100. It was determined that this adverse event did not meet dose-limiting toxicity criteria because maximum antiemetic treatment had not been given. No other adverse events are reported.

Patient 22 was diagnosed with Non-small cell lung cancer, and has received less than 1 cycle of LB-100. No adverse events attributable to LB-100 have been reported.

### Example 2

### Tumors Overexpressing N-CoR

LB-100 inhibited PP2A activity in tumors overexpressing N-CoR, e.g. glioblastoma multiforme (GBM). LB-100 also inhibited GBM cell growth. See, for example, US 2009/0018142 A9.

LB-100 inhibits PP2A in human subjects afflicted with glioblastoma multiforme (GBM) and is useful in treating human subjects afflicted with glioblastoma multiforme (GBM) when administered to the subject in an amount of from 0.25 mg to 7.5 mg or an amount from 0.1 mg/m² to 5.0 mg/m².

### Example 3

### Other Cancers

LB-100 inhibited PP2A activity and inhibited cell growth of breast cancer cells, large cell lung cancer cells, lung adenocarcinoma cells, small cell lung cancer cells, stomach cancer cells, liver cancer cells, ovarian cancer cells, pancreatic cancer cells, prostate cancer cells, promyelocytic leukemia cells, acute lymphoma cells and chronic myelogenous leukemia (CML) cells. See, for example, U.S. Pat. No. 7,998,957 B2, U.S. Pat. No. 8,227,473 B2. LB-100 also inhibited glioblastoma multiforme (U87) and medulloblastoma (DAOY) tumor growth in mice. See, for example, US 2009/0035292 A1 and US 2010/0029640 A1.

LB-100 inhibits PP2A in human subjects afflicted with breast cancer, large cell lung cancer, lung adenocarcinoma, small cell lung cancer, stomach cancer, liver cancer, ovarian cancer, pancreatic cancer, prostate cancer, promyelocytic leukemia, acute lymphoma, chronic myelogenous leukemia (CML), glioblastoma multiforme or medulloblastoma and is useful in treating human subjects afflicted with breast cancer, large cell lung cancer, lung adenocarcinoma, small cell lung cancer, stomach cancer, liver cancer, ovarian cancer, pancreatic cancer, prostate cancer, promyelocytic leukemia, acute lymphoma chronic myelogenous leukemia (CML), glioblastoma multiforme or medulloblastoma when administered to the subject in an amount of from 0.25 mg to 7.5 mg or an amount from 0.1 mg/m² to 5.0 mg/m².

### Example 4

### Combination with Cisplatin, Doxorubicin, Taxol or Temozolomide

LB-100 enhanced the cytotoxic effects of each of cisplatin, doxorubicin, taxol and temozolomide when simultaneously exposed to the human glioblastoma cell line, U373. See, for example, US 2010/0029683 A1.

LB-100 inhibits PP2A in human subjects afflicted with glioblastoma multiforme (GBM) and is useful in treating human subjects afflicted with GBM when administered to the subject alone in an amount of from 0.25 mg to 7.5 mg or an amount from 0.1 mg/m² to 5.0 mg/m² or when administered in the aforementioned amounts in combination with cisplatin, doxorubicin, taxol or temozolomide.

### Example 5

### Triple-Negative Breast Cancer (TNBC)

LB-100 inhibits PP2A activity and is cytotoxic to TNBC cells. LB-100 synergizes with chemotherapeutic drugs to enhance cytotoxicity against TNBC cells. LB-100 also enhances the effects of chemotherapy in TNBC xenografts. LB-100 chemosensitizes doxorubicin and cisplatin to TNBC cells and/or has a synergistic cytotoxic effect with doxorubicin and/or cisplatin against TNBC cells.

Both doxorubicin (0.2 μg/ml) and cisplatin (2 μg/ml) in combination with LB-100 are cytotoxic to TNBC cell lines. The addition of LB-100 sensitizes TNBC cells to doxorubicin and cisplatin. TNBC cell lines include HCC1599, HCC1937, HCC1599, MDA-MB-468, HCC38, HCC70, HCC1806, HCC1187, DU4475, BT-549, Hs-578T, MDA-MB-231, MDA-MB-436, MDA-MB-157, MDA-MB-453, BT-20, and HCC1395.

The combination of doxorubicin (1.5 mg/kg) or cisplatin (3 mg/kg) and LB-100 (2 mg/kg, qod or 2.5 mg/kg, respectively) significantly slows the growth of TNBC tumors in mice with reduction of tumor volume with no or less effect on tumor growth in animals treated with single agents.

An amount of compound LB-100 in combination with a chemotherapeutic agent is administered to a subject afflicted with triple-negative breast cancer. The amount of the compound is effective to enhance the anti-cancer activity of the chemotherapeutic agent.

An amount of compound LB-100 in combination with cisplatin or doxorubicin is administered to a subject afflicted with triple-negative breast cancer. The amount of the compound is effective to enhance the anti-cancer activity of the cisplatin or doxorubicin.

An amount of compound LB-100 in combination with sorafenib is administered to a subject afflicted with triple-negative breast cancer. The amount of the compound is effective to enhance the anti-cancer activity of the sorafenib.

LB-100 has an analogous effect in bladder cancer, cervical cancer, malignant mesothelioma, non-small cell lung cancer, stomach cancer and ovarian cancer models when compared to TNBC models. LB-100 chemosensitizes doxorubicin and cisplatin to bladder cancer, cervical cancer, malignant mesothelioma, non-small cell lung cancer, stomach cancer and ovarian cancer cells and/or has a synergistic cytotoxic effect with doxorubicin and/or cisplatin against bladder cancer, cervical cancer, malignant mesothelioma, non-small cell lung cancer, stomach cancer and ovarian cancer cells.

Both doxorubicin (0.2 μg/ml) and cisplatin (2 μg/ml) in combination with LB-100 are cytotoxic to bladder cancer, cervical cancer, malignant mesothelioma, non-small cell lung cancer, stomach cancer and ovarian cancer cell lines. The addition of LB-100 sensitizes the cancer cells to doxorubicin and cisplatin.

The combination of doxorubicin (1.5 mg/kg) or cisplatin (3 mg/kg) and LB-100 (2 mg/kg, qod or 2.5 mg/kg, respectively) significantly slows the growth of bladder cancer, cervical cancer, malignant mesothelioma, non-small cell lung cancer, stomach cancer and ovarian cancer tumors in mice with reduction of tumor volume with no or less effect on tumor growth in animals treated with single agents.

An amount of compound LB-100 in combination with a chemotherapeutic agent is administered to a subject afflicted with bladder cancer, cervical cancer, malignant mesothelioma, non-small cell lung cancer, stomach cancer or ovarian cancer. The amount of the compound is effective to enhance the anti-cancer activity of the chemotherapeutic agent.

An amount of compound LB-100 in combination with cisplatin or doxorubicin is administered to a subject afflicted with bladder cancer, cervical cancer, malignant mesothelioma, non-small cell lung cancer, stomach cancer or ovarian cancer. The amount of the compound is effective to enhance the anti-cancer activity of the cisplatin or doxorubicin.

An amount of compound LB-100 in combination with sorafenib is administered to a subject afflicted with bladder cancer, cervical cancer, malignant mesothelioma, non-small cell lung cancer, stomach cancer or ovarian cancer. The amount of the compound is effective to enhance the anti-cancer activity of the sorafenib.

LB-100 inhibits PP2A in human subjects afflicted with TNBC and is useful in treating human subjects afflicted with TNBC when administered to the subject alone in an amount of from 0.25 mg to 7.5 mg or an amount from 0.1 mg/m² to 5.0 mg/m² or when administered in the aforementioned amounts in combination with cisplatin, doxorubicin, or sorafenib.

LB-100 inhibits PP2A in human subjects afflicted with bladder cancer, cervical cancer, malignant mesothelioma, non-small cell lung cancer, stomach cancer or ovarian cancer and is useful in treating human subjects afflicted with bladder cancer, cervical cancer, malignant mesothelioma, non-small cell lung cancer, stomach cancer or ovarian cancer when administered to the subject alone in an amount of from 0.25 mg to 7.5 mg or an amount from 0.1 mg/m² to 5.0 mg/m² or when administered in the aforementioned amounts in combination with cisplatin or doxorubicin.

### Example 6

### Mad2 Overexpressing Cancers

Compound LB-100 inhibits PP2A phosphatase in cancer cells which in turn induces Mad-2 phosphorylation while suppressing Mad-2 protein levels. In order to characterize the effects of LB-100 on cancers overexpressing Mad2, tumor cell lines carrying Mad2-overexpressing mutations are tested. LB-100 inhibits the growth of the cancer cells in in vitro assays. Dose-dependent cytotoxicity is shown. Exposure to LB-100 reduces the expression of Mad-2 in the cells.

Mad2-overexpressing HeLa cells (HeLa Mad-2 O/E), osteosarcoma human cells (OS-17) and gastric cell lines (MKN28, MKN45, MGC803, and SGC7901) express high levels of Mad2 (Yu, L. et al. 2010). LB-100 inhibits the growth of the HeLa cells, OS-17, MKN28, MKN45, MGC803, and SGC7901 cells in vitro. Dose-dependent cytotoxicity is shown in all cell lines, with a half maximal inhibitory concentration. Exposure to LB-100 reduces the expression of Mad-2 in all cell lines.

Gastric cell lines MKN28, MKN45, MGC803, SGC7901 and KATOIII are commercially available from ATCC (Manassas, Va., USA).

Mice are injected subcutaneously with Mad2-overexpressing cancer cells. After an appropriate tumor volume is reached, tumor-bearing mice are randomly allocated to two groups: control group, and LB-100 group. LB-100 is injected intraperitoneally (i.p.). Control mice are injected with DMSO or PBS on the same schedule as the drug treated animals. Tumor size is monitored periodically. All mice are sacrificed at after a predetermined number of day, and xenografts are obtained and weighed. LB-100 significantly reduces tumor burden in the xenografts relative to the control group.

An amount of compound LB-100 is administered to a subject afflicted with a Mad2-overexpressing cancer. The amount of the compound is effective to treat the cancer. The expression levels of Mad2 in cancers cells of a subject afflicted with cancer are determined and compared to a predetermined reference level of Mad2. The expressions levels in the subject are higher than the predetermined reference level of Mad2 and an amount of compound LB-100 is administered to the subject to treat the cancer.

A GLP immunohistochemistry (IHC) is established using commercially available antibodies including, but not limited to, anti-Mad2 antibody, and any of the above cell lines known to overexpress Mad2 including, but not limited to, gastric cell lines MKN28, MKN45, MGC803, and SGC7901 (Wang et al. 2009). The cell line KATOII, which does not overexpress Mad2, is used as a control.

Anti-Mad2 antibody is commercially available from BD Biosciences (San Jose, Calif., USA).

The IHC assay for Mad2 is used to select patients that are responsive to treatment with LB-100 and/or other inhibitors of PP2A based on overexpression of Mad2 in the patient's cancer. Mad2 overexpression is also determined in tissue sections on standard pathology slides before patients are treated with LB-100 and/or other inhibitors of PP2A.

LB-100 inhibits PP2A in human subjects afflicted with Mad2 overexpressing cancer and is useful in treating human subjects afflicted with Mad2 overexpressing cancer when administered to the subject alone in an amount of from 0.25 mg to 7.5 mg or an amount from 0.1 mg/m² to 5.0 mg/m² or when administered in the aforementioned amounts in combination with x-radiation or ionizing radiation.

In some embodiments, the Mad2 overexpressing cancer is the cancer is hepatocellular carcinoma, human osteosarcoma, primary liver cancer, gastric cancer, ovarian cancer, endometrial cancer, colorectal cancer, non-small cell lung cancer, soft-tissue sarcoma, seminoma, breast cancer, lymphoma, fibrosarcoma, neuroblastoma, mucinous ovarian cancer, urothelial bladder cancer, squamous cell carcinoma of the uterine cervix, diffuse large cell lymphoma, lung adenoma, hepatoma, intestinal cancer, fibrosarcoma, prostate cancer, angiomyolipoma, mammary adenocarcinoma or acute myelogenous leukemia.

### Example 7

### Reperfusion Injury

LB-100 reduces reperfusion injury in mammalian tissue that has suffered from an ischemia. The mammalian tissue includes, but is not limited to, cardiac tissue, brain tissue and endothelial tissue. LB-100 reduces tissue damage associated with reperfusion injury in the heart of a subject following a myocardial infarction LB-100 reduces vascular leakage associated with reperfusion injury in a subject suffering from sepsis. LB-100 also improved vascular integrity and reduce tissue damage following acute trauma to tissue. See, for example, WO 2014/005080 A1.

LB-100 inhibits PP2A in human subjects afflicted with reperfusion injury and is useful in treating human subjects afflicted with reperfusion injury after suffering an ischemia when administered to the subject in an amount of from 0.25 mg to 7.5 mg or an amount from 0.1 mg/m² to 5.0 mg/m².

LB-100 inhibits PP2A in human subjects afflicted with reperfusion injury and is useful in treating human subjects afflicted with reperfusion injury after suffering a myocardial infarction when administered to the subject in an amount of from 0.25 mg to 7.5 mg or an amount from 0.1 mg/m² to 5.0 mg/m².

LB-100 inhibits PP2A in human subjects afflicted with reperfusion injury and is useful in treating human subjects afflicted with vascular leakage associated with reperfusion injury when administered to the subject in an amount of from 0.25 mg to 7.5 mg or an amount from 0.1 mg/m² to 5.0 mg/m².

LB-100 inhibits PP2A in human subjects afflicted with reperfusion injury and is useful in treating human subjects afflicted with tissue damage following acute trauma when administered to the subject in an amount of from 0.25 mg to 7.5 mg or an amount from 0.1 mg/m² to 5.0 mg/m².

### Example 8

### Type-2 Diabetes

LB-100 increases insulin sensitivity and/or reduces insulin resistance in human patients diagnosed with Type 2 Diabetes. LB-100 reduces Type 2 Diabetes related vascular injury in the liver, muscle, retina and pancreas and reduces Type 2 Diabetes related vascular injury caused by disruption of the endothelial barrier in human patients diagnosed with Type 2 Diabetes. LB-100 reduces complications associated with or caused by Type 2 Diabetes, including, but not limited to, atherosclerosis low glucose tolerance, dyslipidemia, hyperlipidemia, hypertriglyceridemia, and hypercholesterolemia. See, for example, WO 2014/005084 A1.

LB-100 inhibits PP2A in human subjects afflicted with Type 2 Diabetes and is useful in treating human subjects afflicted with Type 2 Diabetes or reducing complications associated with or caused by Type 2 Diabetes when administered to the subject in an amount of from 0.25 mg to 7.5 mg or an amount from 0.1 mg/m² to 5.0 mg/m².

### Example 9

### Loss of Protein Function Diseases

Human cells isolated from patients with Gaucher's disease type 1 or type 3 were treated with LB-100 and glucocerebrosidase (GBA) levels were quantified and compared. GBA levels were significantly higher than in untreated cells. The half-life of GBA was significantly increased as a result of treating Gaucher's disease type 1 or Gaucher's disease type 3 cells with LB-100. Increasing GBA is a novel approach for the treatment of Gaucher's disease. See, for example, NO 2012/162535 A1.

LB-100 inhibits PP2A in human subjects afflicted with Gaucher's disease and is useful in treating human subjects afflicted with Gaucher's disease when administered to the subject in an amount of from 0.25 mg to 7.5 mg or an amount from 0.1 mg/m² to 5.0 mg/m².

### Example 10

### LB-100 in Combination with Radiation

LB-100 increases the therapeutic effectiveness of radiation with a dose enhancement factor of 1.3-1.4 (and up to 2.0). Thus, when radiation is given with LB-100, the total dose of radiation needed to obtain the same degree of anti-cancer activity is reduced by 30-40 to 100%.

Radiation is given daily to a human subject with cancer for 5 days every week for up to 6 weeks with LB-100 doses of 1.5 to 3.0 mg/m² given every day of radiation or from 1.5 to 4.5 mg/m² on days 1/3/5 of each week of radiation.

Radiation is also given in a single dose 2 hours after a single dose of LB-100 at 4.5 mg/m² or on day 2 of two days of LB-100 also at 4.5 mg/m² each day.

### Example 11

### LB-100 in Combination with Other Chemotherapeutic Agents

LB-100 increases the therapeutic effectiveness of chemotherapeutic agents including, but not limited to vincristine, docetaxel, cisplatin, doxorubicin, and temozolomide. Each of these agents is administered in combination with LB-100. Since LB-100 increases the cytoxicity of these drugs against cancer cells, each agent can be administered at 50-90% of their standard doses in absence of LB-100.

### Discussion

Inhibition of PP2A by the novel inhibitors LB-100 and LB-102 and other structural homologs of these compounds have been shown to result in increased phosphorylation of Akt (Lu et al. 2009: U.S. Pat. No. 8,0858,268). Phosphorylation of Akt leads to its activation, which in turn increases the phosphorylation of several proteins affecting mitochondrial function and mediating cell death (Tsang et al. 2005).

### LB-100 Dosing

Inhibition of PP2A in many cancers potentiates the anticancer activity of a second drug or radiation by interfering with the processes collectively known as the DNA damage and repair response system and with regulation of cell cycle. Because these systems are frequently impaired by acquired mutations in cancer cells but not normal cells, inhibition of PP2A damages the cancer cells and not the normal cells in which all DNA damage repair and cell cycle regulatory systems are functioning. Cancer cells are already “wounded” by mutations and cannot tolerate impairment of other systems dependent on PP2A and are therefore more susceptible to LB-100 than normal cells.

Disclosed herein clinical dosing date showing that administration of LB-100 to cancer patients in the given amounts results in no dose limiting toxicity and is effective in stabilizing the cancer.

LB-100, a novel small molecule inhibitor of PP2A, inhibits the growth of a broad spectrum of leukemic and solid tumor cell lines. In addition, LB100 potentiates the effectiveness of cytotoxic drugs (cisplatin, docetaxel, doxorubicin, temozolomide) and radiation without significant increases in toxicity. The predominant mechanisms responsible for potentiation are inhibition of mitotic exit and homologous recombination repair.

Presented herein is data related to an open label, first-time-in-human, multicenter, phase 1 study of LB-100 in patients with advanced cancer refractory to standard therapies. The first part of the study determines the maximum tolerated dose (MTD) of LB-100 as a single agent when given intravenously days 1-3 every 21 days. Utilizing a standard 3+3 design, patients (pts) are evaluated for dose limiting toxicities (DLT) through 2 cycles. Once the single agent MTD is determined, the dose is reduced by 2 dose levels (DL) and combined with docetaxel given on day 2. Escalation continues until the MTD of the combination is determined. Plasma sampling for pharmacokinetics of LB-100 is collected on days 1 and 3 of cycle 1 in the MTD confirmation cohort.

The starting dose of LB-100 was 0.25 mg/m² and 21 pts have enrolled in part 1 of the study through six dose levels. At DL6, pts received 2.33 mg/m² and no DLTs have been observed. One pt with metastatic colon cancer at DL6 had a grade 2 creatinine after 2 doses that resolved with hydration. This was related to LB-100 and the study was amended to increase the volume and infusion time. One pt on DL3 with stage 4 pancreas cancer had stable disease through 15 cycles of treatment and another pt on DL5 with metastatic thymoma remains on treatment through 8 cycles. Stable disease for 4-6 cycles was also observed in breast, ovarian, carcinoid and testicular cancer patients.

Rb and p53 mutations are common in malignancies leading to chromosomal instability and overexpression of the mitotic checkpoint gene Mad2. PP2A inhibition results in synthetic lethality of cancer cells overexpressing Mad2 which may be a biomarker for LB100 responsiveness. Through 6 DLs, LB-100 has been well tolerated without any DLTs and early activity has been observed with stabilization of disease in a wide variety of cancers.

### Triple Negative Breast Cancer

LB-100 enhances cytotoxicity of DNA damaging and mitosis targeting agents by inhibiting several steps essential to efficient repair of DNA damage, including transient inhibition of the p53 DNA-damage response and impairment of mitotic exit. Of particular relevance to the use of LB-100 in combination with a chemotherapeutic agent for treatment of TNBC (TNBC with or without BRCA1 mutations) is its inhibition of homologous recombination repair (HRR) (Wei et al 2013; Kalev et al 2012). As the induction of double stranded DNA cross-linking by platinum anti-cancer drugs requires HRR to recover, the combination of a drug such as cisplatin plus LB-100 is more effective than cisplatin alone, without increased toxicity to the cytotoxic agent alone.

BRCA1 mutations impose an intrinsic genetic defect upon HRR expected to be maximized by LB-100 pharmacologic inhibition of HRR. As there is no consensus for standard treatment of metastatic TNBC, with platinum compounds emerging as agents of interest, clinical evaluation of a platinum compound plus LB-100 is of considerable interest.

### Mad2 Overexpressing Cancer

A number of reports have demonstrated that alterations in the spindle assembly checkpoint (SAC) pathway, specifically overexpression Mad2, can generate aneuploidy and induce tumor formation (Sotillo R, et al. 2007; Kato et al. 2011). During mitosis or meiosis, the SAC prevents commencement of the anaphase until all chromosomes are properly attached to the spindle. MAD2 (mitotic arrest deficient 2) is an essential spindle checkpoint protein and overexpression of Mad2 results in disruption of the SAC and abnormal chromosome segregation. Tumors that even only experience transient Mad2 overexpression and subsequent chromosome instability recur at elevated rates (Sotillo et al. 2010)

Overexpression of Mad2 has been shown to be necessary for the chromosome instability which results when the Rb and the p53 pathways are inhibited (as is the case in many cancers of all types). Schvartzmann et al. provides evidence that Mad2 overexpression is a critical mediator of chromosome instability (Schvartzmann et al. 2011). As they point out, inhibition of the p53 or Rb pathways lead to upregulation of Mad2 and such events are widespread in human malignancies. They further point out that normalization of Mad2 rescues cells form chromosome instability in model systems.

Overexpression of Mad2 is apparently mitigated by intervention of PP2A (Bian et al. 2014). Compound LB-100 (see U.S. Pat. No. 7,998,957 B2) is a PP2A inhibitor which has anti-cancer activity when used alone (Lu et al. 2009a) and significantly potentiates in vivo, without observable increase in toxicity, the anti-tumor activity of standard cytotoxic anti-cancer drugs including temozolomide (Lu et al. 2009b, Martiniova et al. 2010), doxorubicin (Zhang et al. 2010), and docetaxel. LB-100 was recently approved for Phase I clinical evaluation alone and in combination with docetaxel and is in clinical trial. LB-100 is also significantly less toxic than of cantharidin. Accordingly, LB-100 and analogs thereof have an improved therapeutic profile relative to cantharidin for use in reducing the expression of Mad2 in cancers that overexpress the protein. Reducing expression of Mad2 in cancers that overexpress the protein inhibits the growth of cancer cells and induces cell death.

Analysis of the tumor phenotype, particular in connection with the expression levels of Mad2, ultimately allows for the development of personalized medicine as subjects with tumors that overexpress Mad2 are susceptible to treatment with LB-100 and analogs.

### REFERENCES

Agoston, A. T. et al. (2007) Retinoblastoma pathway dysregulation causes DNA methyltransferase 1 overexpression in cancer via MAD2-mediated inhibition of the anaphase-promoting complex. Am J Pathol 170(5):1585-1593.
Arnold H K, Sears R C. A tumor suppressor role for PP2A-B56alpha through negative regulation of c-Myc and other key oncoproteins. Cancer Metastasis Rev 2008:27:147-58.
Bian, Y. (2014) Synthetic genetic array screen identifies PP2A as a therapeutic target in Mad2-overexpressing tumors. 111(4): 1628-1633.
Chen W, et al. Identification of specific PP2A complexes involved in human cell transformation. Cancer Cell 2004:5:127-36.
Choi, J. M. et al. (2013) High expression of spindle assembly checkpoint proteins CDC20 and MAD2 is associated with poor prognosis in urothelial bladder cancer. Virchows Arch. 463(5):681-7.
Cohen M V and Downey J M (2011) Is it time to translate ischemic preconditioning's mechanism of cardioprotection into clinical practice? J of Cardiovascular Pharmacology and Therapeutics 16(3-4):273-280.
Fung, M. K. et al. (2007) MAD2 expression and its significance in mitotic checkpoint control in testicular germ cell tumour. Biochim Biophys Acta 1773(6):821-832.
Gwinn D, Sweet-Cordero E A. The Phosphatase PP2A Links Glutamine to the Tumor Suppressor p53. Mol Cell 2013; 50:157-8.
Hart M E, et al. Modified norcantharidins; synthesis, protein phosphatases 1 and 2A inhibition, and anticancer activity. Bioorg Med Chem Lett 2004:14:1969-73.
Hisaoka, N. et al. (2000) Aberrant MAD2 expression in soft-tissue sarcoma. Pathol Int 58(6):329-333.
Junttila M R, et al. CIP2A inhibits PP2A in human malignancies. Cell 2007; 130:51-62.
Kalev, P. Loss of PPP2R2A inhibits homologous recombination DNA repair and predicts tumor sensitivity to PARP inhibition. Cancer Res. 2012 December 15; 72(24):6414-24.
Kato, T. et al. (2011) Overexpression of MAD2 predicts clinical outcome in primary lung cancer patients. Lung Cancer 74(1):124-131.
Kim, Y. et al. (2014) MAD2 and CDC20 are Upregulated in High-grade Squamous Intraepithelial Lesions and Squamous Cell Carcinomas of the Uterine Cervix. Int J Gynecol Pathol. 33(5):517-23.
Kim, H. Y. & Parl, J. Molecular targeted therapy for hepatocellular carcinoma: Current and future. Dig Dis 2011; 29; 303-309.
Li W, et al. Cantharidin, a potent and selective PP2A inhibitor, induces an oxidative stress-independent growth inhibition of pancreatic cancer cells through G2/M cell-cycle arrest and apoptosis. Cancer Sci 2010; 101:1226-33.
Li, L. et al. (2013) Combination analysis of Bub1 and Mad2 expression in endometrial cancer: Act as a prognostic factor in endometrial cancer. Arch Gynecol Obstet 288(1):155-165.
Liu D, Chen Z. The effects of cantharidin and cantharidin derivates on tumour cells. Anticancer Agents Med Chem 2009; 9:392-6.
Llovet J M, et al. Sorafenib in advanced hepatocellular carcinoma. N Engl J Med 2008, 359:378-90.
Longacre L S et al (2011) New horizons in cardio protection. Circulation 124:1172-1179.
Lu J, Zhuang Z, Song D K, Mehta G U, Ikejiri B, Muschlen H, Park D M, Lonser R R. (2009a) The effect of a PP2A inhibitor on the nuclear receptor corepressor pathway in glioma. J Neurosurg December 11. 1-9. (DOI: 10.3171/2009.11.JNS091272).
Lu J, Kovach J S, Johnson F, Chiang J, Hodes R, Lonser R, Zhuang Z. (2009b) Inhibition of serine/threonine phosphatase PP2A enhances cancer chemotherapy by blocking DNA damage induced defense mechanisms. PROC Natl Acad Sci 106(28) 11697-11702.
Martiniova, L, Lu J, Chiang J, Bernardo M, Lonser R, Zhuang Z, Pacak K. (2011) Pharmacologic modulation of serine/threonine phosphorylation highly sensitizes PHEO in a MPC cell and mouse model to conventional chemotherapy. PLoS ONE 6(2):e14678.doi:10.1371/journal.pone.0014678.
McCluskey A, et al. Anhydride modified cantharidin analogues: synthesis, inhibition of protein phosphatases 1 and 2A and anticancer activity. Bioorg Med Chem Lett 2000:10:1687-90.
Mehta, R. S. Dose-dense and/or metronomic schedules of specific chemotherapies consolidate the chemosensitivity of triple-negative breast cancer: a step toward reversing triple-negative paradox. J Clin Oneal 26 (19): 3286-8.
Musacchio, A. et al. (May 2007). “The spindle-assembly checkpoint in space and time”. Nat Rev Mol Cell Biol 8 (5): 379-393.
Mumby M. PP2A: unveiling a reluctant tumor suppressor. Cell 2007; 130:21-4.
Nakano, Y. et al. (2012) Mitotic arrest deficiency 2 induces carcinogenesis in mucinous ovarian tumors. Oncol Lett 3(2):281-286.
Reid M A, et al. The B55alpha Subunit of PP2A Drives a p53-Dependent Metabolic Adaptation to Glutamine Deprivation. Mol Cell 2013; 50:200-11.
Schartzman, J. M. et al. (2010) Mitotic chromosomal instability and cancer: Mouse modelling of the human disease. Nat Rev Cancer 10(2):102-115.
Schartzman, J. M. et al. (2011) Mad2 is a critical mediator of the chromosome instability observed upon Rb and p53 pathway inhibition. Cancer Cell 19(6):701-714.
Silver, D. P. et al. Efficacy of Neoadjuvant Cisplatin in Triple-Negative Breast Cancer. JOURNAL OF CLINICAL ONCOLOGY 2010; 28; 7; 1145-1153.
Sotillo, R. et al. (2007) Mad2 overexpression promotes aneuploidy and tumorigenesis in mice. Cancer Cell 11(1):9-23.
Suganuma N, et al. Okadaic acid: an additional non-phorbol-12-tetradecanoate-13-acetate-type tumor promoter. Proc Natl Acad Sci USA 1988; 85:1768-71.
Tanaka, K. et al. (2001) Mitotic checkpoint protein hsMAD2 as a marker predicting liver metastasis of human gastric cancers. Jpn J Cancer Res 92(9):952-958.
Tsang A et al. (2005) Myocardial postconditioning: reperfusion injury revisited. American Journal of Physiology Heart Circulation Physiology 289: H2-H7.
van Deursen, J. M. (2007) Rb loss causes cancer by driving mitosis mad. Cancer Cell 11(1): 1-3.
Van Hoof C, Goris J. PP2A fulfills its promises as tumor suppressor: which subunits are important? Cancer Cell 2004:5:105-6.
Wang, L. et al. (2009) MAD2 as a key component of mitotic checkpoint: A probable prognostic factor for gastric cancer. Am J Clin Pathol 131(6):793-801.
Wassmann, K. et al. (2003) Mad2 phosphorylation regulates its association with Mad1 and the APC/C. EMBO J 22(4):797-806.
Wei D, et al. Inhibition of protein phosphatase 2A radiosensitizes pancreatic cancers by modulating CDC25C/CDK1 and homologous recombination repair. Clin Cancer Res, 2013:19:4422-32.
Wei W, et al. Hypoxia induces a phase transition within a kinase signaling network in cancer cells. Proc Natl Acad Sci USA, 2013; 110:E1352-60.
Wei, D. et al. Inhibition of protein phosphatase 2A radiosensitizes pancreatic cancers by modulating CDC25C/CDK1 and homologous recombination repair. Clin Cancer Res 2013; 19(16):4422-32.
Westermarck J, Hahn W C. Multiple pathways regulated by the tumor suppressor PP2A in transformation. Trends Mol Med 2008:14:152-60.
Yellon D M and Hausenloy D J (2005) Realizing the clinical potential of ischemic preconditioning and postconditioning. Nature Clinical Practice Cardiovascular Medicine 2(11)568-575.
Yi, C. et al. (2000) [Determination of malignant disease-associated DNA-binding protein 2 in patients with liver cancer]. Hua Xi Yi Ke Da Xue Xue Bao 31(3):310-311.
Yu, L. et al. (2010) Mitotic arrest defective protein 2 expression abnormality and its clinicopathologic significance in human osteosarcoma. APMIS 118(3): 222-229.
Zhang, S H. et al. (2008) Clinicopathologic significance of mitotic arrest defective protein 2 overexpression in hepatocellular carcinoma. Hum Pathol 39(12):1827-1834.
Zhuang Z, Lu J, Lonser R, Kovach J S. (2009) Enhancement of cancer chemotherapy by simultaneously altering cell cycle progression and DNA-damage defenses through global modification of the serine/threonine phosphor-proteoame. Cell Cycle 8 (20) 3303-3306.
Zhang C, Peng Y, Wang F, Tan X, Liu N et al. (2010) Enhanced chemosensitivity of sarcoma cells derived from transformed dermal multipotent stem cells by pharmacologic modulation of serine/threonine phosphatase 2A. Biomaterials 31: 9535-9543.
U.S. Patent Application Publication 2009/0018142 A9, published Jan. 15, 2009.
U.S. Patent Application Publication 2009/0035292 A1, published Feb. 5, 2009.
U.S. Patent Application Publication 2010/0029683 A1, published Feb. 4, 2010.
U.S. Patent Application Publication 2010/0029640 A1, published Feb. 4, 2010.
U.S. Pat. No. 7,998,957 B2, issued Aug. 16, 2011.
U.S. Pat. No. 8,227,473 issued Jul. 24, 2012.
PCT International Application Publication No. WO 2014/005080 A1, published Jan. 3, 2014.
PCT International Application Publication No. WO 2014/005084 A1, published Jan. 3, 2014.
PCT International Application Publication No. WO 2012/162535 A1, published Nov. 29, 2012

1. A method of inhibiting protein phosphatase 2A (PP2A) in a human subject in need thereof comprising administering to the subject an amount of from 0.1 mg/m² to 5 mg/m² of a compound having the structure 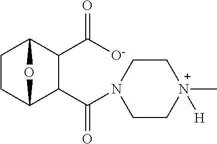 [SMILES: C[NH+]1CCN(C(=O)C2C(C(=O)[O-])[C@H]3CC[C@@H]2O3)CC1]
or a salt, zwitterion, or ester thereof, so as to thereby inhibit protein phosphatase 2A (PP2A) in the subject.

2. The method of claim 1, therein the subject in need thereof is afflicted with a disease or condition mediated by normal expression, overexpression, or under expression of protein phosphatase 2A (PP2A).

3. The method of claim 2, wherein the amount of the compound treats the disease or condition mediated by the normal expression, overexpression, or under expression of protein phosphatase 2A (PP2A) or the inhibition of protein phosphatase 2A (PP2A) in the subject treats the disease or condition mediated by the normal expression, overexpression, or under expression of protein phosphatase 2A (PP2A).

4. (canceled)

5. The method of claim 3, wherein the disease or condition mediated by the normal expression, overexpression, or under expression of protein phosphatase 2A (PP2A) is cancer, a reperfusion injury, a disease characterised by loss of protein function or type-2 diabetes.

6.-8. (canceled)

9. The method of claim 1, wherein the amount of the compound administered is 0.25 mg/m² to 2.5 mg/m², 2.5 mg/m² to 5 mg/m², or 3 mg/m² to 4.5 mg/m².

10.-12. (canceled)

13. The method of claim 9, wherein the amount of the compound administered is about 0.25 mg/m², 0.5 mg/m², 0.75 mg/m², 1.0 mg/m², 1.25 mg/m², 1.5 mg/m², 1.75 mg/m², 2.0 mg/m², 2.25 mg/m², 2.5 mg/m² or 2.75 mg/m².

14. (canceled)

15. The method of claim 1, wherein the amount of the compound is administered once daily.

16.-19. (canceled)

20. The method of claim 1, wherein the amount of the compound is administered on three separate days during week 1 of a twenty-one day treatment cycle.

21.-29. (canceled)

30. The method of claim 5, wherein the subject is afflicted with a cancer.

31. The method of claim 30, wherein the cancer is triple negative breast cancer, bladder cancer, cervical cancer, malignant mesothelioma, non-small call lung cancer, stomach cancer, ovarian cancer, hepatocellular carcinoma, human osteosarcoma, primary liver cancer, gastric cancer, ovarian cancer, endometrial cancer, colorectal cancer, soft-tissue sarcoma, seminoma, lymphoma, fibrosarcoma, mucinous ovarian cancer, urothelial bladder cancer, squamous call carcinoma of the uterine cervix, diffuse large cell lymphoma, lung adenoma, hepatoma, intestinal cancer, prostate cancer, angiomyolipoma, mammary adenocarcinoma, acute myelogenous leukemia, colon cancer, large cell long cancer, adenocarcinoma of the lung, small, cell lung cancer, ovary adenocarcinoma, pancreas carcinoma, prostate carcinoma, promyelocytic leukemia, chronic myelocytic leukemia, acute lymphocytic leukemia, colorectal cancer, non-Hodgkin's lymphoma, Hodgkin's lymphoma, adrenocortical cancer, osteosarcoma, esophageal, gallbladder, head and neck cancer, renal cancer, melanoma, pancreatic cancer, rectal cancer, thyroid cancer, threat cancer, glioblastoma multiforme, neuroblastoma, medulloblastoma, testicular cancer, triple negative breast cancer, chronic myelocytic leukemia (CML), is meningioma, malignant (anaplastic) meningioma, atypical teratoid rhabdoid tumor (ATRT), malignant rhabdoid tumor or diffuse intrinsic pontine glioma (DIPD), carcinoid, or testicular cancer.

32.-40. (canceled)

41. The method of claim 31, wherein cells of the cancer overexpress Mad2.

42. The method of claim 41, wherein the cancer is pancreatic cancer and the cells of the pancreatic cancer overexpress Mad2.

43. The method of claim 30, further comprising the administration of a chemotherapeutic agent to the human subject.

44.-46. (canceled)

47. The method of claim 43, wherein the chemotherapeutic agent is x-radiation, ionising radiation, a DNA damaging agent, a DNA intercalating agent, a microtubule stabilising agent, a microtubule destabilizing agent, a spindle toxin, a platinum-based agent, an anthracycline agent, abarelix, aldesleukin, alemtuzumab, alitertinoin, allopurinol, altretamine, amifostin, anakinra, anastrozole, arsenic trioxide, asparaginase, azacitidine, bevacizumab, bexarotene, bleomycin, bortezomib, busulfan, calusterone, capecitabine, carboplatin, carmustine, celecoxib, cetuximab, chlorambucil, cisplatin, cladribine, clofarabine, cyclophosphamide, cytarabine, decarbazine, dactinomycin, actinomycin D, dalteparin sodium, darbepoetin alfa, dasatinib, daunorubicin, daunomycin, decitabine, denileukin, dexrazoxane, docetaxel, doxorubicin, dromostanolone propionate, exulizumab, epirubicin, epoetin alfa, erlotinib, estramustine, etoposide phosphate, etoposide, VP-16, exemestane, fentanyl citrate, filgrastim, floxuridine, fludarabine, fluorouracil, fulvestrant, gefitinib, gemcitabine, gosereline acetate, histrelin acetate, hydroxyurea, ibritumomab tiuxetan, idarubicin, ifosfamide, imatinib mesylate, interferon alfa 2a, interferon alfa 2b, irinotecan, lapatinib ditosylate, lenalidomide, letrozole, leucovrin, leuprolide acetate, levamisole, lomustine, meclorethamine, megestrol acetate, melphalan, mercaptopurine, mesna, methotrexate, methoxsalen, mitomycin C, mitotane, mitoxantrone, nandrolone phenpropionate, nedaplatin, nelarabine, nofetumomab, nitrosoureas, oprelvekin, oxaliplatin, paclitaxel, palifermin, pamidronate, panitumumab, pegademase, pegaspargase, pegfilgrastim, peginterferon alfa 2b, pemetrexed disodium, pentostatin, picoplatin, pipobroman, plicamycin, mithramycin, porfimer sodium, procarbazine, quinacrine, rasburicase, rituximab, sargrmostim, satraplatin, sorafenib, streptozocin sunitinib, sunitinib maleate, talc, tamoxifen, temozolomide, teniposide, VM-26, testolactone, thalidomide, thioguanine, G-TG, thiotepa, topotecan, toremifene, tositumomab, trastuzumab, tretinoin ATPA, triplatin, uracil mustard, valrubicin, vinblastine, vincristine, vinorelbine, vorinostat, zoledronate, or zoledronic acid.

48.-62. (canceled)

63. The method of claim 47, wherein the amount of the compound and the amount of the chemotherapeutic agent when taken together is more effective to treat the subject then when the chemotherapeutic agent is administered alone.

64.-79. (canceled)

80. The method of claim 1, wherein the compound is administered by intravenous infusion.

81.-82. (canceled)

83. The method of claim 5 wherein the subject is afflicted with a reperfusion injury, a disease characterized by a loss of protein function caused by a genetic abnormality associated with the disease or Type 2 Diabetes.

84. The method of claim 83, wherein the treating of the reperfusion injury comprises reducing reperfusion injury in tissue in the subject and the treating of the Type 2 Diabetes comprises increasing the insulin sensitivity of cells in the subject or reducing complications associated with Type 2 Diabetes in the subject.

85.-91. (canceled)

92. The method of claim 83, wherein the disease characterised by a ices of protein function is Gaucher's disease, von Hippel-Lindau disease, cystic fibrosis, Phenylketonuria, Fabry disease, Tay-Sachs disease, Pompe disease, Helmann-Pick disease (Type A, B and C), Marfan syndrome, Hemophilia A & B, retinitis pigmentosa, Neurofibromatosis Type 2, pheochromocytoma, paraganglioma, Multiple Endocrine Neoplasia Type 1, Familial Hypercholesterolemia, Hurler's disease, Hunter syndrome, Sanfilippo syndrome, Morquio syndroms, Maroteaux-Lamy syndrome, Sly syndrome, Sandhoff's disease, Fucosidosis, alpha-mannosidosis, beta-mannosidosis, aspartylglucosaminuria, Sialidosis, Inclusion-cell (I-cell) disease, Pseudo-Hurler polydystrophy, Krabbe's disease, Metachromatic leukodystrophy, multiple sulfatase deficiency, Wolmen's disease, Cholesteryl ester storage disease, Late onset GAA deficiency, Danon's disease, Neutropenia, X-linked hyper IgM syndrome, X-linked agammaglobulinemia, X-linked lymphoproliferative disease, Severe Combined Immunodeficiency, Noonan syndrome, juvenile myelomonocytic leukemia, Basel cell carcinoma, STAT1 deficiency, Alzheimer's disease, Parkinson's disease, Huntington's disease, TTR Amyloid Polyneuropathy, Ataxia Telangiectasia, Creutzfeldt-Jakob disease, Type II diabetes, Hereditary Transthyretin (TTR) amyloidosis, pheochromocytomas (PHEO) or paragangliomas (PGL).

93.-98. (canceled)

99. The method of claim 3, wherein the disease or condition mediated by the normal expression, overexpression, or under expression of protein phosphatase 2a (PP2A) is a neurodegenerative disease mediated by under expression of protein phosphatase 2A (PP2A).

100. The method of claim 99, wherein the neurodegenerative disease is diabetic neuropathy, senile dementias, Alzheimer's disease, Mild Cognitive Impairment (MCI), dementia, Lewy Body Dementia, Frontal Temporal Lobe dementia (Pick's disease), Parkinson's Disease, facial nerve (Bell's) palsy, glaucoma, Huntington's chores, amyotrophic lateral sclerosis (ALS), status epilepticus, non-arteritic optic neuropathy, intervertebral disc herniation, vitamin deficiency, Creutzfeldt-Jakob disease, carpal tunnel syndrome, peripheral neuropathies, uremia, porphyria, hypoglycemia, Sjorgren Larsson syndrome, acute sensory neuropathy, chronic ataxic neuropathy, biliary cirrhosis, primary amyloidosis, obstructive lung diseases, acromegaly, malabsorption syndromes, polycythemia vera, IgA and IgG gammapathies, Charcot-Marie-Tooth disease, ataxia telangectasia, Friedreich's ataxia, amyloid polyneuropathies, adrenomyeloneuropathy, Giant axonal neuropathy, Refsum's disease, Fabry's disease and lipoproteinemia, Progressive Supranuclear Palsy or Corticobasal degeneration.
